SEQUENCE LISTING (60 sequences)

SEQ ID NO: 1 - protein, 17 aa, synthetic construct
  REGION 1..17: note = Artificial Sequence
CAVRLAGYGGSQGNLIF

SEQ ID NO: 2 - protein, 13 aa, synthetic construct
  REGION 1..13: note = Artificial Sequence
CASSSQGTDTQYF

SEQ ID NO: 3 - protein, 6 aa, synthetic construct
  REGION 1..6: note = Artificial Sequence
DSAIYN

SEQ ID NO: 4 - protein, 8 aa, synthetic construct
  REGION 1..8: note = Artificial Sequence
IQSSQREQ

SEQ ID NO: 5 - protein, 5 aa, synthetic construct
  REGION 1..5: note = Artificial Sequence
SGHDY

SEQ ID NO: 6 - protein, 6 aa, synthetic construct
  REGION 1..6: note = Artificial Sequence
FNNNVP

SEQ ID NO: 7 - protein, 276 aa, synthetic construct
  REGION 1..276: note = Artificial Sequence
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRLAGYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 8 - protein, 311 aa, synthetic construct
  REGION 1..311: note = Artificial Sequence
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSQGTDTQYFGPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 9 - protein, 237 aa, synthetic construct
  REGION 1..237: note = Artificial Sequence
KQEVTQIPAALSVPEGENLVLNCSFTDSAIYNLQWFRQDPGKGLTSLLLIQSSQREQTSGRLNASLDKSSGRSTLYIAASQPGDSATYLCAVRLAGYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFR

SEQ ID NO: 10 - protein, 263 aa, synthetic construct
  REGION 1..263: note = Artificial Sequence
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSQGTDTQYFGPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYE

SEQ ID NO: 11 - protein, 237 aa, synthetic construct
  REGION 1..237: note = Artificial Sequence
KQEVTQIPAALSVPEGENLVLNCSFTDSAIYNLQWFRQDPGKGLTSLLLIQSSQREQTSGRLNASLDKSSGRSTLYIAASQPGDSATYLCAVRLAGYGGSQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFR

SEQ ID NO: 12 - protein, 263 aa, synthetic construct
  REGION 1..263: note = Artificial Sequence
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSQGTDTQYFGPGTRLTVLEDLKNVFPPKVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADCGFTSESYQQGVLSATILYE

SEQ ID NO: 13 - protein, 263 aa, synthetic construct
  REGION 1..263: note = Artificial Sequence
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSQGTDTQYFGPGTRLTVLEDLKNVFPPKVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYE

SEQ ID NO: 14 - protein, 116 aa, synthetic construct
  REGION 1..116: note = Artificial Sequence
KQEVTQIPAALSVPEGENLVLNCSFTDSAIYNLQWFRQDPGKGLTSLLLIQSSQREQTSGRLNASLDKSSGRSTLYIAASQPGDSATYLCAVRLAGYGGSQGNLIFGKGTKLSVKP

SEQ ID NO: 15 - protein, 113 aa, synthetic construct
  REGION 1..113: note = Artificial Sequence
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSQGTDTQYFGPGTRLTVL

SEQ ID NO: 16 - DNA, 1839 bp, synthetic construct
  misc_feature 1..1839: note = Artificial Sequence
  source 1..1839: mol_type = other DNA
atgggcagctggaccctgtgttgtgtgtccctgtgtatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttttgtgccagcagcagccagggcaccgacacacagtattttggccctggcaccagactgaccgtgctggaagatctgaagaacgtgttccctccaaaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagaacagcccgctctgaacgacagccggtactgtctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgctctggtgctgatggccatggtcaagcggaaggactctagaggcagagccaagagaagcggctctggcgaaggcagaggtagcctgcttacatgcggcgacgtggaagagaaccccggacctatggaaacactgctgggcctgctgatcctgtggctgcaactgcaatgggtgtccagcaagcaagaagtcacccagattccagccgctctgtctgtgcctgagggcgaaaacctggtcctgaactgcagcttcaccgacagcgccatctacaacctgcagtggttcagacaggaccccggcaagggactgacaagcctgctgctgattcagagcagccagagagagcagaccagcggcagactgaatgccagcctggataagtcctccggcagaagcaccctgtatatcgccgcatctcagcctggcgatagcgccacatatctgtgcgccgttagactggccggctatggcggatctcagggcaatctgatcttcggcaagggcaccaagctgagcgtgaagcccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagacagtgctggacatgcggagcatggacttcaagagcaacagcgccgtggcctggtccaacaagagcgatttcgcctgcgccaacgccttcaacaatagcattatccccgaggacacattcttcccaagtcctgagagcagctgcgacgtgaagctggtggaaaagagcttcgagacagacaccaacctgaacttccagaacctgtccgtgatcggcttccggatcctgctcctgaaagtggccggcttcaacctgctgatgaccctgagactgtggtccagc

SEQ ID NO: 17 - protein, 5 aa, synthetic construct
  REGION 1..5: note = Artificial Sequence
GGGGS

SEQ ID NO: 18 - DNA, 21 bp, synthetic construct
  misc_feature 1..21: note = Artificial Sequence
  source 1..21: mol_type = other DNA
gagaccctcaggcggctgctc

SEQ ID NO: 19 - DNA, 24 bp, synthetic construct
  misc_feature 1..24: note = Artificial Sequence
  source 1..24: mol_type = other DNA
tgtgtggccaggcacaccagtgtg

SEQ ID NO: 20 - protein, 10 aa, synthetic construct
  REGION 1..10: note = Artificial Sequence
EQKLISEEDL

SEQ ID NO: 21 - protein, 8 aa, synthetic construct
  REGION 1..8: note = Artificial Sequence
DYKDDDDK

SEQ ID NO: 22 - protein, 9 aa, synthetic construct
  REGION 1..9: note = Artificial Sequence
YPYDVPDYA

SEQ ID NO: 23 - protein, 10 aa, synthetic construct
  REGION 1..10: note = Artificial Sequence
ELAGIGILTV

SEQ ID NO: 24 - protein, 19 aa, synthetic construct
  REGION 1..19: note = Artificial Sequence
ATNFSLLKQAGDVEENPGP

SEQ ID NO: 25 - protein, 20 aa, synthetic construct
  REGION 1..20: note = Artificial Sequence
QCTNYALLKLAGDVESNPGP

SEQ ID NO: 26 - protein, 22 aa, synthetic construct
  REGION 1..22: note = Artificial Sequence
VKQTLNFDLLKLAGDVESNPGP

SEQ ID NO: 27 - protein, 18 aa, synthetic construct
  REGION 1..18: note = Artificial Sequence
EGRGSLLTCGDVEENPGP

SEQ ID NO: 28 - protein, 4 aa, synthetic construct
  REGION 1..4: note = Artificial Sequence
SGSG

SEQ ID NO: 29 - protein, 257 aa, synthetic construct
  REGION 1..257: note = Artificial Sequence
KQEVTQIPAALSVPEGENLVLNCSFTDSAIYNLQWFRQDPGKGLTSLLLIQSSQREQTSGRLNASLDKSSGRSTLYIAASQPGDSATYLCAVRLAGYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 613 aa, synthetic construct
  REGION 1..613: note = Artificial Sequence
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSQGTDTQYFGPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRLAGYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 31 - protein, 292 aa, synthetic construct
  REGION 1..292: note = Artificial Sequence
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSQGTDTQYFGPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 32 - protein, 10 aa, synthetic construct
  REGION 1..10: note = Artificial Sequence
YGGSQGNLIF

SEQ ID NO: 33 - protein, 17 aa, synthetic construct
  REGION 1..17: note = Artificial Sequence
CALSSYHYGGSQGNLIF

SEQ ID NO: 34 - protein, 17 aa, synthetic construct
  REGION 1..17: note = Artificial Sequence
CAVREADYGGSQGNLIF

SEQ ID NO: 35 - protein, 17 aa, synthetic construct
  REGION 1..17: note = Artificial Sequence
CAVRFGYYGGSQGNLIF

SEQ ID NO: 36 - protein, 12 aa, synthetic construct
  REGION 1..12: note = Artificial Sequence
CASRGNTGELFF

SEQ ID NO: 37 - protein, 14 aa, synthetic construct
  REGION 1..14: note = Artificial Sequence
CASRTGQGNQPQHF

SEQ ID NO: 38 - protein, 11 aa, synthetic construct
  REGION 1..11: note = Artificial Sequence
CASSLEQGQYF

SEQ ID NO: 39 - protein, 6 aa, synthetic construct
ATGYPS

SEQ ID NO: 40 - protein, 7 aa, synthetic construct
ATKADDK

SEQ ID NO: 41 - protein, 5 aa, synthetic construct
VGISA

SEQ ID NO: 42 - protein, 5 aa, synthetic construct
LSSGK

SEQ ID NO: 43 - protein, 5 aa, synthetic construct
MDHEN

SEQ ID NO: 44 - protein, 6 aa, synthetic construct
SYDVKM

SEQ ID NO: 45 - protein, 115 aa, synthetic construct
DSVTQMEGPVTLSEEAFLTINCTYTATGYPSLFWYVQYPGEGLQLLLKATKADDKGSNKGFEATYRKETTSFHLEKGSVQVSDSAVYFCALSSYHYGGSQGNLIFGKGTKLSVKP

SEQ ID NO: 46 - protein, 115 aa, synthetic construct
GVIQSPRHEVTEMGQEVTLRCKPISGHDYLFWYRQTMMRGLELLIYFNNNVPIDDSGMPEDRFSAKMPNASFSTLKIQPSEPRDSAVYFCASSLCASRGNTGELFFGEGSRLTVL

SEQ ID NO: 47 - protein, 115 aa, synthetic construct; identical to SEQ ID NO: 45

SEQ ID NO: 48 - protein, 117 aa, synthetic construct
GVIQSPRHEVTEMGQEVTLRCKPISGHDYLFWYRQTMMRGLELLIYFNNNVPIDDSGMPEDRFSAKMPNASFSTLKIQPSEPRDSAVYFCASSLCASRTGQGNQPQHFGDGTRLSIL

SEQ ID NO: 49 - protein, 112 aa, synthetic construct
NEVEQSPQNLTAQEGEFITINCSYSVGISALHWLQQHPGGGIVSLFMLSSGKKKHGRLIATINIQEKHSSLHITASHPRDSAVYICAVRLAGYGGSQGNLIFGKGTKLSVKP

SEQ ID NO: 50 - protein, 108 aa, synthetic construct
SRYLVKRTGEKVFLECVQDMDHENMFWYRQDPGLGLRLIYFSYDVKMKEKGDIPEGYSVSREKKERFSLILESASTNQTSMYLCASSLCASSLEQGQYFGPGTRLLVL

SEQ ID NO: 51 - protein, 115 aa, synthetic construct; identical to SEQ ID NO: 45

SEQ ID NO: 52 - protein, 103 aa, synthetic construct
SRYLVKRTGEKVFLECVQDMDHENMFWYRQDPGLGLRLIYFSYDVKMKEKGDIPEGYSVSREKKERFSLILESASTNQTSMYLCASSLEQGQYFGPGTRLLVL

SEQ ID NO: 53 - protein, 236 aa, synthetic construct
DSVTQMEGPVTLSEEAFLTINCTYTATGYPSLFWYVQYPGEGLQLLLKATKADDKGSNKGFEATYRKETTSFHLEKGSVQVSDSAVYFCALSSYHYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFR

SEQ ID NO: 54 - protein, 265 aa, synthetic construct
GVIQSPRHEVTEMGQEVTLRCKPISGHDYLFWYRQTMMRGLELLIYFNNNVPIDDSGMPEDRFSAKMPNASFSTLKIQPSEPRDSAVYFCASSLCASRGNTGELFFGEGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYE

SEQ ID NO: 55 - protein, 236 aa, synthetic construct; identical to SEQ ID NO: 53

SEQ ID NO: 56 - protein, 267 aa, synthetic construct
GVIQSPRHEVTEMGQEVTLRCKPISGHDYLFWYRQTMMRGLELLIYFNNNVPIDDSGMPEDRFSAKMPNASFSTLKIQPSEPRDSAVYFCASSLCASRTGQGNQPQHFGDGTRLSILEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYE

SEQ ID NO: 57 - protein, 233 aa, synthetic construct
NEVEQSPQNLTAQEGEFITINCSYSVGISALHWLQQHPGGGIVSLFMLSSGKKKHGRLIATINIQEKHSSLHITASHPRDSAVYICAVRLAGYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFR

SEQ ID NO: 58 - protein, 258 aa, synthetic construct
SRYLVKRTGEKVFLECVQDMDHENMFWYRQDPGLGLRLIYFSYDVKMKEKGDIPEGYSVSREKKERFSLILESASTNQTSMYLCASSLCASSLEQGQYFGPGTRLLVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYE

SEQ ID NO: 59 - protein, 236 aa, synthetic construct; identical to SEQ ID NO: 53

SEQ ID NO: 60 - protein, 253 aa, synthetic construct
SRYLVKRTGEKVFLECVQDMDHENMFWYRQDPGLGLRLIYFSYDVKMKEKGDIPEGYSVSREKKERFSLILESASTNQTSMYLCASSLEQGQYFGPGTRLLVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYE

(19) United States
(12) Patent Application Publication
(10) Pub. No.: US 2025/0064854 A1
(43) Pub. Date: Feb. 27, 2025

(54) NOVEL T-CELL RECEPTOR

(71) Applicant: University College Cardiff Consultants, Cardiff (GB)

(72) Inventors: Andrew Sewell, Cardiff (GB); Garry Dolton, Cardiff (GB); Michael Douglas Crowther, Cardiff (GB)

(21) Appl. No.: 18/793,200

(22) Filed: Aug. 2, 2024

Related U.S. Application Data
(63) Continuation of application No. PCT/GB2023/050236, filed on Feb. 3, 2023.

(30) Foreign Application Priority Data
Feb. 3, 2022 (EP) 22155065.0

(51) Int. Cl. A61K 35/17; A61K 39/00; C07K 14/725; C12N 5/0783; C12N 15/86

(52) U.S. Cl. CPC A61K 35/17; A61K 39/4611; A61K 39/4632; A61K 39/4644; C07K 14/7051; C12N 5/0636; C12N 15/86; C07K 2317/565; C12N 2510/00; C12N 2740/15043

(57) ABSTRACT

The present disclosure relates inter alia to a new T-cell receptor (TCR), an immune cell, particularly a T-cell expressing said TCR; a vector encoding said TCR; a soluble version of said TCR; a pharmaceutical composition or bispecific comprising said TCR, and a method of treating cancer using said TCR, said cell, said vector, said pharmaceutical composition or bispecific comprising said TCR.

### FIELD OF THE INVENTION

The present disclosure relates inter alia to a new T-cell receptor (TCR), an immune cell, particularly a T-cell expressing said TCR; a vector encoding said TCR; a soluble version of said TCR; a pharmaceutical composition or bispecific comprising said TCR, and a method of treating cancer using said TCR, said cell, said vector, said pharmaceutical composition or bispecific comprising said TCR.

### BACKGROUND

Our earlier disclosure WO2019/081902 describes a new type of T-cell and corresponding receptor effective for treating cancer, which recognizes cancer cells through the population-invariant major histocompatibility complex class I-related protein 1 (MR1) (also detailed in Crowther et al. Nature Immunology, 2020). The identification of this new type of T-cell stemmed from experiments searching for T-cells recognising cancer cells without the requirement for a specific Human Leukocyte Antigen (HLA). The HLA locus is highly variable with over 17,000 different alleles having been described today. As such, any therapeutic approach that works via an HLA can only be effective in a subset of patients. In contrast, the entire human population is thought to express the same MR1.

The main type of MR1-restricted T-cells that are known are called mucosal-associated invariant T-cells (MAITs). MAITs are known to recognise intermediates of mycobacterial riboflavin biosynthesis. Recent studies by our own and other laboratories have shown that there are also other types of MR1-restricted T-cells that recognise different MR1-bound ligands. The work described herein relates to these new type of T-cells that target specificity via MR1. The TCR does not bind to MR1 per se or to MR1 loaded with known infectious disease agent ligands, rather this T-cell recognises a cancer-specific ligand within the MR1 binding groove. Hence, MR1 presents a cancer-specific, or cancer-upregulated, ligand to the TCR.

We have now discovered a new family of T-cell clones which also recognizes cancer cells through MR1 and which comprise a variable region of the α chain which is encoded by a sequence resulting from the recombination of a human T Cell Receptor Alpha Variable (TRAV) gene and human T cell Receptor Alpha Joining Gene TRAJ42, exampled sequences disclosed herein include TRAV21, TRAV41, TRAV 9-2. The foregoing recombination results in the formation of an alpha chain complementarity-determining region 3 (CDR3α) which comprises the amino acid sequence YGGSQGNLIF (SEQ ID NO: 32). Our new T-cell clones, MC.27.759S, A3, A4, C1 and C2 were discovered by priming HLA-A2 negative PBMCs with C1R.A2 cells transduced with MR1 (C1R.A2.MR1). The experimental approach involved incubating the PBMCs with C1R.A2.MR1 and then isolating and cloning MR1-reactive T-cells that had proliferated in response to the C1R.A2.MR1. Further investigations showed that each of clones the MC.27.759S, A3, A4, C1 and C2 were able to recognise and kill cancers cells, including cancer cells from a number of organs and tissue types, thus showing the clone had potential for treating many types of cancer.

The type of TCR to which this invention relates is a disulfide-linked membrane-anchored heterodimeric protein consisting of the highly variable alpha (α) and beta (β) chains that associate with the invariant CD3 chain molecules to form a complete functioning TCR. T-cells expressing this receptor are referred to as α:β (or αβ) T-cells.

The α and β chains are composed of extracellular domains comprising a Constant (C) region and a Variable (V) region. The Constant region is proximal to the cell membrane, followed by a transmembrane region and a short cytoplasmic tail, while the Variable region binds to the ligand. The ligand for most αβ T-cells is a peptide bound to an HLA molecule.

The variable domain is formed of variable regions of both the TCR α-chain and β-chain each of which has three hypervariable regions called complementarity determining regions (CDRs). There is also an additional area of variability on the β-chain (HV4) that does not normally contact antigen and, therefore, is not considered a CDR. In general, the antigen-binding site is formed by the CDR loops of the TCR α-chain and β-chain. CDR1α and CDR2α are encoded by the individual Vα genes whereas CDR1β and CDR2β are encoded by the individual VB genes. The CDR3 of the TCR α-chain is especially hypervariable due to the potential for nucleotide addition and removal around the joining of the variable (V) region and a joining (J) region. The TCR β-chain CDR3 has even more capacity for variation as it can also include a diversity (D) gene.

CDR3 is the main CDR responsible for recognizing HLA-bound antigenic peptides (the usual target for αβ TCRs). However, in some cases CDR1 of the α chain has also been shown to interact with the N-terminal part of the HLA-bound antigenic peptide, and CDR1 of the β-chain may interact with the C-terminal part of the HLA-bound antigenic peptide.

In 2015 about 90.5 million people had cancer. About 14.1 million new cases occur a year (not including skin cancer other than melanoma). It causes about 8.8 million deaths (15.7%) of human deaths. The most common types of cancer in males are lung cancer, prostate cancer, colorectal cancer and stomach cancer. In females, the most common types of cancer are breast cancer, colorectal cancer, lung cancer and cervical cancer. If skin cancer, other than melanoma, were included in total new cancers each year it would account for around 40% of cases. In children, acute lymphoblastic leukaemia and brain tumours are most common except in Africa where non-Hodgkin lymphoma occurs more often. In 2012, about 165,000 children under 15 years of age were diagnosed with cancer. The risk of cancer increases significantly with age and many cancers occur more commonly in developed countries. Rates are increasing as more people live to an old age and as lifestyle changes occur in the developing world. The financial costs of cancer were estimated at $1.16 trillion USD per year as of 2010. It follows that there is a need to provide better and safer ways of treating or eradicating this disease. An immunotherapy that uses the body's natural defence systems to kill aberrant tissue is acknowledged to be safer than chemical intervention but, to be effective, the immunotherapy must be cancer specific. Moreover, the discovery of an immunotherapy that is effective against many types of cancer would be extremely beneficial as not only could it be administered to individuals suffering from many different types of cancer (i.e. it would have pan-population application) but it could also be administered to a single individual suffering from more than one type of cancer. Additionally, the identification of an immunotherapy that was not HLA-restricted would also be extremely advantageous as it means it could be administered to any individual regardless of HLA tissue type.

### SUMMARY OF THE INVENTION

The T-cells of the clones identified by the Inventors herein have the aforementioned advantageous characteristics in that they are effective against many types of cancer and they are not HLA restricted. Thus, the T-cells and receptors of the present invention have pan-population application due to the ubiquitous expression of the MR1 molecule.

According to one aspect of the present invention, there is provided a cancer-specific T-cell receptor (TCR) or a cancer-specific binding fragment of a TCR which binds a tumour antigen, wherein the TCR or binding fragment comprises a variable region of an α chain which is encoded by a sequence resulting from the recombination of a human T Cell Receptor Alpha Variable (TRAV) gene and human T cell Receptor Alpha Joining Gene TRAJ42, optionally the TRAV gene may be any one of TRAV21, TRAV41, TRAV 9-2.

According to another aspect of the present invention, there is provided a cancer-specific T-cell receptor (TCR) or a cancer-specific binding fragment of a TCR which binds a tumour antigen, wherein the TCR or binding fragment comprises an alpha chain complementarity-determining region 3 (CDR3α) which comprises the amino acid sequence YGGSQGNLIF (SEQ ID NO: 32).

According to another aspect of the present invention, there is provided a cancer-specific T-cell receptor (TCR) or a cancer-specific binding fragment of a TCR which binds a tumour antigen, wherein the TCR or binding fragment comprises:
(a) an alpha chain comprising a CDR3α comprising or consisting of CAVRLAGYGGSQGNLIF, (SEQ ID NO: 1) or a variant CDR3α that has 1, 2, 3, 4 or 5 amino acid variations with respect thereto; and/or a beta chain comprising a CDR3β comprising or consisting of CASSSQGTDTQYF, (SEQ ID NO:2) or a variant CDR3β that has 1, 2, 3, 4 or 5 amino acid variations with respect thereto,
(b) an alpha chain comprising a CDR3α comprising or consisting of CALSSYHYGGSQGNLIF (SEQ ID NO: 33) or a variant CDR3α that has 1, 2, 3, 4 or 5 variations amino acid variations with respect thereto; and/or a beta chain comprising a CDR3β comprising or consisting of CASRGNTGELFF (SEQ ID NO: 36) or a variant CDR3β that has 1, 2, 3, 4 or 5 amino acid variations with respect thereto, or
(c) an alpha chain comprising a CDR3α comprising or consisting of CALSSYHYGGSQGNLIF (SEQ ID NO: 33) or a variant CDR3α that has 1, 2, 3, 4 or 5 variations with respect thereto; and/or a beta chain comprising a CDR3β comprising or consisting of CASRTGQGNQPQHF (SEQ ID NO: 37) or a variant CDR3β that has 1, 2, 3, 4 or 5 amino acid variations with respect thereto,
(d) an alpha chain comprising a CDR3α comprising or consisting of CAVREADYGGSQGNLIF (SEQ ID NO: 34) or a variant CDR3α that has 1, 2, 3, 4 or 5 variations with respect thereto; and/or a beta chain comprising a CDR3β comprising or consisting of CASSLEQGQYF (SEQ ID NO: 38) or a variant CDR3β that has 1, 2, 3, 4 or 5 amino acid variations with respect thereto, or
(e) an alpha chain comprising a CDR3α comprising or consisting of CALSSYHYGGSQGNLIF (SEQ ID NO: 33) or a variant CDR3α that has 1, 2, 3, 4 or 5 amino acid variations with respect thereto, such as 1 or 2 variations, such as 1 variation; and/or a beta chain comprising a CDR3β comprising or consisting of CASSLEQGQYF (SEQ ID NO: 38) or a variant CDR3β that has 1, 2, 3, 4 or 5 amino acid variations with respect thereto, wherein in each case the variations are selected from additions, substitutions and deletions.

In another aspect of the present invention, there is provided a polynucleotide encoding the TCR or cancer-specific binding fragment according to the invention and a vector for delivery of a polynucleotide to cells comprising a polynucleotide according to the invention.

In yet another aspect of the present invention, there is provided an immune cell or an immune cell clone, particularly a T-cell or a T-cell clone expressing the TCR or cancer-specific binding fragment according to the invention, i.e. a modified immune cell or immune cell clone.

In a further aspect of the present invention, there is provided an ex vivo process comprising (i) obtaining immune cells, particularly T-cells from a patient, (ii) optionally expanding the immune cells (iii) introducing a heterologous polynucleotide according to the invention or a vector according to the invention into the immune cells, particularly T-cells to produce modified immune cells, particularly T-cells which express a cancer-specific T-cell receptor (TCR) or a cancer-specific binding fragment of a TCR according to the invention; and (iii) reintroducing said modified immune cells, particularly T-cells into the patient.

In yet a further aspect of the present invention, there is provided a method of treatment of cancer comprising administering to a patient in need thereof transduced or modified immune cells, particularly T-cells wherein the immune cells are immune cells that have been obtained from said patient and a heterologous polynucleotide according to the invention or a vector according to the invention has been introduced into the immune cells, particularly T-cells, such that the immune cells, particularly T-cells express a cancer-specific T-cell receptor (TCR) or a cancer-specific binding fragment of a TCR according to the invention.

In another aspect of the present invention, there is provided a pharmaceutical composition comprising the TCR or cancer-specific binding fragment according to the invention, the polynucleotide according to the invention, a vector according to the invention, the immune cell or the immune cell clone (such as the T-cell or the T-cell clone) according to the invention and a pharmaceutically acceptable carrier.

In yet another aspect of the present invention, there is provided a bispecific construct comprising the TCR or cancer-specific binding fragment according to the invention and an immune cell activating component or ligand that binds to and activates an immune cell.

In a further aspect of the present invention, there is provided a fusion protein comprising the TCR or cancer-specific binding fragment according to the present invention and a heterologous protein.

In yet a further aspect of the present invention, there is provided a method of treating cancer in a subject comprising administering a therapeutically effective amount of the immune cell or the immune cell clone (such as the T-cell or T-cell clone) according to the invention, pharmaceutical composition according to the invention, bispecific construct according to the invention or fusion protein according to the invention, to the subject.

In another aspect of the present invention, there is provided a pharmaceutical composition comprising:
a) the immune cell or the immune cell clone (such as the T-cell or the T-cell clone) according to the invention, the pharmaceutical composition according to the invention, bispecific construct according to the invention or the fusion protein according to the invention; and
b) an anti-cancer agent.

### BRIEF DESCRIPTION OF THE DRAWINGS

FIG. 1A shows flow cytometry plots for donor T-cells primed with irradiated C1R.A2 and C1R.A2.MR1 cells and gated for CD107a and TNFα (top), TCRαβ and TCRγδ (middle) and TCRαβ and CD8 (bottom); FIG. 1B shows flow cytometry plots for donor T-cells primed two times with C1R.A2 and C1R.A2.MR1 cells, and gated for CD107a and TNFα (top), CD3 and IFNγ (middle) and CD3 and CD8 (bottom) (see Example 1).

FIG. 2A shows flow cytometry plots for the MC.27.759S T-cells stimulated with various MM909.24 cells, and gated for IFNγ and TNFα (top) and CD3 and CD107a (bottom); FIG. 2B shows histograms for expression of CD8 (left hand plot) and CD4 (right hand plot) in MC.27.759S; part C shows TNFα produced by MC.27.759S cells exposed C1R cells transduced with WT MR1 or the K43A mutant version of MR1 (see Example 1).

FIG. 3A shows percentage lysis of a range of cell lines by MC.27.759S; FIG. 3B shows percentage lysis titration of MM909.24 wildtype and MM909.24 MR1−/− cells by MC.27.759S (see Example 3; MM909.24 is a melanoma cell line derived from a melanoma patient). A549 and A549.C9 are lung cancer lines, M11, M15, M24, M24 MR1−/−, M24.MR1, M29, M37 are skin and melanoma cancer lines, MCF-7 is a breast cancer line and U2OS is a bone cancer line.

FIG. 4A shows flow cytometry plots for T cell clones MC.7.G5 and MC.27.759S stimulated with various target cancer cells and gated for TNFα and CD107a, and FIG. 4B shows the percentage of cells positive for TNFα and CD107a in the clones stimulated with various target cancer cells, knockout cells (KO) are control MR1-ve cells (see Example 1). MM24 (melanoma), A549 (lung adenocarcinome), K562 (myelogenous leukemia), THP1 (monocytic leukemia), MCF7 (breast cancer).

FIG. 5 shows flow cytometry plots for the Donor A and C derived T-cells (from which clones A3, A4 and C1 C2 were obtained) stimulated with various MM909.24 cells, and gated for CD8 and TNFα (see Example 11).

FIG. 6A and FIG. 6B show flow cytometry plots for T cell clones MC.7.G5, MC.27.759S, A3, A4 and C1 stimulated with various target cancer cells and gated for CD69, the various target cancer cells include, knockout cells (KO) and controls and are further set out in Table 2 in Example 11.

### BRIEF DESCRIPTION OF THE SEQUENCES

SEQ ID NO: 1 is CDR3 of the α chain of a TCR according to the invention, (MC.27.759S).

SEQ ID NO: 2 is CDR3 of the β chain of a TCR according to the invention, (MC.27.759S).

SEQ ID NO: 3 is CDR1 of the α chain of a TCR according to the invention, (MC.27.759S).

SEQ ID NO: 4 is CDR2 of the α chain of a TCR according to the invention, (MC.27.759S).

SEQ ID NO: 5 is CDR1 of the β chain of a TCR according to the invention, (MC.27.759S).

SEQ ID NO: 6 is CDR2 of the β chain of a TCR according to the invention, (MC.27.759S).

SEQ ID NO: 7 is the sequence of the native α chain of the TCR of clone MC.27.759S according to the invention.

SEQ ID NO: 8 is the sequence of the native β chain of the TCR of clone MC.27.759S according to the invention.

SEQ ID NO: 9 is the sequence of the extracellular region of the native α chain of the TCR of clone MC.27.759S according to the invention.

SEQ ID NO: 10 is the sequence of the extracellular region of the native β chain of the TCR of clone MC.27.759S according to the invention.

SEQ ID NO: 11 is the sequence of the extracellular region of the α chain of a mutant TCR according to the invention.

SEQ ID NO: 12 is the sequence of the extracellular region of the β chain of a mutant TCR according to the invention.

SEQ ID NO: 13 is the sequence of the extracellular region of the β chain of a mutant TCR according to the invention

SEQ ID NO: 14 is the sequence of the native α chain of the TCR of clone MC.27.759S according to the invention minus the N-terminal leader sequence and minus the constant region (i.e. the α chain variable region).

SEQ ID NO: 15 is the sequence of the native β chain of the TCR of clone MC.27.759S according to the invention minus the N-terminal leader sequence and minus the constant region (i.e. the β chain variable region).

SEQ ID NO: 16 is the codon-optimised DNA sequence encoding a TCR comprising the α and β chains connected by a linker (see Example 8)

SEQ ID NO: 17 is the sequence of a linker (see Example 9)

SEQ ID NO: 18 is a primer sequence (see Example 2)

SEQ ID NO: 19 is a primer sequence (see Example 2)

SEQ ID NO: 20 is the sequence of a purification tag.

SEQ ID NO: 21 is the sequence of a purification tag.

SEQ ID NO: 22 is the sequence of a purification tag.

SEQ ID NO: 23 is the sequence of a Melan-A derived peptide (see Example 4)

SEQ ID NO: 24 is the sequence of a P2A cleavable linker

SEQ ID NO: 25 is the sequence of a E2A cleavable linker

SEQ ID NO: 26 is the sequence of a F2A cleavable linker

SEQ ID NO: 27 is the sequence of a T2A cleavable linker

SEQ ID NO: 28 is the sequence of a linker

SEQ ID NO: 29 is the sequence of the native α chain of the TCR of clone MC.27.759S according to the invention minus the N-terminal leader sequence.

SEQ ID NO: 30 is the sequence of a fusion protein encoding a TCR comprising the α and β chains connected by a linker (see Example 8)

SEQ ID NO: 31 is the sequence of the native β chain of the TCR of clone MC.27.759S according to the invention minus the N-terminal leader sequence

SEQ ID NO: 32 is the sequence of the TRAJ42 portion of CDR3α

SEQ ID NO: 33 is CDR3α of clone A3, A4 and C2

SEQ ID NO: 34 is CDR3α of clone C1

SEQ ID NO: 35 is CDR3α of variant clone A1 with TRAVJ 42+TRAV 41 recombination

SEQ ID NO: 36 CDR3β of clone A3

SEQ ID NO: 37 CDR3β of clone A4

SEQ ID NO: 38 CDR3β of clones C2 and C3

SEQ ID NO: 39 CDR1α of clones A3, A4 and C2, TRAV9-2

SEQ ID NO: 40 is CDR2α of clone A3, A4 and C2, TRAV9-2

SEQ ID NO: 41 CDR1α of clone C1, TRAV41

SEQ ID NO: 42 is CDR2α of clone C1, TRAV41

SEQ ID NO: 43 CDR1β of clones C1 and C2, TRBV28

SEQ ID NO: 44 CDR2β of clones C1 and C2, TRBV28

SEQ ID NO: 45 is Variable-α of clone A3 (CDRs underlined), TRAV9-2, TRAJ 42

SEQ ID NO: 46 Variable-β of clone A3 (CDRs underlined), TRBV 12-4, TRBJ 2-2

SEQ ID NO: 47 is Variable-α of clone A4 (CDRs underlined), TRAV9-2, TRAJ 42

SEQ ID NO: 48 Variable-β of clone A4 (CDRs underlined), TRBV 12-4, TRBJ 1-5

SEQ ID NO: 49 is Variable-α of clone C1 (CDRs underlined), TRAV41, TRAJ 42

SEQ ID NO: 50 Variable-β of clone C1 (CDRs underlined), TRBV 28, TRBJ 2-5

SEQ ID NO: 51 is Variable-α of clone C2 (CDRs underlined), TRAV9-2, TRAJ 42

SEQ ID NO: 52 Variable-β of clone C2 (CDRs underlined), TRBV 28, TRBJ 2-5 SEQ ID NO: 54 beta chain extracellular region A3 (TRBV 12-4, TRBJ 2-2)

SEQ ID NO: 53 alpha chain extracellular region A3 (TRAV9-2, TRAJ 42), (CDRs underlined)

SEQ ID NO: 54 beta chain extracellular region A3 (TRBV 12-4, TRBJ 2-2), (CDRs underlined)

SEQ ID NO: 55 alpha chain extracellular region A4 (TRAV9-2, TRAJ 42), (CDRs underlined)

NO: 56 beta chain extracellular region A4 (TRBV 12-4, TRBJ 1-5), (CDRs underlined)

SEQ ID NO: 57 alpha chain extracellular region C1 (TRAV41, TRAJ 42), (CDRs underlined)

SEQ ID NO: 58 beta chain extracellular region C1 (TRBV 28, TRBJ 2-5), (CDRs underlined)

SEQ ID NO: 59 alpha chain extracellular region C2 (TRAV9-2, TRAJ 42), (CDRs underlined)

SEQ ID NO: 60 beta chain extracellular region C2 (TRBV 28, TRBJ 2-5), (CDRs underlined)

N.B. sequence italics denote constant region sequences.

### DETAILED DESCRIPTION OF THE INVENTION

### Definitions

Suitably, the polypeptides and polynucleotides used in the present invention are isolated. An “isolated” polypeptide or polynucleotide is one that is removed from its original environment. For example, a naturally occurring polypeptide or polynucleotide is isolated if it is separated from some or all of the coexisting materials in the natural system. A polynucleotide is considered to be isolated if, for example, it is cloned into a vector that is not a part of its natural environment.

“Naturally occurring” or “native”, which terms are interchangeable, when used with reference to a polypeptide or polynucleotide sequence means a sequence found in nature and not synthetically modified.

The term “artificial” when used with reference to a polypeptide or polynucleotide sequence means a sequence not found in nature which is, for example, a synthetic modification of a natural sequence, or contains an unnatural polypeptide or polynucleotide sequence.

The term “engineered” when used with reference to a cell means a cell not found in nature which is, for example, a synthetic modification of a natural cell, for example, because it contains or expresses foreign elements and/or lacks natural elements.

The term “heterologous” when used with reference to the relationship of one polynucleotide or polypeptide to another polynucleotide or polypeptide indicates that the two or more sequences are not found in the same relationship to each other in nature.

The term “heterologous” when used with reference to the relationship of one polynucleotide or polypeptide sequence to a cell means a sequence which is not isolated from, derived from, expressed by said cell or associated with or based upon a naturally occurring polynucleotide or polypeptide sequence found in the said cell.

The term “domain”, when used with reference to a TCR, is generally used to refer to a part of the TCR formed of the corresponding region of the two chains. For example, the transmembrane regions of the α and β chains form the transmembrane domain.

The term “intracellular” domain or region is used interchangeably with the term “cytoplasmic” domain or region and in the literature this is sometimes referred to as the “cytosolic” domain or region.

### T-Cell Receptor (TCR)

A TCR of this invention comprises an α chain and a β chain. The extracellular region of each chain comprises three CDRs (CDR1, CDR2, CDR3) and four framework regions which are either side of the CDRs, and a constant region. The soluble form of the TCR consists of the extracellular domain (also called the antigen-binding domain). An entire TCR (as opposed to the soluble form) has the extracellular domain as well as, at its C terminus, transmembrane and intracellular domains. An immature form of an entire TCR also comprises a leader peptide sequence at its N terminus which is removed after translation by cellular peptidases such as signal peptidase. Each chain has a connecting peptide region which links the transmembrane and intracellular regions to the extracellular domain at its C terminus.

In a preferred embodiment of the invention there is provided a cancer-specific T-cell receptor (TCR) or a cancer-specific binding fragment of a TCR which binds a tumour antigen, wherein the TCR or binding fragment comprises a variable region of an α chain which is encoded by a sequence resulting from the recombination of a human T Cell Receptor Alpha Variable (TRAV) gene and human T cell Receptor Alpha Joining Gene TRAJ42, optionally the TRAV gene may be any one of TRAV21, TRAV41, TRAV 9-2, preferably the TCR or binding fragment comprises an alpha chain complementarity-determining region 3 (CDR3α) which comprises the amino acid sequence YGGSQGNLIF (SEQ ID NO: 32).

In a preferred embodiment of the invention, said α chain CDR3 comprises or consists of CAVRLAGYGGSQGNLIF (SEQ ID NO: 1), CALSSYHYGGSQGNLIF (SEQ ID NO: 33), CAVREADYGGSQGNLIF (SEQ ID NO: 34) or CAVRFGYYGGSQGNLIF (SEQ ID NO: 35) or a variant CDR3 that has 1, 2, 3, 4 or 5 amino acid variations or 1, 2, 3 or 4 or 1, 2 or 3 amino acid variations selected from additions, substitutions and deletions with respect thereto, such as 1 or 2 variations, such as 1 variation. In a preferred embodiment of the invention, said β chain CDR3 comprises or consists of CASSSQGTDTQYF (SEQ ID NO: 2), CASRGNTGELFF (SEQ ID NO: 36), CASRTGQGNQPQHF (SEQ ID NO: 37), or CASSLEQGQYF (SEQ ID NO: 38) or a variant CDR3 that has 1, 2, 3, 4 or 5 amino acid variations or 1, 2, 3 or 4 or 1, 2 or 3 amino acid variations selected from additions, substitutions and deletions with respect thereto, such as 1 or 2 variations, such as 1 variation.

Cancer-specific binding fragments of the said TCR and the respective α and β chains are also provided.

Thus, an embodiment of the invention provides a cancer-specific T-cell receptor (TCR) or an α chain thereof or a cancer-specific binding fragment of a TCR which binds a tumour antigen comprising:
a CDR3α comprising or consisting of CAVRLAGYGGSQGNLIF (SEQ ID NO: 1), CALSSYHYGGSQGNLIF (SEQ ID NO: 33), CAVREADYGGSQGNLIF (SEQ ID NO: 34) or CAVRFGYYGGSQGNLIF (SEQ ID NO: 35) or a variant CDR3α that has 1, 2, 3, 4 or 5 amino acid variations or 1, 2, 3 or 4 or 1, 2 or 3 amino acid variations selected from additions, substitutions and deletions with respect thereto, such as 1, 2 or 3 variations or 1 or 2 variations, such as 1 variation.

An embodiment of the invention also provides a cancer-specific T-cell receptor (TCR) or a β chain thereof or a cancer-specific binding fragment of a TCR which binds a tumour antigen comprising:
a CDR3β comprising or consisting of CASSSQGTDTQYF (SEQ ID NO: 2), CASRGNTGELFF (SEQ ID NO: 36), CASRTGQGNQPQHF (SEQ ID NO: 37), or CASSLEQGQYF (SEQ ID NO: 38) or a variant CDR3β that has 1, 2, 3, 4 or 5 amino acid variations or 1, 2, 3 or 4 or 1, 2 or 3 amino acid variations selected from additions, substitutions and deletions with respect thereto, such as 1 or 2 variations, such as 1 variation.

A further embodiment of the invention provides tumour-specific T-cell receptor (TCR) or tumour-specific binding fragment of a TCR as above wherein the TCR or tumour-specific binding fragment comprises:
a CDR3α comprising or consisting of CAVRLAGYGGSQGNLIF (SEQ ID NO: 1), CALSSYHYGGSQGNLIF (SEQ ID NO: 33), CAVREADYGGSQGNLIF (SEQ ID NO: 34) or CAVRFGYYGGSQGNLIF (SEQ ID NO: 35) or a variant CDR3α that has 1, 2, 3, 4 or 5 variations or 1, 2, 3 or 4 or 1, 2 or 3 amino acid variations selected from additions, substitutions and deletions with respect thereto, such as 1 or 2 variations, such as 1 variation; and
a CDR3β comprising or consisting of CASSSQGTDTQYF (SEQ ID NO: 2), CASRGNTGELFF (SEQ ID NO: 36), CASRTGQGNQPQHF (SEQ ID NO: 37), or CASSLEQGQYF (SEQ ID NO: 38) or a variant CDR3β that has 1, 2, 3, 4 or 5 amino acid variations or 1, 2, 3 or 4 or 1, 2 or 3 amino acid variations selected from additions, substitutions and deletions with respect thereto, such as 1 or 2 variations, such as 1 variation.

A further embodiment of the invention provides a cancer-specific T-cell receptor (TCR) or a cancer-specific binding fragment of a TCR as above comprising:
(a) a CDR3α comprising or consisting of CAVRLAGYGGSQGNLIF, (SEQ ID NO: 1) and/or a CDR3β comprising or consisting of CASSSQGTDTQYF, (SEQ ID NO:2),
(b) a CDR3α comprising or consisting of CALSSYHYGGSQGNLIF (SEQ ID NO: 33) and/or a CDR3β comprising or consisting of CASRGNTGELFF (SEQ ID NO: 36),
(c) a CDR3α comprising or consisting of CALSSYHYGGSQGNLIF (SEQ ID NO: 33) and/or a CDR3β comprising or consisting of CASRTGQGNQPQHF (SEQ ID NO: 37),
(d) a CDR3α comprising or consisting of CAVREADYGGSQGNLIF (SEQ ID NO: 34) and/or a CDR3β comprising or consisting of CASSLEQGQYF (SEQ ID NO: 38), or
(e) a CDR3α comprising or consisting of CALSSYHYGGSQGNLIF (SEQ ID NO: 33) and/or a CDR3β comprising or consisting of CASSLEQGQYF (SEQ ID NO: 38).

A further embodiment of the invention provides a cancer-specific T-cell receptor (TCR) or a cancer-specific binding fragment of a TCR as above comprising:
(a) a CDR3α comprising or consisting of CAVRLAGYGGSQGNLIF, (SEQ ID NO: 1) and a CDR3β comprising or consisting of CASSSQGTDTQYF, (SEQ ID NO:2),
(b) a CDR3α comprising or consisting of CALSSYHYGGSQGNLIF (SEQ ID NO: 33) and a CDR3β comprising or consisting of CASRGNTGELFF (SEQ ID NO: 36),
(c) a CDR3α comprising or consisting of CALSSYHYGGSQGNLIF (SEQ ID NO: 33) and a CDR3β comprising or consisting of CASRTGQGNQPQHF (SEQ ID NO: 37),
(d) a CDR3α comprising or consisting of CAVREADYGGSQGNLIF (SEQ ID NO: 34) and a CDR3β comprising or consisting of CASSLEQGQYF (SEQ ID NO: 38), or
(e) a CDR3α comprising or consisting of CALSSYHYGGSQGNLIF (SEQ ID NO: 33) and a CDR3β comprising or consisting of CASSLEQGQYF (SEQ ID NO: 38).

The CDRs described herein represent the CDR3s of said TCR which are the main CDRs responsible for recognizing processed and presented antigen or ligand. The other CDRs (CDR1α, CDR2α, CDR1β and CDR2β) are encoded by the germline. Therefore, the invention further concerns a TCR also including one or more of these other CDRs i.e. CDR1α, CDR2α, CDR1ß or CDR2β.

Accordingly, in a preferred embodiment there is provided a TCR or cancer-specific binding fragment of a TCR wherein the TCR or cancer-specific binding fragment comprises:
(a) one or more, including any combination, of the following CDRs comprising or consisting of; DSAIYN, (SEQ ID NO: 3), IQSSQREQ, (SEQ ID NO: 4), SGHDY, (SEQ ID NO: 5), and FNNNVP, (SEQ ID NO: 6), or a variant CDR that has 1 or 2 amino acid variations, such as 1 variation with respect thereto,
(b) one or more, including any combination, of the following CDRs comprising or consisting of; ATGYPS, (SEQ ID NO: 39), ATKADDK, (SEQ ID NO: 40), SGHDY, (SEQ ID NO: 5), and FNNNVP, (SEQ ID NO: 6), or a variant CDR that has 1 or 2 amino acid variations, such as 1 variation with respect thereto,
(c) one or more, including any combination, of the following CDRs comprising or consisting of; ATGYPS, (SEQ ID NO: 39), ATKADDK, (SEQ ID NO: 40), SGHDY, (SEQ ID NO: 5), and FNNNVP, (SEQ ID NO: 6), or a variant CDR that has 1 or 2 amino acid variations, such as 1 variation with respect thereto
(d) one or more, including any combination, of the following CDRs comprising or consisting of; VGISA, (SEQ ID NO: 41), LSSGK, (SEQ ID NO: 42), MDHEN, (SEQ ID NO: 43), and SYDVKM, (SEQ ID NO: 44), or a variant CDR that has 1 or 2 amino acid variations, such as 1 variation with respect thereto; or
(e) one or more, including any combination, of the following CDRs comprising or consisting of; ATGYPS, (SEQ ID NO: 39), ATKADDK, (SEQ ID NO: 40), MDHEN, (SEQ ID NO: 43), and SYDVKM, (SEQ ID NO: 44); or a variant CDR that has 1 or 2 amino acid variations, such as 1 variation with respect thereto; wherein in each case the variations are selected from additions, substitutions and deletions.

Reference herein to a cancer-specific TCR is to a TCR that specifically recognises a tumour cell or a tumour-cell ligand, in the context of MR1, e.g. tumour-cell antigen or ligand presented by MR1 or in complex with MR1 for example at the tumour cell surface, and is activated by same but is not activated by a non-tumour cell or a non-tumour-cell ligand, in the context of MR1.

In a preferred embodiment of the invention said TCR is an αβ TCR having an α chain and a β chain and said CDR of said α chain and said β chain comprises or consists of
(a) the CDR CAVRLAGYGGSQGNLIF, (SEQ ID NO: 1) and the CDR CASSSQGTDTQYF, (SEQ ID NO:2),
(b) the CDR CALSSYHYGGSQGNLIF (SEQ ID NO: 33) and the CDR CASRGNTGELFF (SEQ ID NO: 36),
(c) the CDR CALSSYHYGGSQGNLIF (SEQ ID NO: 33) and the CDR CASRTGQGNQPQHF (SEQ ID NO: 37),
(d) the CDR CAVREADYGGSQGNLIF (SEQ ID NO: 34) and the CDR CASSLEQGQYF (SEQ ID NO: 38), or
(e) the CDR CALSSYHYGGSQGNLIF (SEQ ID NO: 33) and the CDR CASSLEQGQYF (SEQ ID NO: 38); or in each case a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations, such as 1 variation.

Accordingly, said TCR may comprise one or both of the aforementioned CDRs and in a preferred embodiment comprises both of said CDRs.

In an embodiment, the cancer-specific T-cell receptor (TCR) or a cancer-specific binding fragment of a TCR comprises a chain, for example α-chain, with CDRs comprising or consisting of:
(a) CAVRLAGYGGSQGNLIF, (SEQ ID NO: 1) or a variant CDR that has 1, 2, 3, 4 or 5 amino acid variations with respect thereto; and DSAIYN, (SEQ ID NO: 3); and IQSSQREQ, (SEQ ID NO: 4) or a variant CDR that has 1 or 2 amino acid variations with respect thereto, such as 1 variation;
(b) CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation,
(c) CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation,
(d) CAVREADYGGSQGNLIF (SEQ ID NO: 34) or a variant CDR that has 1, 2, 3, 4 or 5 amino acid variations with respect thereto; and VGISA, (SEQ ID NO: 41) and LSSGK, (SEQ ID NO: 42) or a variant CDR that has 1 or 2 amino acid variations with respect thereto, such as 1 variation; or
(e) CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation; wherein in each case the variations are selected from additions, substitutions and deletions.

For example, the cancer-specific T-cell receptor (TCR) or a cancer-specific binding fragment of a TCR comprises a chain, for example an α-chain, with CDRs comprising or consisting of: (a) CAVRLAGYGGSQGNLIF, (SEQ ID NO: 1), DSAIYN, (SEQ ID NO: 3); and IQSSQREQ, (SEQ ID NO: 4); (b) CALSSYHYGGSQGNLIF (SEQ ID NO: 33), ATGYPS, (SEQ ID NO: 39) and ATKADDK, (SEQ ID NO: 40), (c) CALSSYHYGGSQGNLIF (SEQ ID NO: 33), ATGYPS, (SEQ ID NO: 39) and ATKADDK, (SEQ ID NO: 40), (d) CAVREADYGGSQGNLIF (SEQ ID NO: 34), VGISA, (SEQ ID NO: 41) and LSSGK, (SEQ ID NO: 42); or (e) CALSSYHYGGSQGNLIF (SEQ ID NO: 33), ATGYPS, (SEQ ID NO: 39) and ATKADDK, (SEQ ID NO: 40).

In an embodiment, the cancer-specific T-cell receptor (TCR) or a cancer-specific binding fragment of a TCR comprises a chain, for example a β-chain, with CDRs comprising or consisting of:
(a) CASSSQGTDTQYF, (SEQ ID NO: 2) or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation,
(b) CASRGNTGELFF (SEQ ID NO: 36), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation,
(c) CASRTGQGNQPQHF (SEQ ID NO: 37), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation,
(d) CASSLEQGQYF (SEQ ID NO: 38), or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations, such as 1 variation; or
(e) CASSLEQGQYF (SEQ ID NO: 38) or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; wherein in each case the variations are selected from additions, substitutions and deletions.

For example, the cancer-specific T-cell receptor (TCR) or a cancer-specific binding fragment of a TCR comprises a chain, for example a β-chain with CDRs comprising or consisting of: (a) CASSSQGTDTQYF, (SEQ ID NO: 2), SGHDY (SEQ ID NO: 5) and FNNNVP, (SEQ ID NO: 6), (b) CASRGNTGELFF (SEQ ID NO: 36), SGHDY (SEQ ID NO: 5) and FNNNVP, (SEQ ID NO: 6), (c) CASRTGQGNQPQHF (SEQ ID NO: 37), SGHDY (SEQ ID NO: 5) and FNNNVP, (SEQ ID NO: 6), (d) CASSLEQGQYF (SEQ ID NO: 38), MDHEN (SEQ ID NO: 43) and SYDVKM, (SEQ ID NO: 44), or (e) CASSLEQGQYF (SEQ ID NO: 38), MDHEN (SEQ ID NO: 43) and SYDVKM, (SEQ ID NO: 44).

In an embodiment, the cancer-specific T-cell receptor (TCR) or cancer-specific binding fragment of a TCR comprises 3 α chain CDRs comprising or consisting of:
(a) 3 α-chain CDRs comprising or consisting of CAVRLAGYGGSQGNLIF, (SEQ ID NO: 1) or a variant CDR that has 1, 2, 3, 4 or 5 amino acid variations with respect thereto; DSAIYN, (SEQ ID NO: 3) and IQSSQREQ, (SEQ ID NO: 4) or a variant CDR that has 1 or 2 amino acid variations with respect thereto, such as 1 variation; and comprises 3 β-chain CDRs comprising or consisting of CASSSQGTDTQYF, (SEQ ID NO: 2) or a variant CDR that has 1, 2, 3, 4 or 5 amino acid variations with respect thereto, and SGHDY, (SEQ ID NO: 5) and FNNNVP, (SEQ ID NO: 6) or a variant CDR that has 1 or 2 amino acid variations with respect thereto, such as 1 variation,
(b) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRGNTGELFF (SEQ ID NO: 36), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation;
(c) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRTGQGNQPQHF (SEQ ID NO: 37), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation;
(d) 3 α-chain CDRs comprising or consisting of CAVREADYGGSQGNLIF (SEQ ID NO: 34) or a variant CDR that has 1, 2, 3, 4 or 5 amino acid variations with respect thereto; and VGISA, (SEQ ID NO: 41) and LSSGK, (SEQ ID NO: 42) or a variant CDR that has 1 or 2 amino acid variations with respect thereto, such as 1 variation; and comprises 3-β chain CDRs comprising or consisting of CASSLEQGQYF (SEQ ID NO: 38), or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; or
(e) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation and comprises 3-β chain CDRs comprising or consisting of CASSLEQGQYF (SEQ ID NO: 38) or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; wherein in each case the variations are selected from additions, substitutions and deletions.

For example, the cancer-specific T-cell receptor (TCR) or cancer-specific binding fragment of a TCR comprises 3 α-chain CDRs and 3 β-chain CDRs comprising or consisting of: comprising or consisting of: (a) 3 α-chain CDRs comprising or consisting of CAVRLAGYGGSQGNLIF, (SEQ ID NO: 1), DSAIYN, (SEQ ID NO: 3) and IQSSQREQ, (SEQ ID NO: 4); and 3 β-chain CDRs comprising or consisting of CASSSQGTDTQYF, (SEQ ID NO: 2), SGHDY, (SEQ ID NO: 5) and FNNNVP, (SEQ ID NO: 6), (b) 3 α-chain CDRs comprising or consisting of CALSSYHYGGSQGNLIF (SEQ ID NO: 33), ATGYPS, (SEQ ID NO: 39) and ATKADDK, (SEQ ID NO: 40), and 3 β-chain CDRs comprising or consisting of CASRGNTGELFF (SEQ ID NO: 36), SGHDY (SEQ ID NO: 5) and FNNNVP, (SEQ ID NO: 6); (c) 3 α-chain CDRs comprising or consisting of CALSSYHYGGSQGNLIF (SEQ ID NO: 33), ATGYPS, (SEQ ID NO: 39) and ATKADDK, (SEQ ID NO: 40), and 3 β-chain CDRs comprising or consisting of CASRTGQGNQPQHF (SEQ ID NO: 37), SGHDY (SEQ ID NO: 5) and FNNNVP, (SEQ ID NO: 6); (d) 3 α-chain CDRs comprising or consisting of CAVREADYGGSQGNLIF (SEQ ID NO: 34), (SEQ ID NO: 41) and LSSGK, (SEQ ID NO: 42); and 3 β-chain CDRs comprising or consisting of CASSLEQGQYF (SEQ ID NO: 38), MDHEN (SEQ ID NO: 43) and SYDVKM, or (e) 3 α-chain CDRs comprising or consisting of CALSSYHYGGSQGNLIF (SEQ ID NO: 33), ATGYPS, (SEQ ID NO: 39) and ATKADDK, (SEQ ID NO: 40), and 3 β-chain CDRs comprising or consisting of CASSLEQGQYF (SEQ ID NO: 38), MDHEN (SEQ ID NO: 43) and SYDVKM, (SEQ ID NO: 44).

As noted TCRs according to the present invention is unconventional in that it is not HLA-restricted, rather it binds to a cancer-specific ligand in the context of MR1, an alternative MHC-related molecule, e.g. a cancer-specific ligand presented by MR1 or in complex with MR1 for example at the tumour cell surface. We demonstrate herein that a further type of MR1-restricted T-cells exists that does not express the semi-invariant MAIT TCR α chains, moreover, advantageously, these T-cells and their TCRs are tumour-specific (i.e. respond to tumour cells but not non-tumour cells) but, surprisingly, are able to identify many tumour types of different origin or tissue type and so have pan-cancer therapy potential. Further, the fact that these T-cells and their TCRs are not HLA-restricted means they have pan-population therapy potential and so represent an extremely important new cancer therapy.

The specific mechanism that allows killing of many types of cancer cells is still to be elucidated. Nevertheless, it may be hypothesized (and without being limited by theory) that the T-cell and associated TCR of the invention is able to bind to surface-displayed MR1 molecules liganded with a by-product (or by-products) of an aberrant cancer-specific metabolic pathway(s) (for example, an altered pathway arising from epigenetic changes associated with neoplastic transformation), with the type of aberrant metabolism being a common feature amongst different types of cancer.

The sequence of the α chain of the native TCR of the MC.27.759S clone is as follows:

(SEQ ID NO: 7)
METLLGLLILWLQLQWVSSKQEVTQIPAALSVPEGENLVLNCSFT
DSAIYNLQWFRQDPGKGLTSLLLIQSSQREQTSGRLNASLDKSSG
RSTLYIAASQPGDSATYLCAVRLAGYGGSQGNLIFGKGTKLSVKP
NIQNPDPAVYQLRDSKSSDKSVCLFTDFDSQTNVSQSKDSDVYIT
DKTVLDMRSMDFKSNSAVAWSNKSDFACANAFNNSIIPEDTFFPS
PESSCDVKLVEKSFETDTNLNFQNLSVIGFRILLLKVAGFNLLMT
LRLWSS.

The variable region of the α chain of the native TCR of the MC.27.759S clone is encoded by recombination of genes TRAV21 and TRAJ42. In SEQ ID NO: 7, residues 1-19 are the N-terminal leader sequence (shown in underlined normal type), residues 20-45 are framework region 1, residues 46-51 are CDR1 (shown in underlined bold type), residues 52-68 are framework region 2, residues 69-76 are CDR2 (shown in underlined bold type), residues 77-108 are framework region 3, residues 109-125 are CDR3 (shown in underlined bold type), residues 126-135 are framework region 4 and residues 136-276 are the constant region (shown in italics type). Within the constant region, residues 227-256 are the connecting peptide region, residues 257-273 are the transmembrane region and residues 274-276 are the intracellular region.

The leader sequence is removed after translation of the protein in the host cell and is not part of the receptor when incorporated into a cell membrane.

The corresponding α chain of the native TCR of the MC.27.759S clone minus the N-terminal leader sequence is provided as SEQ ID NO: 29:

(SEQ ID NO: 29)
KQEVTQIPAALSVPEGENLVLNCSFTDSAIYNLQWFRQDPGKGLTSL
LLIQSSQREQTSGRLNASLDKSSGRSTLYIAASQPGDSATYLCAVRL
AGYGGSQGNLIFGKGTKLSVKPNIQNPDPAVYQLRDSKSSDKSVCLF
TDFDSQTNVSQSKDSDVYITDKTVLDMRSMDFKSNSAVAWSNKSDFA
CANAFNNSIIPEDTFFPSPESSCDVKLVEKSFETDTNLNFQNLSVIG
FRILLLKVAGFNLLMTLRLWSS.

The corresponding α chain of the native TCR of the MC.27.759S clone minus the N-terminal leader sequence and the constant region (i.e. the α chain variable region) is provided as SEQ ID NO: 14:

(SEQ ID NO: 14)
KQEVTQIPAALSVPEGENLVLNCSFTDSAIYNLQWFRQDPGKGLTSL
LLIQSSQREQTSGRLNASLDKSSGRSTLYIAASQPGDSATYLCAVRL
AGYGGSQGNLIFGKGTKLSVKP.

The extracellular region of this native α chain is as follows:

(SEQ ID NO: 9)
KQEVTQIPAALSVPEGENLVLNCSFTDSAIYNLQWFRQDPGKGLTSL
LLIQSSQREQTSGRLNASLDKSSGRSTLYIAASQPGDSATYLCAVRL
AGYGGSQGNLIFGKGTKLSVKPNIQNPDPAVYQLRDSKSSDKSVCLF
TDFDSQTNVSQSKDSDVYITDKTVLDMRSMDFKSNSAVAWSNKSDFA
CANAFNNSIIPEDTFFPSPESSCDVKLVEKSFETDTNLNFQNLSVIG
FR.

In SEQ ID NO: 9, residues 1-26 are framework region 1, residues 27-32 are CDR1 (shown in underlined bold type), residues 33-49 are framework region 2, residues 50-57 are CDR2 (shown in underlined bold type), residues 58-89 are framework region 3, residues 90-106 are CDR3 (shown in underlined bold type), residues 107-116 are framework region 4 and residues 117-237 are the constant region (shown in italics type).

The corresponding alpha chain variable region and extracellular domain (minus N-terminal leader sequence) is provided for the clones A3, A4, C1 and C2 (framework normal capitals, CDRs underlined and bold, constant region italic):

V-alpha A3 (TRAV9-2, TRAJ 42)
SEQ ID NO: 45
DSVTQMEGPVTLSEEAFLTINCTYTATGYPSLFWYVQYPGEGLQLLL
KATKADDKGSNKGFEATYRKETTSFHLEKGSVQVSDSAVYFCALSSY
HYGGSQGNLIFGKGTKLSVKP,.
V-alpha A4 (TRAV9-2, TRAJ 42)
SEQ ID NO: 47
DSVTQMEGPVTLSEEAFLTINCTYTATGYPSLFWYVQYPGEGLQLLL
KATKADDKGSNKGFEATYRKETTSFHLEKGSVQVSDSAVYFCALSSY
HYGGSQGNLIFGKGTKLSVKP,.
V-alpha C1 (TRAV41, TRAJ 42)
SEQ ID NO: 49
NEVEQSPQNLTAQEGEFITINCSYSVGISALHWLQQHPGGGIVSLFM
LSSGKKKHGRLIATINIQEKHSSLHITASHPRDSAVYICAVRLAGYG
GSQGNLIFGKGTKLSVKP,.
V-alpha C2 (TRAV9-2, TRAJ 42)
SEQ ID NO: 51
DSVTQMEGPVTLSEEAFLTINCTYTATGYPSLFWYVQYPGEGLQLLL
KATKADDKGSNKGFEATYRKETTSFHLEKGSVQVSDSAVYFCALSSY
HYGGSQGNLIFGKGTKLSVKP,.
alpha chain extracellular region A3 (TRAV9-2,
TRAJ 42)
SEQ ID NO: 53
DSVTQMEGPVTLSEEAFLTINCTYTATGYPSLFWYVQYPGEGLQLLL
KATKADDKGSNKGFEATYRKETTSFHLEKGSVQVSDSAVYFCALSSY
HYGGSQGNLIFGKGTKLSVKPNIQNPDPAVYQLRDSKSSDKSVCLFT
DFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGF
R,.
alpha chain extracellular region A4 (TRAV9-2,
TRAJ 42)
SEQ ID NO: 55
DSVTQMEGPVTLSEEAFLTINCTYTATGYPSLFWYVQYPGEGLQLLL
KATKADDKGSNKGFEATYRKETTSFHLEKGSVQVSDSAVYFCALSSY
HYGGSQGNLIFGKGTKLSVKPNIQNPDPAVYQLRDSKSSDKSVCLFT
DFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGF
R,.
alpha chain extracellular region C1 (TRAV41,
TRAJ 42)
SEQ ID NO: 57
NEVEQSPQNLTAQEGEFITINCSYSVGISALHWLQQHPGGGIVSLFM
LSSGKKKHGRLIATINIQEKHSSLHITASHPRDSAVYICAVRLAGYG
GSQGNLIFGKGTKLSVKPNIQNPDPAVYQLRDSKSSDKSVCLFTDFD
SQTNVSQSKDSDVYITDKTVLDMRSMDFKSNSAVAWSNKSDFACANA
FNNSIIPEDTFFPSPESSCDVKLVEKSFETDTNLNFQNLSVIGFR,.
alpha chain extracellular region C2 (TRAV9-2,
TRAJ 42)
SEQ ID NO: 59
DSVTQMEGPVTLSEEAFLTINCTYTATGYPSLFWYVQYPGEGLQLLL
KATKADDKGSNKGFEATYRKETTSFHLEKGSVQVSDSAVYFCALSSY
HYGGSQGNLIFGKGTKLSVKPNIQNPDPAVYQLRDSKSSDKSVCLFT
DFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGF
R,.
The sequence of the β chain of the native TCR
of the MC.27.759S clone is as follows:
(SEQ ID NO: 8)
MGSWTLCCVSLCILVAKHTDAGVIQSPRHEVTEMGQEVTLRCKPISG
HDYLFWYRQTMMRGLELLIYFNNNVPIDDSGMPEDRFSAKMPNASFS
TLKIQPSEPRDSAVYFCASSSQGTDTQYFGPGTRLTVLEDLKNVFPP
KVAVFEPSEAEISHTQKATLVCLATGFYPDHVELSWWVNGKEVHSGV
STDPQPLKEQPALNDSRYCLSSRLRVSATFWQNPRNHFRCQVQFYGL
SENDEWTQDRAKPVTQIVSAEAWGRADCGFTSESYQQGVLSATILYE
ILLGKATLYAVLVSALVLMAMVKRKDSRG.

The variable region of the β chain of the TCR of the native MC.27.759S clone is encoded by recombination of genes TRBV9 and TRDB2 and TRBJ2.3

In SEQ ID NO: 8, residues 1-19 are the N-terminal leader sequence (shown in underlined normal type), residues 20-45 are framework region 1, residues 46-50 are CDR1 (shown in underlined bold type), residues 51-67 are framework region 2, residues 68-73 are CDR2 (shown in underlined bold type), residues 74-110 are framework region 3, residues 111-123 are CDR3 (shown in underlined bold type), residues 124-132 are framework region 4 and residues 133-311 are the constant region (shown in italics type). Within the constant region, residues 262-282 are the connecting peptide region, residues 283-304 are the transmembrane region and residues 305-311 are the intracellular region.

The leader sequence is removed after translation of the protein in the host cell and is not part of the receptor when incorporated into a cell membrane.

The corresponding β chain of the native TCR of the MC.27.759S clone minus the N-terminal leader sequence is provided as SEQ ID NO: 31:

(SEQ ID NO: 31)
DAGVIQSPRHEVTEMGQEVTLRCKPISGHDYLFWYRQTMMRGLELLI
YFNNNVPIDDSGMPEDRFSAKMPNASFSTLKIQPSEPRDSAVYFCAS
SSQGTDTQYFGPGTRLTVLEDLKNVFPPKVAVFEPSEAEISHTQKAT
LVCLATGFYPDHVELSWWVNGKEVHSGVSTDPQPLKEQPALNDSRYC
LSSRLRVSATFWQNPRNHFRCQVQFYGLSENDEWTQDRAKPVTQIVS
AEAWGRADCGFTSESYQQGVLSATILYEILLGKATLYAVLVSALVLM
AMVKRKDSRG.

The corresponding β chain of the native TCR of the MC.27.759S clone minus the N-terminal leader sequence and the constant region (i.e. the β chain variable region) is provided as SEQ ID NO: 15:

(SEQ ID NO: 15)
DAGVIQSPRHEVTEMGQEVTLRCKPISGHDYLFWYRQTMMRGLELLI
YFNNNVPIDDSGMPEDRFSAKMPNASFSTLKIQPSEPRDSAVYFCAS
SSQGTDTQYFGPGTRLTVL.

The extracellular region of this native β chain is as follows:

(SEQ ID NO: 10)
DAGVIQSPRHEVTEMGQEVTLRCKPISGHDYLFWYRQTMMRGLELLI
YFNNNVPIDDSGMPEDRFSAKMPNASFSTLKIQPSEPRDSAVYFCAS
SSQGTDTQYFGPGTRLTVLEDLKNVFPPKVAVFEPSEAEISHTQKAT
LVCLATGFYPDHVELSWWVNGKEVHSGVSTDPQPLKEQPALNDSRYC
LSSRLRVSATFWQNPRNHFRCQVQFYGLSENDEWTQDRAKPVTQIVS
AEAWGRADCGFTSESYQQGVLSATILYE.

In SEQ ID NO: 10, residues 1-26 are framework region 1, residues 27-31 are CDR1 (shown in underlined bold type), residues 32-48 are framework region 2, residues 49-54 are CDR2 (shown in underlined bold type), residues 55-91 are framework region 3, residues 92-104 are CDR3 (shown in underlined bold type), residues 105-114 are framework region 4 and residues 115-263 are the constant region (show in italics type).

The corresponding beta chain variable region and extracellular domain (minus the N-terminal leader sequence) is provided for the clones A3, A4, C1 and C2 (framework normal capitals, CDRs underlined, constant region italic):

V-beta A3 (TRBV 12-4, TRBJ 2-2)
SEQ ID NO: 46
GVIQSPRHEVTEMGQEVTLRCKPISGHDYLFWYRQTMMRGLELLIYF
NNNVPIDDSGMPEDRFSAKMPNASFSTLKIQPSEPRDSAVYFCASSL
CASRGNTGELFFGEGSRLTVL,.
V-beta A4 (TRBV 12-4, TRBJ 1-5)
SEQ ID NO: 48
GVIQSPRHEVTEMGQEVTLRCKPISGHDYLFWYRQTMMRGLELLIYF
NNNVPIDDSGMPEDRFSAKMPNASFSTLKIQPSEPRDSAVYFCASSL
CASRTGQGNQPQHFGDGTRLSIL,.
V-beta C1 (TRBV 28, TRBJ 2-5)
SEQ ID NO: 50
SRYLVKRTGEKVFLECVQDMDHENMFWYRQDPGLGLRLIYFSYDVKM
KEKGDIPEGYSVSREKKERFSLILESASTNQTSMYLCASSLCASSLE
QGQYFGPGTRLLVL,.
V-beta C2 (TRBV 28, TRBJ 2-5)
SEQ ID NO: 52
SRYLVKRTGEKVFLECVQDMDHENMFWYRQDPGLGLRLIYFSYDVKM
KEKGDIPEGYSVSREKKERFSLILESASTNQTSMYLCASSLEQGQYF
GPGTRLLVL,.
beta chain extracellular region A3 (TRBV 12-4,
TRBJ 2-2)
SEQ ID NO: 54
GVIQSPRHEVTEMGQEVTLRCKPISGHDYLFWYRQTMMRGLELLIYF
NNNVPIDDSGMPEDRFSAKMPNASFSTLKIQPSEPRDSAVYFCASSL
CASRGNTGELFFGEGSRLTVLEDLKNVFPPKVAVFEPSEAEISHTQK
ATLVCLATGFYPDHVELSWWVNGKEVHSGVSTDPQPLKEQPALNDSR
YCLSSRLRVSATFWQNPRNHFRCQVQFYGLSENDEWTQDRAKPVTQI
VSAEAWGRADCGFTSESYQQGVLSATILYE,.
beta chain extracellular region A4 (TRBV 12-4,
TRBJ 1-5)
SEQ ID NO: 56
GVIQSPRHEVTEMGQEVTLRCKPISGHDYLFWYRQTMMRGLELLIYF
NNNVPIDDSGMPEDRFSAKMPNASFSTLKIQPSEPRDSAVYFCASSL
CASRTGQGNQPQHFGDGTRLSILEDLKNVFPPKVAVFEPSEAEISHT
QKATLVCLATGFYPDHVELSWWVNGKEVHSGVSTDPQPLKEQPALND
SRYCLSSRLRVSATFWQNPRNHFRCQVQFYGLSENDEWTQDRAKPVT
QIVSAEAWGRADCGFTSESYQQGVLSATILYE,.
beta chain extracellular region C1 (TRBV 28,
TRBJ 2-5)
SEQ ID NO: 58
SRYLVKRTGEKVFLECVQDMDHENMFWYRQDPGLGLRLIYFSYDVKM
KEKGDIPEGYSVSREKKERFSLILESASTNQTSMYLCASSLCASSLE
QGQYFGPGTRLLVLEDLKNVFPPKVAVFEPSEAEISHTQKATLVCLA
TGFYPDHVELSWWVNGKEVHSGVSTDPQPLKEQPALNDSRYCLSSRL
RVSATFWQNPRNHFRCQVQFYGLSENDEWTQDRAKPVTQIVSAEAWG
RADCGFTSESYQQGVLSATILYE,.
beta chain extracellular region C2 (TRBV 28,
TRBJ 2-5)
SEQ ID NO: 60
SRYLVKRTGEKVFLECVQDMDHENMFWYRQDPGLGLRLIYFSYDVKM
KEKGDIPEGYSVSREKKERFSLILESASTNQTSMYLCASSLEQGQYF
GPGTRLLVLEDLKNVFPPKVAVFEPSEAEISHTQKATLVCLATGFYP
DHVELSWWVNGKEVHSGVSTDPQPLKEQPALNDSRYCLSSRLRVSAT
FWQNPRNHFRCQVQFYGLSENDEWTQDRAKPVTQIVSAEAWGRADCG
FTSESYQQGVLSATILYE,.

In a further preferred embodiment of the invention said TCR α chain is a mutant TCR α chain and comprises or consists of:

KQEVTQIPAALSVPEGENLVLNCSFTDSAIYNLQWFRQDPGKGLTSLLLIQSSQREQTS GRLNASLDKSSGRSTLYIAASQPGDSATYLCAVRLAGYGGSQGNLIEGKGTKLSVKPN IQNPDPAVYQLRDSKSSDKSVCLFTDFDSQTNVSQSKDSDVYITDKCVLDMRSMDFKS NSAVAWSNKSDFACANAFNNSIIPEDTFFPSPESSCDVKLVEKSFETDTNLNFQNLSVI GFR. (SEQ ID NO: 11) or a variant sequence that has at least 85% identity therewith, such as 86%, 87%, 88%, 89%, 90%, 91%, 92%, 93%, 94%, 95%, 96%, 97%, 98% or 99%.

In SEQ ID NO: 11, residues 1-26 are framework region 1, residues 27-32 are CDR1 (shown in underlined bold type), residues 33-49 are framework region 2, residues 50-57 are CDR2 (shown in underlined bold type), residues 58-89 are framework region 3, residues 90-106 are CDR3 (shown in underlined bold type), residues 107-116 are framework region 4 and residues 117-237 are the constant region (shown in italics type). SEQ ID NO: 11 defines the extracellular region of the mutant TCR α chain. SEQ ID NO: 11 is the same as SEQ ID NO: 9 except for residue 164 (underlined) which is C in SEQ ID NO: 11 and T in SEQ ID NO: 9.

In a further preferred embodiment of the invention said TCR β chain is a mutant TCR β chain and comprises or consists of:

DAGVIQSPRHEVTEMGQEVTLRCKPISGHDYLFWYRQTMMRGLELLIYFNNNVPIDDS GMPEDRFSAKMPNASFSTLKIQPSEPRDSAVYFCASSSQGTDTQYEGPGTRLTVLED LKNVFPPKVAVFEPSEAEISHTQKATLVCLATGFYPDHVELSWWVNGKEVHSGVCTDP QPLKEQPALNDSRYALSSRLRVSATFWQDPRNHFRCQVQFYGLSENDEWTQDRAKP VTQIVSAEAWGRADCGFTSESYQQGVLSATILYE. (SEQ ID NO: 12) or a variant sequence that has at least 85% identity therewith, such as 86%, 87%, 88%, 89%, 90%, 91%, 92%, 93%, 94%, 95%, 96%, 97%, 98% or 99%.

In SEQ ID NO: 12, residues 1-26 are framework region 1, residues 27-31 are CDR1 (shown in underlined bold type), residues 32-48 are framework region 2, residues 49-54 are CDR2 (shown in underlined bold type), residues 55-91 are framework region 3, residues 92-104 are CDR3 (shown in underlined bold type), residues 105-114 are framework region 4 and residues 115-263 are the constant region (show in italics type). SEQ ID NO: 12 defines the extracellular region of the mutant TCR β chain.

SEQ ID NO: 12 is the same as SEQ ID NO: 10 except for (i) residue 170 (underlined) which is C in SEQ ID NO: 12 and S in SEQ ID NO: 10; (ii) and residue 188 (underlined) which is A in SEQ ID NO: 12 and C in SEQ ID NO: 10 (iii) residue 202 (underlined) which is D in SEQ ID NO: 12 and N in SEQ ID NO: 10.

In a further preferred embodiment of the invention said TCR β chain is a mutant TCR β chain and comprises or consists of:

DAGVIQSPRHEVTEMGQEVTLRCKPISGHDYLFWYRQTMMRGLELLIYFNNNVPIDDS GMPEDRFSAKMPNASFSTLKIQPSEPRDSAVYFCASSSQGTDTQYFGPGTRLTVLED LKNVFPPKVAVFEPSEAEISHTQKATLVCLATGFYPDHVELSWWVNGKEVHSGVCTDP QPLKEQPALNDSRYCLSSRLRVSATFWQNPRNHFRCQVQFYGLSENDEWTQDRAKP VTQIVSAEAWGRADCGFTSESYQQGVLSATILYE. (SEQ ID NO: 13), or a variant sequence that has at least 85% identity therewith, such as 86%, 87%, 88%, 89%, 90%, 91%, 92%, 93%, 94%, 95%, 96%, 97%, 98% or 99%.

In SEQ ID NO: 13, residues 1-26 are framework region 1, residues 27-31 are CDR1 (shown in underlined bold type), residues 32-48 are framework region 2, residues 49-54 are CDR2 (shown in underlined bold type), residues 55-91 are framework region 3, residues 92-104 are CDR3 (shown in underlined bold type), residues 105-113 are framework region 4 and residues 114-263 are the constant region (show in italics type). SEQ ID NO: 13 defines the extracellular region of the mutant TCR β chain.

SEQ ID NO: 13 is the same as SEQ ID NO: 10 except for residue 170 (underlined) which is C in SEQ ID NO: 13 and S in SEQ ID NO: 10.

In an embodiment, the cancer-specific T-cell receptor (TCR) or cancer-specific binding fragment of a TCR comprises an α chain extracellular region comprising or consisting of SEQ ID NO: 9; or a variant α chain extracellular region which has at least 85% sequence identity to the α chain extracellular region of SEQ ID NO: 9 (e.g. at least 86%, 87%, 88%, 89%, 90%, 91%, 92%, 93%, 94%, 95%, 96%, 97%, 98% or 99% sequence identity therewith).

An example of a variant of SEQ ID NO: 9 is SEQ ID NO: 11.

In an embodiment, the cancer-specific T-cell receptor (TCR) or cancer-specific binding fragment of a TCR comprises an α chain extracellular region comprising or consisting of SEQ ID NO: 11; or a variant α chain extracellular region which has at least 85% sequence identity to the α chain extracellular region of SEQ ID NO: 11 (e.g. at least 86%, 87%, 88%, 89%, 90%, 91%, 92%, 93%, 94%, 95%, 96%, 97%, 98% or 99% sequence identity therewith).

An example of a variant of SEQ ID NO: 11 is SEQ ID NO: 9.

In an embodiment, the cancer-specific T-cell receptor (TCR) or cancer-specific binding fragment of a TCR comprises an α chain extracellular region comprising or consisting of SEQ ID NO: 9.

In an embodiment, the cancer-specific T-cell receptor (TCR) or cancer-specific binding fragment of a TCR comprises an α chain extracellular region comprising or consisting of SEQ ID NO: 11.

In an embodiment, the cancer-specific T-cell receptor (TCR) or cancer-specific binding fragment of a TCR comprises a β chain extracellular region comprising or consisting of SEQ ID NO: 10; or a variant β chain extracellular region which has at least 85% sequence identity to the β chain extracellular region of SEQ ID NO: 10 (e.g. at least 86%, 87%, 88%, 89%, 90%, 91%, 92%, 93%, 94%, 95%, 96%, 97%, 98% or 99% sequence identity therewith).

An example of a variant of SEQ ID NO: 10 is SEQ ID NO: 12 and SEQ ID NO: 13.

In an embodiment, the cancer-specific T-cell receptor (TCR) or cancer-specific binding fragment of a TCR comprises a β chain extracellular region comprising or consisting of SEQ ID NO: 12; or a variant β chain extracellular region which has at least 85% sequence identity to the β chain extracellular region of SEQ ID NO: 12 (e.g. at least 86%, 87%, 88%, 89%, 90%, 91%, 92%, 93%, 94%, 95%, 96%, 97%, 98% or 99% sequence identity therewith).

An example of a variant of SEQ ID NO: 12 is SEQ ID NO: 10 and SEQ ID NO: 13.

In an embodiment, the cancer-specific T-cell receptor (TCR) or cancer-specific binding fragment of a TCR comprises a β chain extracellular region comprising or consisting of SEQ ID NO: 13; or a variant β chain extracellular region which has at least 85% sequence identity to the β chain extracellular region of SEQ ID NO: 13 (e.g. at least 86%, 87%, 88%, 89%, 90%, 91%, 92%, 93%, 94%, 95%, 96%, 97%, 98% or 99% sequence identity therewith).

An example of a variant of SEQ ID NO: 13 is SEQ ID NO: 10 and SEQ ID NO: 12.

In an embodiment, the cancer-specific T-cell receptor (TCR) or cancer-specific binding fragment of a TCR comprises a β chain extracellular region comprising or consisting of SEQ ID NO: 10.

In an embodiment, the cancer-specific T-cell receptor (TCR) or cancer-specific binding fragment of a TCR comprises a β chain extracellular region comprising or consisting of SEQ ID NO: 12.

In an embodiment, the cancer-specific T-cell receptor (TCR) or cancer-specific binding fragment of a TCR comprises a β chain extracellular region comprising or consisting of SEQ ID NO: 13.

The invention further provides, a TCR or cancer-specific binding fragment of a TCR, wherein the TCR comprises an α chain extracellular region comprising or consisting of any of SEQ ID NO: 9, 53, 55, 57 or 59, or a variant α chain extracellular region which has at least 85% sequence identity to the α chain extracellular region of SEQ ID NO: 9, 53, 55, 57 or 59, such as at least 85% identity therewith, for example 86%, 87%, 88%, 89%, 90%, 91%, 92%, 93%, 94%, 95%, 96%, 97%, 98% or 99% and/or comprises a β chain extracellular region comprising or consisting of any of SEQ ID NO: 10, 54, 56, 57 or 60, or a variant β chain extracellular region which has at least 85% sequence identity to the α chain extracellular region of SEQ ID NO: 9, 53, 55, 57 or 59, such as at least 85% identity therewith, for example 86%, 87%, 88%, 89%, 90%, 91%, 92%, 93%, 94%, 95%, 96%, 97%, 98% or 99%.

In a preferred embodiment the TCR or cancer-specific binding fragment of a TCR of the invention comprises an α and β chain extracellular region respectively comprising or consisting of any of (a) SEQ ID NO: 9 and 10, (b) SEQ ID NO: 53 and 54, (c) SEQ ID NO: 55 and 56, (d) SEQ ID NO: 57 and 58, or (e) SEQ ID NO: 59 and 60 or in either or both chains a variant extracellular region which has at least 85% sequence identity to the respective extracellular region, such as at least 85% identity therewith, for example 86%, 87%, 88%, 89%, 90%, 91%, 92%, 93%, 94%, 95%, 96%, 97%, 98% or 99%.

The invention further provides, a TCR or cancer-specific binding fragment of a TCR, wherein the TCR comprises an α chain variable region comprising or consisting of any of SEQ ID NO: 14, 45, 47, 49, or 51, or a variant α chain variable region which has at least 85% sequence identity to the α chain variable region of SEQ ID NO: 14, 45, 47, 49, or 51, such as at least 85% identity therewith, for example 86%, 87%, 88%, 89%, 90%, 91%, 92%, 93%, 94%, 95%, 96%, 97%, 98% or 99% and/or comprises a β chain variable region comprising or consisting of any of SEQ ID NO: 15, 46, 48, 50, or 52, or a variant β chain variable region which has at least 85% sequence identity to the α chain variable region of SEQ ID NO: 15, 46, 48, 50, or 52, such as at least 85% identity therewith, for example 86%, 87%, 88%, 89%, 90%, 91%, 92%, 93%, 94%, 95%, 96%, 97%, 98% or 99%.

In a preferred embodiment the TCR or cancer-specific binding fragment of a TCR of the invention comprises an α and β chain variable region respectively comprising or consisting of any of (a) SEQ ID NO: 14 and 15, (b) SEQ ID NO: 45 and 46, (c) SEQ ID NO: 47 and 48, (d) SEQ ID NO: 49 and 50, or (e) SEQ ID NO: 51 and 52 or in either or both chains a variant variable region which has at least 85% sequence identity to the respective variable region, such as at least 85% identity therewith, for example 86%, 87%, 88%, 89%, 90%, 91%, 92%, 93%, 94%, 95%, 96%, 97%, 98% or 99%.

In yet a further preferred embodiment of the invention said TCR comprises said aforementioned TCR α chain and said aforementioned TCR β chain.

Suitably, the TCR of the invention comprises:
(a) an α chain comprising a CDR3 sequence of SEQ ID NO: 1 or a variant CDR3 that has 1, 2 or 3 amino acid variations with respect thereto, such as 1 or 2 variations such as 1 variation, a CDR1 sequence of SEQ ID NO:3 or a variant CDR1 that has 1 or 2 amino acid variations with respect thereto, such as 1 variation and CDR2 sequence of SEQ ID NO: 4 or a variant CDR2 that has 1 or 2 amino acid variations with respect thereto, such as 1 variation and a β chain comprising a CDR3 sequence of SEQ ID NO: 2 or a variant CDR3 that has 1, 2 or 3 amino acid variations with respect thereto, such as 1 or 2 variations such as 1 variation, a CDR1 sequence of SEQ ID NO: 5 or a variant CDR1 that has 1 or 2 amino acid variations with respect thereto, such as 1 variation and CDR2 sequence of SEQ ID NO: 6 or a variant CDR2 that has 1 or 2 amino acid variations with respect thereto, such as 1 variation; or
(b) an α chain extracellular region comprising or consisting of SEQ ID NO: 11 or a variant α chain extracellular region which has at least 85% sequence identity to the α chain extracellular region of SEQ ID NO: 11 and a β chain extracellular region comprising SEQ ID NO: 12 or a variant β chain extracellular region which has at least 85% sequence identity to the β chain extracellular region of SEQ ID NO: 12 or a β chain extracellular region comprising SEQ ID NO: 13 or a variant β chain extracellular region which has at least 85% sequence identity to the β chain extracellular region of SEQ ID NO: 13; or
(c) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising or consisting of SEQ ID NO: 10 or a variant β chain extracellular region which has at least 85% sequence identity to the β chain extracellular region of SEQ ID NO: 10;
(d) an α chain comprising or consisting of SEQ ID NO: 29 or a variant α chain which has at least 85% sequence identity to the α chain of SEQ ID NO: 29 and a β chain comprising or consisting of SEQ ID NO: 31 or a variant β chain which has at least 85% sequence identity to the β chain of SEQ ID NO: 31; or
(e) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising SEQ ID NO: 12 or a variant β chain extracellular region which has at least 85% sequence identity to the β chain extracellular region of SEQ ID NO: 12 or a β chain extracellular region comprising SEQ ID NO: 13 or a variant β chain extracellular region which has at least 85% sequence identity to the β chain extracellular region of SEQ ID NO: 13; or
(f) an α chain variable region comprising or consisting of SEQ ID NO: 14 or a variant α chain variable region which has at least 85% sequence identity to the α chain variable region of SEQ ID NO: 14 and a β chain variable region comprising or consisting of SEQ ID NO: 15 or a variant β chain variable region which has at least 85% sequence identity to the β chain variable region of SEQ ID NO: 15; or
(g) an α chain variable region comprising or consisting of SEQ ID NO: 45 or a variant α chain variable region which has at least 85% sequence identity to the α chain variable region of SEQ ID NO: 45 and a β chain variable region comprising or consisting of SEQ ID NO: 46 or a variant β chain variable region which has at least 85% sequence identity to the β chain variable region of SEQ ID NO: 46; or
(h) an α chain variable region comprising or consisting of SEQ ID NO: 47 or a variant α chain variable region which has at least 85% sequence identity to the α chain variable region of SEQ ID NO: 47 and a β chain variable region comprising or consisting of SEQ ID NO: 48 or a variant β chain variable region which has at least 85% sequence identity to the β chain variable region of SEQ ID NO: 48; or
(i) an α chain variable region comprising or consisting of SEQ ID NO: 49 or a variant α chain variable region which has at least 85% sequence identity to the α chain variable region of SEQ ID NO: 49 and a β chain variable region comprising or consisting of SEQ ID NO: 50 or a variant β chain variable region which has at least 85% sequence identity to the β chain variable region of SEQ ID NO: 50; or
(j) an α chain variable region comprising or consisting of SEQ ID NO: 51 or a variant α chain variable region which has at least 85% sequence identity to the α chain variable region of SEQ ID NO: 51 and a β chain variable region comprising or consisting of SEQ ID NO: 52 or a variant β chain variable region which has at least 85% sequence identity to the β chain variable region of SEQ ID NO: 52; optionally wherein in each case the variations are selected from additions, substitutions and deletions.

The variable region of the α chain of the native TCR of the MC.27.759S clone is encoded by recombination of genes TRAV21 and TRAJ42 (Table 1). For reference the MR1 restricted TCR of T-cell clone MC.7.G5, see WO2019/097244; referred to as “MC.7.G5”, has the chain usage TRBV25-1, TRBJ2-3, TRAV38-2/DV8, TRAJ31 MC.7.G5. The present invention provides, a cancer-specific T-cell receptor (TCR) or a cancer-specific binding fragment of a TCR which binds a tumour antigen and which may comprise a variable region of the α chain which is encoded by a sequence resulting from the recombination of a human T Cell Receptor Alpha Variable (TRAV) gene and human T cell Receptor Alpha Joining Gene TRAJ42, for example the TRAV gene may be any one of TRAV21, TRAV41, or TRAV 9-2. The foregoing recombination results in the formation of an alpha chain complementarity-determining region 3 (CDR3α) which comprises the amino acid sequence YGGSQGNLIF (SEQ ID NO: 32). Consequently, according to the present invention, the cancer-specific T-cell receptor (TCR) or a cancer-specific binding fragment of a TCR which binds a tumour antigen may comprise an alpha chain complementarity-determining region 3 (CDR3α) comprising the sequence YGGSQGNLIF (SEQ ID NO: 32). Accordingly, the cancer-specific T-cell receptor (TCR) or a cancer-specific binding fragment of a TCR which binds a tumour antigen according to the invention may comprise an alpha chain complementarity-determining region 3 (CDR3α) comprising or consisting of a sequence selected from any one of CAVRLAGYGGSQGNLIF (SEQ ID NO: 1), CALSSYHYGGSQGNLIF (SEQ ID NO: 33), CAVREADYGGSQGNLIF (SEQ ID NO: 34) or CAVRFGYYGGSQGNLIF (SEQ ID NO: 35) or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation.

According to the present invention the cancer-specific T-cell receptor (TCR) or a cancer-specific binding fragment of a TCR which binds a tumour antigen comprises an alpha chain:
(a) encoded by recombination of genes TRAV9-2 and TRAJ42, and comprising an alpha chain complementarity-determining region 3 (CDR3α) comprising the sequence YGGSQGNLIF (SEQ ID NO: 32), and/or the sequence comprising or consisting of CALSSYHYGGSQGNLIF (SEQ ID NO: 33), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation; or
(b) encoded by recombination of genes TRAV41 and TRAJ42, and comprising an alpha chain complementarity-determining region 3 (CDR3α) comprising the sequence YGGSQGNLIF (SEQ ID NO: 32), and/or the sequence comprising or consisting of CAVREADYGGSQGNLIF (SEQ ID NO: 34), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation; or
(c) encoded by recombination of genes TRAV41 and TRAJ42, and comprising an alpha chain complementarity-determining region 3 (CDR3α) comprising the sequence YGGSQGNLIF (SEQ ID NO: 32), and/or the sequence comprising or consisting of CAVRFGYYGGSQGNLIF (SEQ ID NO: 35), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation.

The invention further provides a tumour-specific T-cell receptor (TCR) or tumour-specific binding fragment of a TCR wherein the TCR or tumour-specific binding fragment comprises:
(i) a CDR3α comprising or consisting of CALSSYHYGGSQGNLIF (SEQ ID NO: 33) or a variant CDR3α that has 1, 2, 3, 4 or 5 variations or 1, 2, 3 or 4 or 1, 2 or 3 amino acid variations selected from additions, substitutions and deletions with respect thereto, such as 1 or 2 variations, such as 1 variation; and/or a CDR3β comprising or consisting of CASRGNTGELFF (SEQ ID NO: 36) or a variant CDR3β that has 1, 2, 3, 4 or 5 amino acid variations or 1, 2, 3 or 4 or 1, 2 or 3 amino acid variations selected from additions, substitutions and deletions with respect thereto, such as 1 or 2 variations, such as 1 variation, or
(ii) a CDR3α comprising or consisting of CALSSYHYGGSQGNLIF (SEQ ID NO: 33) or a variant CDR3α that has 1, 2, 3, 4 or 5 variations or 1, 2, 3 or 4 or 1, 2 or 3 amino acid variations selected from additions, substitutions and deletions with respect thereto, such as 1 or 2 variations, such as 1 variation; and/or a CDR3β comprising or consisting of CASRTGQGNQPQHF (SEQ ID NO: 37) or a variant CDR3β that has 1, 2, 3, 4 or 5 amino acid variations or 1, 2, 3 or 4 or 1, 2 or 3 amino acid variations selected from additions, substitutions and deletions with respect thereto, such as 1 or 2 variations, such as 1 variation, or
(iii) a CDR3α comprising or consisting of CAVREADYGGSQGNLIF (SEQ ID NO: 34) or a variant CDR3α that has 1, 2, 3, 4 or 5 variations or 1, 2, 3 or 4 or 1, 2 or 3 amino acid variations selected from additions, substitutions and deletions with respect thereto, such as 1 or 2 variations, such as 1 variation; and/or a CDR3β comprising or consisting of CASSLEQGQYF (SEQ ID NO: 38) or a variant CDR3β that has 1, 2, 3, 4 or 5 amino acid variations or 1, 2, 3 or 4 or 1, 2 or 3 amino acid variations selected from additions, substitutions and deletions with respect thereto, such as 1 or 2 variations, such as 1 variation, or
(iv) a CDR3α comprising or consisting of CALSSYHYGGSQGNLIF (SEQ ID NO: 33) or a variant CDR3α that has 1, 2, 3, 4 or 5 variations or 1, 2, 3 or 4 or 1, 2 or 3 amino acid variations selected from additions, substitutions and deletions with respect thereto, such as 1 or 2 variations, such as 1 variation; and/or a CDR3β comprising or consisting of CASSLEQGQYF (SEQ ID NO: 38) or a variant CDR3β that has 1, 2, 3, 4 or 5 amino acid variations or 1, 2, 3 or 4 or 1, 2 or 3 amino acid variations selected from additions, substitutions and deletions with respect thereto, such as 1 or 2 variations, such as 1 variation.

According to the present invention there is provided a cancer-specific T-cell receptor (TCR) or a cancer-specific binding fragment of a TCR which binds a tumour antigen which comprises:
(i) an alpha chain encoded by recombination of genes TRAV9-2 and TRAJ42, and comprising an alpha chain complementarity-determining region 3 (CDR3α) comprising the sequence YGGSQGNLIF (SEQ ID NO: 32), and/or comprising or consisting of the sequence CALSSYHYGGSQGNLIF (SEQ ID NO: 33), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation and/or a beta chain encoded by recombination of genes TRBV12-4 and TRBJ2-2, and comprising a beta chain complementarity-determining region 3 (CDR3β) comprising or consisting of the sequence CASRGNTGELFF (SEQ ID NO: 36), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation, or
(ii) an alpha chain encoded by recombination of genes TRAV9-2 and TRAJ42, and comprising an alpha chain complementarity-determining region 3 (CDR3α) comprising the sequence YGGSQGNLIF (SEQ ID NO: 32), and/or comprising or consisting of the sequence CALSSYHYGGSQGNLIF (SEQ ID NO: 33), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation and/or a beta chain encoded by recombination of genes TRBV12-4 and TRBJ1-5, and comprising a beta chain complementarity-determining region 3 (CDR3β) comprising or consisting of the sequence CASRTGQGNQPQHF (SEQ ID NO: 37), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation, or
(iii) an alpha chain encoded by recombination of genes TRAV41 and TRAJ42, and comprising an alpha chain complementarity-determining region 3 (CDR3α) comprising the sequence YGGSQGNLIF (SEQ ID NO: 32), and/or comprising or consisting of the sequence CAVREADYGGSQGNLIF (SEQ ID NO: 34), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation and/or a beta chain encoded by recombination of genes TRBV28 and TRBJ2-5, and comprising a beta chain complementarity-determining region 3 (CDR3β) comprising or consisting of the sequence CASSLEQGQYF (SEQ ID NO: 38), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation, or
(iv) an alpha chain encoded by recombination of genes TRAV9-2 and TRAJ42, and comprising an alpha chain complementarity-determining region 3 (CDR3α) comprising the sequence YGGSQGNLIF (SEQ ID NO: 32), and/or comprising or consisting of the sequence CALSSYHYGGSQGNLIF (SEQ ID NO: 33), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation and/or a beta chain encoded by recombination of genes TRBV28 and TRBJ2-5, and comprising a beta chain complementarity-determining region 3 (CDR3β) comprising or consisting of the sequence CASSLEQGQYF (SEQ ID NO: 38), or sequence or a variant CDR that has 1, 2, 3, 4 or 5 amino acid variations or 1, 2, 3 or 4 or 1, 2 or 3 amino acid variations selected from additions, substitutions and deletions with respect thereto, such as 1 or 2 variations such as 1 variation.

According to the present invention there is provided a cancer-specific T-cell receptor (TCR) or a cancer-specific binding fragment of a TCR which binds a tumour antigen which comprises:
(i) an alpha chain encoded by recombination of genes TRAV9-2 and TRAJ42, and comprising an alpha chain complementarity-determining region 3 (CDR3α) comprising or consisting of the sequence CALSSYHYGGSQGNLIF (SEQ ID NO: 33), and/or a beta chain encoded by recombination of genes TRBV12-4 and TRBJ2-2, and comprising a beta chain complementarity-determining region 3 (CDR3β) comprising or consisting of the sequence CASRGNTGELFF (SEQ ID NO: 36), or
(ii) an alpha chain encoded by recombination of genes TRAV9-2 and TRAJ42, and comprising an alpha chain complementarity-determining region 3 (CDR3α) comprising or consisting of the sequence CALSSYHYGGSQGNLIF (SEQ ID NO: 33), and/or a beta chain encoded by recombination of genes TRBV12-4 and TRBJ1-5, and comprising a beta chain complementarity-determining region 3 (CDR3β) comprising or consisting of the sequence CASRTGQGNQPQHF (SEQ ID NO: 37), or
(iii) an alpha chain encoded by recombination of genes TRAV41 and TRAJ42, and comprising or consisting of the sequence CAVREADYGGSQGNLIF (SEQ ID NO: 34), and/or a beta chain encoded by recombination of genes TRBV28 and TRBJ2-5, and comprising a beta chain complementarity-determining region 3 (CDR3β) comprising or consisting of the sequence CASSLEQGQYF (SEQ ID NO: 38), or
(iv) an alpha chain encoded by recombination of genes TRAV9-2 and TRAJ42, and comprising an alpha chain complementarity-determining region 3 (CDR3α) comprising or consisting of the sequence CALSSYHYGGSQGNLIF (SEQ ID NO: 33), and/or a beta chain encoded by recombination of genes TRBV28 and TRBJ2-5, and comprising a beta chain complementarity-determining region 3 (CDR3β) comprising or consisting of the sequence CASSLEQGQYF (SEQ ID NO: 38);
or a variant sequence that has at least 85% identity therewith, such as 86%, 87%, 88%, 89%, 90%, 91%, 92%, 93%, 94%, 95%, 96%, 97%, 98%, 99% or 100%.

Examples of a cancer-specific T-cell receptor (TCR) or a cancer-specific binding fragment of a TCR which binds a tumour antigen according to the invention are set out in Table 1 below, said TCRs bind tumour antigen and T-cells presenting the tabulated TCRs recognise and are activated by cancer cells/tumour cells as shown in the examples herein.

TABLE 1

| TCR | | CDR3 | | | CDR3 | |
|---|---|---|---|---|---|---|
| Clone | TRVB | beta | TRBJ | TRAV | alpha | TRAJ |
| A3 | TRBV12- | CASRGN | TRBJ2- | TRAV9- | CALSSYHYG | TRAJ42 |
| | 4 | TGELFF | 2 | 2 | GSQGNLIF | |
| | | SEQ ID | | | SEQ ID | |
| | | NO: 36 | | | NO: 33 | |
| A4 | TRBV12- | CASRTGQ | TRBJ1- | TRAV9- | CALSSYHYG | TRAJ42 |
| | 4 | GNQPQHF | 5 | 2 | GSQGNLIF | |
| | | SEQ ID | | | SEQ ID | |
| | | NO: 37 | | | NO: 33 | |
| C1 | TRBV28 | CASSLE | TRBJ2- | TRAV41 | CAVREADYG | TRAJ42 |
| | | QGQYF | 5 | | GSQGNLIF | |
| | | SEQ ID | | | SEQ ID | |
| | | NO: 38 | | | NO: 34 | |
| C2 | TRBV28 | CASSLE | TRBJ2- | TRAV9- | CALSSYHYG | TRAJ 42 |
| | | QGQYF | 5 | 2 | GSQGNLIF | |
| | | SEQ ID | | | SEQ ID | |
| | | NO: 38 | | | NO: 33 | |
| MC.27. | TRBV12- | CASSSQG | TRBJ2- | TRAV21 | CAVRLAGYG | TRAJ42 |
| 759S | 4 | TDTQYF | 3 | | GSQGNLIF | |
| | | (SEQ ID | | | (SEQ ID | |
| | | NO: 2) | | | NO: 1) | |

In an embodiment, the TCR or cancer-specific binding fragment thereof is MR1-restricted.

In yet another preferred embodiment of the invention said TCR is part of a chimeric receptor having the functionality described herein.

TCR polypeptide sequences of the invention can be obtained and manipulated using the techniques disclosed for example in Green and Sambrook 2012 Molecular Cloning: A Laboratory Manual 4th Edition Cold Spring Harbour Laboratory Press.

### Sequence Comparisons

For the purposes of comparing two closely related polypeptide sequences, the “% sequence identity” between a first sequence and a second sequence may be calculated. Polypeptide sequences are said to be the same as or identical to other polypeptide sequences, if they share 100% sequence identity over their entire length. Residues in sequences are numbered from left to right, i.e. from N- to C-terminus for polypeptides. The terms “identical” or percentage “identity”, in the context of two or more polypeptide sequences, refer to two or more sequences or sub-sequences that are the same or have a specified percentage of amino acid residues that are the same (i.e. at least 85%, 86%, 87%, 88%, 89%, 90%, 91%, 92%, 93%, 94%, 95%, 96%, 97%, 98% or 99% sequence identity over a specified region), when compared and aligned for maximum correspondence over a comparison window. Suitably, the comparison is performed over a window corresponding to the entire length of the reference sequence.

For sequence comparison, one sequence acts as the reference sequence, to which the test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percentage sequence identities for the test sequences relative to the reference sequence, based on the program parameters.

A “comparison window”, as used herein, refers to a segment in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerised implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

An example of an algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. Software for performing BLAST analyses is publicly available through the National Center for Biotechnology Information (website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance.

A “difference” between sequences refers to an insertion, deletion or substitution of a single residue in a position of the second sequence, compared to the first sequence. Two sequences can contain one, two or more such differences. Insertions, deletions or substitutions in a second sequence which is otherwise identical (100% sequence identity) to a first sequence result in reduced % sequence identity. For example, if the identical sequences are 9 residues long, one substitution in the second sequence results in a sequence identity of 88.9%. If the identical sequences are 17 amino acid residues long, two substitutions in the second sequence results in a sequence identity of 88.2%.

Alternatively, for the purposes of comparing a first, reference sequence to a second, comparison sequence, the number of additions, substitutions and/or deletions made to the first sequence to produce the second sequence may be ascertained. An addition is the addition of one residue into the first sequence (including addition at either terminus of the first sequence). A substitution is the substitution of one residue in the first sequence with one different residue. A deletion is the deletion of one residue from the first sequence (including deletion at either terminus of the first sequence).

### Sequence Variants

The term “amino acid” refers to any one of the naturally occurring amino acids, as well as amino acid analogues and amino acid mimetics that function in a manner which is similar to the naturally occurring amino acids. Naturally occurring amino acids are those 20 L-amino acids encoded by the genetic code, as well as those amino acids that are later modified, e.g., hydroxyproline, γ-carboxyglutamate, and O-phosphoserine. The term “amino acid analogue” refers to a compound that has the same basic chemical structure as a naturally occurring amino acid, i.e., an α carbon that is bound to a hydrogen, a carboxyl group, an amino group, and an R group but has a modified R group or a modified peptide backbone as compared with a natural amino acid. Examples include homoserine, norleucine, methionine sulfoxide, methionine methyl sulfonium and norleucine. Amino acid mimetics refers to chemical compounds that have a structure that is different from the general chemical structure of an amino acid, but that functions in a manner similar to a naturally occurring amino acid. Suitably an amino acid is a naturally occurring amino acid or an amino acid analogue, especially a naturally occurring amino acid and in particular one of those 20 L-amino acids encoded by the genetic code.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In an embodiment, the amino acid sequence of the TCR or cancer-specific binding fragment thereof is artificial. Without limitation, and as discussed further herein, the TCR or cancer-specific binding fragment thereof may comprise at least one mutation to remove a cysteine residue by replacement with another residue and/or to introduce a cysteine residue by replacement of another residue with cysteine.

In an embodiment, at least one amino acid is substituted, added or deleted relative to the wildtype sequence.

In an embodiment, the at least one amino acid is (are) located in a framework region, a CDR or a constant region, particularly in a framework region or a constant region, especially in a constant region. Thus, particularly, any and all additions, substitutions and deletions of amino acids are in a framework region or a constant region, particularly in a constant region.

In an embodiment, in respect of variation in the sequences of any of SEQ ID NOs: 7-15, 29-31, 42-60, preferably the at least one amino acid is not located in any CDR.

Variations in sequence can be in the form of amino acid additions, substitutions and deletions, especially substitutions. For example, additions can be at the N and/or C termini of sequences and deletions can be at the N and/or C termini of sequences. There may, for example, be 1, 2 or 3 (such as 1 or 2, e.g. 1) amino acid variations selected from additions, substitutions and deletions in the sequence of SEQ ID NO: 1, 33, 34 or 35. There may, for example, be 1, 2 or 3 (such as 1 or 2, e.g. 1) amino acid variations selected from additions, substitutions and deletions in the sequence of SEQ ID NO: 2, 36, 37 or 38. There may, for example, be 1 or 2 (such as 1) amino acid variations selected from additions, substitutions and deletions in the sequences of any of SEQ ID NOs: 3-6, 39-44. There may, for example, be up to 24 e.g. up to 20 e.g. up to 15 e.g. up to 10 e.g. up to 5 e.g. 4, 3, 2 or 1) amino acid variations selected from additions, substitutions and deletions in the sequences of SEQ ID NOs: 7-13, 29, 31, 42-60. There may, for example, be up to 15 e.g. up to 10 e.g. up to 5 e.g. 4, 3, 2 or 1 amino acid variations selected from additions, substitutions and deletions in the sequences of SEQ ID NOs: 29, 30 or 31.

Substitutions are suitably conservative substitutions. The following eight groups each contain amino acids that are typically conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins 1984).

Suitably, sequence variations do not significantly adversely affect the ability of the TCR or fragment thereof to bind to its target epitope on the tumour, for example its binding affinity of the variant is 75% or more e.g. 80% or more e.g. 85% or more e.g. 90% or more e.g. 95% or more e.g. 98% or more e.g. 99% or more of that of the TCR that in soluble form has α and β chains respectively of any of (a) SEQ ID NOs: 9 and 10, (b) SEQ ID NOs: 53 and 54, (c) SEQ ID NOs: 55 and 56, (d) SEQ ID NOs: 57 and 58, or (e) SEQ ID NOs: 99 and 60.

Where sequence variants (e.g. of an α or β chain or a fragment thereof e.g. cancer specific binding fragment, variable domain, a soluble fragment or cancer specific binding fragment, or a CDR) are said to have at least 85% identity to a reference sequence, they may have at least 86%, 87%, 88%, 89%, 90%, 91%, 92%, 93%, 94%, 95%, 96%, 97%, 98% or 99% sequence identity to the reference sequence.

The following variants formed of sequence mutations are contemplated in particular:

Particularly useful mutations are mutations made to one or both chains (i.e. one or both of the α and β chains) of a TCR of the invention to reduce the likelihood that said chain or chains will pair with α chain or chains of the TCR naturally produced by a T-cell. In an embodiment, the constant region of one or preferably both chains is modified. For example, mutations may be made in the constant region of the extracellular region to promote pairing of the recombinant chains and reduce pairing with an endogenous TCR. One such example is the introduction into the constant region of the extracellular region of the α and β chains of a cysteine (C) residue such that the TCR forms a disulphide bridge not present in the naturally produced TCR. Suitably the C residue replaces a T, S or A residue, particularly a T or S residue. For example, position 164 of the extracellular region of the α chain within the constant region (naturally T—see SEQ ID NO: 9) is mutated to C (see SEQ ID NO: 11). For example, position 170 of the extracellular region of the β chain within the constant region (naturally S—see SEQ ID NO: 10) is mutated to C (see SEQ ID NO: 13). Preferably both of these changes are made.

Further useful mutations that may be made include mutations intended to increase the stability of a domain and/or promote correct folding, especially the extracellular domain of a soluble TCR. For example, residue 202 in the β chain (naturally N—see SEQ ID NO: 10) may be replaced with D (see SEQ ID NO: 12). Other possible mutations include the removal of native cysteine residues (particularly in the instance when new cysteine residues are introduced as mentioned above). Thus, existing C residues may be removed in the extracellular region (particularly within the constant region thereof) of either or both of the chains. For example, an existing C residue in the extracellular region of the β chain within the constant region may be replaced with A, S or T, especially A. An existing C residue in the extracellular region of the α chain within the constant region may also (or instead) be replaced with A, S or T, especially A. For example, position 188 of the extracellular region of the β chain within the constant region (naturally C—see SEQ ID NO: 10) may be mutated to A (see SEQ ID NO: 12). Position 187 of the extracellular region of the α chain within the constant region (naturally C—see SEQ ID NO: 9) may also (or instead) be mutated to A.

Sequences for expression in a bacterial host may be provided with an initial M residue.

### Sequence Fragments

Fragments of the TCR of the invention are provided which retain the function of cancer-specific binding.

Suitably, fragments substantially retain the ability of the TCR or fragment thereof to bind to its target epitope on the tumour, for example the binding affinity of the fragment is 75% or more e.g. 80% or more e.g. 85% or more e.g. 90% or more e.g. 95% or more e.g. 98% or more e.g. 99% or more of that of the TCR that in soluble form has α and β chains of respectively of any of (a) SEQ ID NOs: 9 and 10, (b) SEQ ID NOs: 53 and 54, (c) SEQ ID NOs: 55 and 56, (d) SEQ ID NOs: 57 and 58, or (e) SEQ ID NOs: 99 and 60.

In an embodiment, said TCR is a soluble TCR, or sTCR, and so lacks the transmembrane and, ideally also, intracellular domain. Thus, in an embodiment the TCR is a soluble form of a TCR which lacks the transmembrane and the intracellular domains i.e. it consists only of the extracellular domain.

In an embodiment, a single chain of the TCR is provided e.g. the α chain or the β chain. Thus, an example α chain sequence comprises or consists of SEQ ID NO: 9, 53, 55, 57, or 59 or a variant sequence having at least 85% sequence identity therewith. An example β chain sequence comprises or consists of SEQ ID NO: 10, 54, 56, 58, or 60 or a variant sequence having at least 85% sequence identity therewith.

A further exemplary fragment which is a fragment of an α chain comprises SEQ ID NOs: 1, 3 and 5, for example, comprises residues 27-106 of SEQ ID NO: 9, or comprises of SEQ ID Nos: (a) 33, 39, and 40, or (b) 34, 41, and 42. A further exemplary fragment which is a fragment of a β chain comprises SEQ ID NOs: 2, 4 and 6, for example, comprises residues 27-104 of SEQ ID NO: 10 or comprises of SEQ ID Nos: (a) 36, 5, and 6, or (b) 37, 5, and 6, or (c) 38, 43 and 44.

A further exemplary fragment comprises a fragment of an α chain which comprises SEQ ID NOs: 1, 3 and 5, for example, comprises residues 27-106 of SEQ ID NO: 9 or comprises of SEQ ID Nos: (a) 33, 39, and 40, or (b) 34, 41, and 42; and comprises a fragment of an β chain which comprises SEQ ID NOs: 2, 4 and 6, for example, comprises residues 27-104 of SEQ ID NO: 10 or comprises of SEQ ID Nos: (a) 36, 5, and 6, or (b) 37, 5, and 6, or (c) 38, 43 and 44.

A further exemplary fragment which is a fragment of an α chain comprises SEQ ID NO: 14, 45, 47, 49, or 51. A further exemplary fragment which is a fragment of a β chain comprises SEQ ID NO: 15, 46, 48, 50, or 52.

A further exemplary fragment comprises a fragment of an α chain and a fragment of an β chain respectively comprising (a) SEQ ID NO: 14 and SEQ ID NO: 15 (b) SEQ ID NO: 45 and SEQ ID NO: 46, (c) SEQ ID NO: 47 and SEQ ID NO: 48, (d) SEQ ID NO: 49 and SEQ ID NO: 50, or (e) SEQ ID NO: 52 and SEQ ID NO: 52.

In an embodiment, said TCR comprises an extracellular domain, a transmembrane domain and an intracellular domain. Hence each chain of the TCR comprises an extracellular region, a transmembrane region and an intracellular region.

Variants of fragments may also be contemplated (see above discussion of sequence variants).

### Polynucleotides and Vectors

According to a further aspect of the invention there is provided a polynucleotide encoding the TCR or cancer-specific binding fragment of the invention. A codon optimised coding sequence is derivable from the encoded sequence of the TCR or fragment thereof of the invention herein disclosed.

In an embodiment, a polynucleotide encoding the α chain of the TCR has or comprises the sequence corresponding to nucleotides 1-933 of SEQ ID NO: 16 (this being a codon optimised sequence).

In an embodiment, a polynucleotide encoding the β chain of the TCR has or comprises the sequence corresponding to nucleotides 1012-1839 of SEQ ID NO: 16 (this being a codon optimised sequence).

In an embodiment, such a polynucleotide may be a chimeric polynucleotide comprising a gene encoding the TCR or cancer-specific binding fragment and a heterologous promoter and/or other transcription control element such as a terminating signal operably linked thereto.

Since a complete TCR comprises an α chain and a β chain, two polynucleotides each encoding α chain of the TCR may be provided or a polynucleotide encoding both chains of the TCR may be provided. Yet further, the two chains of the TCR may be linked by a cleavable peptide linker (e.g. a linker that cleaves in an immune cell, particularly a T-cell; including a “self-cleaving” viral 2A sequence) and the polynucleotide may encode both chains of the TCR and the linker such as nucleic acids 934-1011 of SEQ ID NO: 16 (see SEQ ID NO: 16, discussed below).

The terms “nucleic acid” and “polynucleotide” are used interchangeably herein and refer to a polymeric macromolecule made from nucleotide monomers particularly deoxyribonucleotide or ribonucleotide monomers. The term encompasses polynucleotides containing known nucleotide analogues or modified backbone residues or linkages, which are naturally occurring and non-naturally occurring, which have similar properties as the reference polynucleotide, and which are intended to be metabolized in a manner similar to the reference nucleotides or are intended to have extended half-life in the system. Examples of such analogues include, without limitation, phosphorothioates, phosphoramidates, methyl phosphonates, chiral-methyl phosphonates, 2-O-methyl ribonucleotides, peptide-nucleic acids (PNAs). Suitably the term “polynucleotide” refers to naturally occurring polymers of deoxyribonucleotide or ribonucleotide monomers. Suitably the polynucleotides of the invention are recombinant. Recombinant means that the polynucleotide is the product of at least one of cloning, restriction or ligation steps, or other procedures that result in a polynucleotide that is distinct from a polynucleotide found in nature (e.g., in the case of cDNA). In an embodiment the polynucleotide of the invention is an artificial polynucleotide sequence (e.g., a cDNA sequence or polynucleotide sequence with non-naturally occurring codon usage). In one embodiment, the polynucleotides of the invention are DNA. Alternatively, the polynucleotides of the invention are RNA.

DNA (deoxyribonucleic acid) and RNA (ribonucleic acid) refer to polynucleotides having a backbone of sugar moieties which are deoxyribosyl and ribosyl moieties respectively. The sugar moieties may be linked to bases which are the 4 natural bases (adenine (A), guanine (G), cytosine (C) and thymine (T) in DNA and adenine (A), guanine (G), cytosine (C) and uracil (U) in RNA). As used herein, a “corresponding RNA” is an RNA having the same sequence as a reference DNA but for the substitution of thymine (T) in the DNA with uracil (U) in the RNA. The sugar moieties may also be linked to unnatural bases such as inosine, xanthosine, 7-methylguanosine, dihydrouridine and 5-methylcytidine. Natural phosphodiester linkages between sugar (deoxyribosyl/ribosyl) moieties may optionally be replaced with phosphorothioates linkages. Suitably polynucleotides of the invention consist of the natural bases attached to a deoxyribosyl or ribosyl sugar backbone with phosphodiester linkages between the sugar moieties.

In an embodiment the polynucleotide of the invention is a DNA, including single- or double-stranded DNA and straight-chain or circular DNA (i.e. plasmid DNA).

Due to the degeneracy of the genetic code, a large number of different, but functionally identical polynucleotides can encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such polynucleotide variations lead to “silent” (sometimes referred to as “degenerate” or “synonymous”) variants, which are one species of conservatively modified variations. Every polynucleotide sequence disclosed herein which encodes a polypeptide also enables every possible silent variation of the polynucleotide. One of skill will recognise that each codon in a polynucleotide (except AUG, which is ordinarily the only codon for methionine, and UGG, which is ordinarily the only codon for tryptophan) can be modified to yield a functionally identical molecule. Accordingly, each silent variation of a polynucleotide that encodes a polypeptide is implicit in each described sequence and is provided as an aspect of the invention.

Degenerate codon substitutions may also be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; Rossolini et al., 1994, Mol. Cell. Probes 8:91-98).

Codons of the polynucleotide sequences of the invention may be altered in order that sequence variants of the TCR are expressed as discussed above. In an embodiment, up to 5 codons are altered e.g. one, two or three e.g. one or two e.g. one codons are altered such that a different amino acid is encoded where the codon alteration occurs. Codon alterations may involve the alteration of one, two or three bases in the polynucleotide according to the amino acid alteration to be achieved. Suitably, codons encoding residues of the CDRs are not altered.

In an embodiment, the polynucleotides of the invention are codon optimised for expression in a human host cell, particularly, an immune cell, particularly a T-cell.

According to a yet further aspect of the invention there is provided a vector encoding said TCR or cancer-specific binding fragment of the invention. Specifically, there is provided a vector for delivery of a polynucleotide to cells (particularly immune cells, such as T-cells) comprising a polynucleotide encoding the TCR or cancer-specific binding fragment of the invention.

As noted above, since a complete TCR comprises an α chain and a β chain, two vectors each comprising a polynucleotide encoding α chain of the TCR may be provided or a vector comprising a polynucleotide encoding both chains of the TCR may be provided. Yet further, the two chains of the TCR may be linked by a cleavable peptide linker (e.g. a linker that cleaves in an immune cell, particularly a T-cell) and the vector may comprise a polynucleotide which encodes both chains of the TCR and the linker.

The, or each vector should suitably comprise such elements as are necessary for permitting transcription of a translationally active RNA molecule in the host cell, such as a promoter and/or other transcription control elements such as an internal ribosome entry site (IRES) or a termination signal. A “translationally active RNA molecule” is an RNA molecule capable of being translated into a protein by the host cell's translation apparatus.

Example promoters to drive transcription of the TCR chains include constitutive promoters such as the cytomegalovirus (CMV) promoter and elongation factor 1α (EF1α) promoter.

The vector may be, for example, a viral vector such as a lentiviral vector. Other examples of viral vectors include vectors derived from γ-retrovirus, adenovirus, adeno-associated virus (AAV), alphavirus, herpes virus, arenavirus, measles virus, poxvirus or rhabdovirus. DNA molecules, for example transposons, may also be suitable vectors to transduce immune cells, such as T-cells with TCR genes.

A suitable polynucleotide of the invention may encode a TCR or cancer-specific binding fragment of a TCR according to the invention, wherein the TCR or binding fragment comprises:
(a) 3 α-chain CDRs comprising or consisting of CAVRLAGYGGSQGNLIF, (SEQ ID NO: 1) or a variant CDR that has 1, 2, 3, 4 or 5 amino acid variations with respect thereto; DSAIYN, (SEQ ID NO: 3) and IQSSQREQ, (SEQ ID NO: 4) or a variant CDR that has 1 or 2 amino acid variations with respect thereto, such as 1 variation; and comprises 3 β-chain CDRs comprising or consisting of CASSSQGTDTQYF, (SEQ ID NO: 2) or a variant CDR that has 1, 2, 3, 4 or 5 amino acid variations with respect thereto, and SGHDY, (SEQ ID NO: 5) and FNNNVP, (SEQ ID NO: 6) or a variant CDR that has 1 or 2 amino acid variations with respect thereto, such as 1 variation, or
(b) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRGNTGELFF (SEQ ID NO: 36), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation; or
(c) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRTGQGNQPQHF (SEQ ID NO: 37), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation; or
(d) 3 α-chain CDRs comprising or consisting of CAVREADYGGSQGNLIF (SEQ ID NO: 34) or a variant CDR that has 1, 2, 3, 4 or 5 amino acid variations with respect thereto; and VGISA, (SEQ ID NO: 41) and LSSGK, (SEQ ID NO: 42) or a variant CDR that has 1 or 2 amino acid variations with respect thereto, such as 1 variation; and comprises 3-β chain CDRs comprising or consisting of CASSLEQGQYF (SEQ ID NO: 38), or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; or
(e) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation and comprises 3-β chain CDRs comprising or consisting of CASSLEQGQYF (SEQ ID NO: 38) or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; or
(f) an α chain extracellular region comprising or consisting of SEQ ID NO: 11 or a variant α chain extracellular region which has at least 85% sequence identity to the α chain extracellular region of SEQ ID NO: 11 and a β chain extracellular region comprising SEQ ID NO: 12 or a variant β chain extracellular region which has at least 85% sequence identity to the β chain extracellular region of SEQ ID NO: 12 or a β chain extracellular region comprising SEQ ID NO: 13 or a variant β chain extracellular region which has at least 85% sequence identity to the β chain extracellular region of SEQ ID NO: 13; or
(g) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising or consisting of SEQ ID NO: 10 or a variant β chain extracellular region which has at least 85% sequence identity to the β chain extracellular region of SEQ ID NO: 10; or
(h) an α chain comprising or consisting of SEQ ID NO: 29 or a variant α chain which has at least 85% sequence identity to the α chain of SEQ ID NO: 29 and a β chain comprising or consisting of SEQ ID NO: 31 or a variant β chain which has at least 85% sequence identity to the β chain of SEQ ID NO: 31; or
(i) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising SEQ ID NO: 12 or a variant β chain extracellular region which has at least 85% sequence identity to the @ chain extracellular region of SEQ ID NO: 12 or a β chain extracellular region comprising SEQ ID NO: 13 or a variant β chain extracellular region which has at least 85% sequence identity to the β chain extracellular region of SEQ ID NO: 13; or
(j) an α chain variable region comprising or consisting of SEQ ID NO: 14 or a variant α chain variable region which has at least 85% sequence identity to the α chain variable region of SEQ ID NO: 14 and a β chain variable region comprising or consisting of SEQ ID NO: 15 or a variant β chain variable region which has at least 85% sequence identity to the β chain variable region of SEQ ID NO: 15; or
(k) an α chain variable region comprising or consisting of SEQ ID NO: 45 or a variant α chain variable region which has at least 85% sequence identity to the α chain variable region of SEQ ID NO: 45 and a β chain variable region comprising or consisting of SEQ ID NO: 46 or a variant β chain variable region which has at least 85% sequence identity to the β chain variable region of SEQ ID NO: 46; or
(l) an α chain variable region comprising or consisting of SEQ ID NO: 47 or a variant α chain variable region which has at least 85% sequence identity to the α chain variable region of SEQ ID NO: 47 and a β chain variable region comprising or consisting of SEQ ID NO: 48 or a variant β chain variable region which has at least 85% sequence identity to the β chain variable region of SEQ ID NO: 48; or
(n) an α chain variable region comprising or consisting of SEQ ID NO: 49 or a variant α chain variable region which has at least 85% sequence identity to the α chain variable region of SEQ ID NO: 49 and a β chain variable region comprising or consisting of SEQ ID NO: 50 or a variant β chain variable region which has at least 85% sequence identity to the β chain variable region of SEQ ID NO: 50; or
(m) an α chain variable region comprising or consisting of SEQ ID NO: 51 or a variant α chain variable region which has at least 85% sequence identity to the α chain variable region of SEQ ID NO: 51 and a β chain variable region comprising or consisting of SEQ ID NO: 52 or a variant β chain variable region which has at least 85% sequence identity to the β chain variable region of SEQ ID NO: 52;
optionally wherein in each case the variations are selected from additions, substitutions and deletions and wherein the at least 85% identity may be any one of 85, 86, 87, 88, 89, 90, 91, 92, 93, 94, 95, 96, 97, 98, 99 or 100% identity.

In on embodiment said polynucleotide may suitably encode an immature TCR which comprises an N-terminal leader sequence (residues 1-19 of SEQ ID NO: 7 and residues 1-19 of SEQ ID NO: 8) which N-terminal leader sequence is removed by cellular peptidases (such as signal peptidase) to produce the mature form.

A suitable vector of the invention comprises the aforementioned polynucleotide and is, for example, a viral vector as disclosed above such as a lentiviral vector.

### Production of TCRs

TCRs of the invention and the derivatives described herein (bispecifics etc) can be obtained and manipulated using the techniques disclosed for example in Green and Sambrook 2012 Molecular Cloning: A Laboratory Manual 4th Edition Cold Spring Harbour Laboratory Press. In particular, artificial gene synthesis may be used to produce polynucleotides (Nambiar et al., 1984, Science, 223:1299-1301, Sakamar and Khorana, 1988, Nucl. Acids Res., 14:6361-6372, Wells et al., 1985, Gene, 34:315-323 and Grundstrom et al., 1985, Nucl. Acids Res., 13:3305-3316) followed by expression in a suitable organism to produce polypeptides. A gene encoding a polypeptide of the invention can be synthetically produced by, for example, solid-phase DNA synthesis. Entire genes may be synthesized de novo, without the need for precursor template DNA. To obtain the desired oligonucleotide, the building blocks are sequentially coupled to the growing oligonucleotide chain in the order required by the sequence of the product. Upon the completion of the chain assembly, the product is released from the solid phase to solution, deprotected, and collected. Products can be isolated by high-performance liquid chromatography (HPLC) to obtain the desired oligonucleotides in high purity (Verma and Eckstein, 1998, Annu. Rev. Biochem. 67:99-134). These relatively short segments are readily assembled by using a variety of gene amplification methods (Methods Mol Biol., 2012; 834:93-109) into longer DNA molecules, suitable for use in innumerable recombinant DNA-based expression systems. In the context of this invention one skilled in the art would understand that the polynucleotide sequences encoding the TCRs and fragments thereof described in this invention could be readily used in a variety of protein production systems, including, for example, viral vectors.

For the purposes of production of polypeptides of the invention in a microbiological host (e.g., bacterial or fungal), polynucleotides of the invention will comprise suitable regulatory and control sequences (including promoters, termination signals etc) and sequences to promote polypeptide secretion suitable for protein production in the host.

Similarly, polypeptides of the invention could be produced by transducing cultures of eukaryotic cells (e.g., Chinese hamster ovary cells or drosophila S2 cells) with polynucleotides of the invention which have been combined with suitable regulatory and control sequences (including promoters, termination signals etc) and sequences to promote polypeptide secretion suitable for protein production in these cells.

Improved isolation of the polypeptides of the invention produced by recombinant means may optionally be facilitated through the addition of a purification tag at one end of the polypeptide. An example purification tag is a stretch of histidine residues (e.g. 6-10 His residues), commonly known as a His-tag. Other example purification tags include a MYC tag with amino acid sequence EQKLISEEDL (SEQ ID NO: 20), a FLAG tag with sequence DYKDDDDK (SEQ ID NO: 21) or an HA tag with sequence YPYDVPDYA (SEQ ID NO: 22).

The polypeptides of the invention may be produced ex vivo in immune cells, such as T-cells as discussed below.

### Immune Cells and Immune Cell Clones

The immune cells and immune cell clones which may express the TCR of the invention or into which the TCR may be transduced include cytotoxic cells such as T-cells, NK-cells and NKT-cells, particularly T-cells. The immune cells and immune cell clones may be cells of the lymphoid lineage including T cells, Natural Killer T (NKT) cells, and precursors thereof including embryonic stem cells, and pluripotent stem cells from which lymphoid cells may be differentiated. Preferably the immune cells and immune cell clones are T cells which can for example include, but are not limited to, helper T cells, cytotoxic T cells, memory T cells (including central memory T cells, stem-cell-like memory T cells or stem-like memory T cells), and effector memory T cells such as TEM cells and TEMRA cells, Regulatory T cells or suppressor T cells, Natural killer T cells, Mucosal associated invariant T cells, TILs (tumour infiltrating lymphocytes) and gamma-delta T cells. Preferably, the immune cells and immune cell clones are T cells optionally CD4⁺ T cells or a CD8⁺ T cells. Accordingly the immune cells and immune cell clones may be T-cells, optionally CD4+ T cells or CD8+ T cells, or the immune cells and immune cell clones may be a population of T-cells, optionally CD4+ T cells; or CD8+ T cells, or a mixed population of CD4+ T cells and CD8+ T cells.

According to a further aspect of the invention there is provided an immune cell, particularly a T-cell expressing said TCR or cancer-specific binding fragment of the invention, ideally, in either a soluble or membrane compatible form i.e. having a transmembrane region and intracellular region.

According to a yet further aspect of the invention there is provided an immune cell clone, particularly a T-cell clone expressing said TCR or cancer-specific binding fragment of the invention, ideally, in either a soluble or membrane-compatible form i.e. having a transmembrane region and intracellular region. Preferably said clone is a MC.27.759S, A3, A4, C1 or C2 clone as described herein.

According to an aspect of the invention there is provided an engineered immune cell or immune cell clone, particularly a T-cell or T-cell clone which expresses a TCR of the invention, in particular, it expresses a TCR which comprises:
a TCR or cancer-specific binding fragment of a TCR according to the invention, wherein the TCR or binding fragment comprises:
(a) 3 α-chain CDRs comprising or consisting of CAVRLAGYGGSQGNLIF, (SEQ ID NO: 1) or a variant CDR that has 1, 2, 3, 4 or 5 amino acid variations with respect thereto; DSAIYN, (SEQ ID NO: 3) and IQSSQREQ, (SEQ ID NO: 4) or a variant CDR that has 1 or 2 amino acid variations with respect thereto, such as 1 variation; and comprises 3 β-chain CDRs comprising or consisting of CASSSQGTDTQYF, (SEQ ID NO: 2) or a variant CDR that has 1, 2, 3, 4 or 5 amino acid variations with respect thereto, and SGHDY, (SEQ ID NO: 5) and FNNNVP, (SEQ ID NO: 6) or a variant CDR that has 1 or 2 amino acid variations with respect thereto, such as 1 variation, or
(b) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRGNTGELFF (SEQ ID NO: 36), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation; or
(c) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRTGQGNQPQHF (SEQ ID NO: 37), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation; or
(d) 3 α-chain CDRs comprising or consisting of CAVREADYGGSQGNLIF (SEQ ID NO: 34) or a variant CDR that has 1, 2, 3, 4 or 5 amino acid variations with respect thereto; and VGISA, (SEQ ID NO: 41) and LSSGK, (SEQ ID NO: 42) or a variant CDR that has 1 or 2 amino acid variations with respect thereto, such as 1 variation; and comprises 3-β chain CDRs comprising or consisting of CASSLEQGQYF (SEQ ID NO: 38), or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; or
(e) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation and comprises 3-β chain CDRs comprising or consisting of CASSLEQGQYF (SEQ ID NO: 38) or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; or
(f) an α chain extracellular region comprising or consisting of SEQ ID NO: 11 or a variant α chain extracellular region which has at least 85% sequence identity to the α chain extracellular region of SEQ ID NO: 11 and a β chain extracellular region comprising SEQ ID NO: 12 or a variant β chain extracellular region which has at least 85% sequence identity to the β chain extracellular region of SEQ ID NO: 12 or a β chain extracellular region comprising SEQ ID NO: 13 or a variant β chain extracellular region which has at least 85% sequence identity to the β chain extracellular region of SEQ ID NO: 13; or
(g) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising or consisting of SEQ ID NO: 10 or a variant β chain extracellular region which has at least 85% sequence identity to the β chain extracellular region of SEQ ID NO: 10; or
(h) an α chain comprising or consisting of SEQ ID NO: 29 or a variant α chain which has at least 85% sequence identity to the α chain of SEQ ID NO: 29 and a β chain comprising or consisting of SEQ ID NO: 31 or a variant β chain which has at least 85% sequence identity to the β chain of SEQ ID NO: 31; or
(i) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising SEQ ID NO: 12 or a variant β chain extracellular region which has at least 85% sequence identity to the β chain extracellular region of SEQ ID NO: 12 or a β chain extracellular region comprising SEQ ID NO: 13 or a variant β chain extracellular region which has at least 85% sequence identity to the β chain extracellular region of SEQ ID NO: 13; or
(j) an α chain variable region comprising or consisting of SEQ ID NO: 14 or a variant α chain variable region which has at least 85% sequence identity to the α chain variable region of SEQ ID NO: 14 and a β chain variable region comprising or consisting of SEQ ID NO: 15 or a variant β chain variable region which has at least 85% sequence identity to the β chain variable region of SEQ ID NO: 15; or
(k) an α chain variable region comprising or consisting of SEQ ID NO: 45 or a variant α chain variable region which has at least 85% sequence identity to the α chain variable region of SEQ ID NO: 45 and a β chain variable region comprising or consisting of SEQ ID NO: 46 or a variant β chain variable region which has at least 85% sequence identity to the β chain variable region of SEQ ID NO: 46; or
(l) an α chain variable region comprising or consisting of SEQ ID NO: 47 or a variant α chain variable region which has at least 85% sequence identity to the α chain variable region of SEQ ID NO: 47 and a β chain variable region comprising or consisting of SEQ ID NO: 48 or a variant β chain variable region which has at least 85% sequence identity to the β chain variable region of SEQ ID NO: 48; or
(n) an α chain variable region comprising or consisting of SEQ ID NO: 49 or a variant α chain variable region which has at least 85% sequence identity to the α chain variable region of SEQ ID NO: 49 and a β chain variable region comprising or consisting of SEQ ID NO: 50 or a variant β chain variable region which has at least 85% sequence identity to the β chain variable region of SEQ ID NO: 50; or
(m) an α chain variable region comprising or consisting of SEQ ID NO: 51 or a variant α chain variable region which has at least 85% sequence identity to the α chain variable region of SEQ ID NO: 51 and a β chain variable region comprising or consisting of SEQ ID NO: 52 or a variant β chain variable region which has at least 85% sequence identity to the β chain variable region of SEQ ID NO: 52;
optionally wherein in each case the variations are selected from additions, substitutions and deletions and wherein the at least 85% identity may be any one of 85, 86, 87, 88, 89, 90, 91, 92, 93, 94, 95, 96, 97, 98, 99 or 100% identity.

Preferably the T cell is a CD4⁺ T cell or a CD8⁺ T cell. The T cell may be a population of T-cells, optionally CD4+ T cells; or CD8+ T cells, or a mixed population of CD4+ T cells and CD8+ T cells. Suitably the T-cell is a CD8+ T-cell.

In an embodiment the immune cell or immune cell clone, particularly the T-cell or T-cell clone is engineered. For example, the invention provides a immune cell or immune cell clone, particularly a T-cell or T-cell clone wherein the cell or cells of the clone are modified by the introduction (e.g. by transduction) of a heterologous polynucleotide encoding the TCR or cancer-specific binding fragment of the invention or by a vector of the invention.

In an embodiment, the immune cell, particularly the T-cell comprises a vector comprising a polynucleotide encoding the TCR of the invention and the immune cell, particularly the T-cell expresses the TCR of the invention.

According to an aspect of the invention there is provided an ex vivo process comprising (i) obtaining immune cells, particularly T-cells from a patient, (ii) introducing (e.g. by transduction) a heterologous polynucleotide of the invention or a vector of the invention into the immune cells, particularly T-cells to produce modified immune cells, particularly T-cells which express a cancer-specific T-cell receptor (TCR) or a cancer-specific binding fragment of a TCR according to the invention; and (iii) reintroducing said modified immune cells, particularly T-cells into the patient. There is also provided a process comprising reintroducing modified immune cells, particularly T-cells into a patient wherein said modified immune cells, particularly T-cells are obtained from said patient and modified ex vivo (e.g. by transduction) by the introduction of a heterologous polynucleotide of the invention or by a vector of the invention so that they express a cancer-specific T-cell receptor (TCR) or a cancer-specific binding fragment of a TCR according to the invention. The patient in question suitably is a cancer patient particularly a human cancer patient.

The immune cells, particularly T-cells may optionally be expanded before or (more preferably) after step (ii) above. The immune cells, particularly T-cells may be expanded by multiple methods, e.g. by treatment with a cytokine and/or stimulatory antibody for example IL-2 and/or antibodies against CD3 and CD28.

In a further embodiment which is a variant of the aforementioned processes, the transduced immune cells, particularly T-cells which are administered to the patients were not originally from the same patient (i.e. they are allogeneic cells).

The immune cells, particularly T-cells that are introduced into the patient after modification (e.g. by transduction) may be polyclonal or monoclonal. In the latter case, a particular modified (e.g. transduced) clone is selected for expansion before administering the cells to the patient.

There is also provided a method of treatment of cancer comprising administering to a patient in need thereof modified immune cells, particularly T-cells wherein the immune cells, particularly T-cells are immune cells, particularly T-cells which have been obtained from said patient and modified ex vivo (e.g. by transduction) by the introduction of a heterologous polynucleotide of the invention or by a vector of the invention so that they express a cancer-specific T-cell receptor (TCR) or a cancer-specific binding fragment of a TCR according to the invention.

There are also provided modified immune cells, particularly T-cells for use in the treatment of cancer wherein the immune cells, particularly T-cells are immune cells, particularly T-cells which have been obtained from said patient and modified (e.g. by transduction) ex vivo by the introduction of a heterologous polynucleotide of the invention or by a vector of the invention so that they express a cancer-specific T-cell receptor (TCR) or a cancer-specific binding fragment of a TCR according to the invention.

There is also provided use of the aforementioned modified (e.g. transduced) immune cells, particularly T-cells in the manufacture of a medicament for the treatment of cancer.

As noted above, since a complete TCR comprises an α chain and a β chain, the immune cells, particularly T-cells may (i) be modified by the introduction (e.g. by transduction) of two polynucleotides each encoding a chain of the TCR or with two vectors each comprising a polynucleotide encoding a chain of the TCR or (ii) may be modified by the introduction (e.g. by transduction) of a polynucleotide (e.g. a transposon) encoding both chains of the TCR or a vector comprising a polynucleotide encoding both chains of the TCR. Yet further, the two chains of the TCR may be linked by a cleavable peptide linker (e.g. a linker that cleaves in an immune cell, particularly a T-cell) and so the immune cells, particularly T-cells may be modified by the introduction (e.g. by transduction) of a polynucleotide which encodes both chains of the TCR and the linker or with a vector comprising a polynucleotide which encodes both chains of the TCR and the linker such that the two chains of the TCR are produced in the immune cell, particularly the T-cell.

### Fusion Proteins

As discussed above, it may be convenient to produce the TCR as a fusion protein comprising the α and β chains (or fragments thereof comprising at least one CDR, preferably 3 CDRs per chain) which is cleaved to form the respective α and β chains in a cell (e.g. an immune cell, particularly a T-cell). Hence the two chains or fragments thereof are connected by a cleavable linker.

Suitable cleavable linkers include self-cleaving linkers of the 2A family, which can include

P2A
(ATNFSLLKQAGDVEENPGP, SEQ ID NO: 24),
E2A
(QCTNYALLKLAGDVESNPGP, SEQ ID NO: 25),
F2A
(VKQTLNFDLLKLAGDVESNPGP, SEQ ID NO: 26)
and
T2A
(EGRGSLLTCGDVEENPGP, SEQ ID NO: 27).

P2A is derived from porcine teschovirus-1 2A, E2A is derived from equine rhinitis A virus, F2A is derived from foot-and-mouth disease virus, T2A is derived from thosea asigna virus 2A. The linker of the 2A family may be preceded by a furin cleavage site and a SGSG (SEQ ID NO: 28) linker sequence. See Example 8 for an example embodiment. The sequence of an exemplary polynucleotide that encodes a fusion protein comprising a TCR α chain (SEQ ID NO: 7) and a TCR β chain (SEQ ID NO: 8) connected via a self-cleaving P2A linker preceded by a furin cleavage site and SGSG (SEQ ID NO: 28) linker (nucleotide sequence indicated by bold underlining) and which has been codon optimised is given below:

(SEQ ID NO: 16)
ATGGGCAGCTGGACCCTGTGTTGTGTGTCCCTGTGTATCCTGGTGGCCA
AGCACACAGATGCCGGCGTGATCCAGTCTCCTAGACACGAAGTGACCGA
GATGGGCCAAGAAGTGACCCTGCGCTGCAAGCCTATCAGCGGCCACGAT
TACCTGTTCTGGTACAGACAGACCATGATGAGAGGCCTGGAACTGCTGA
TCTACTTCAACAACAACGTGCCCATCGACGACAGCGGCATGCCCGAGGA
TAGATTCAGCGCCAAGATGCCCAACGCCAGCTTCAGCACCCTGAAGATC
CAGCCTAGCGAGCCCAGAGATAGCGCCGTGTACTTTTGTGCCAGCAGCA
GCCAGGGCACCGACACACAGTATTTTGGCCCTGGCACCAGACTGACCGT
GCTGGAAGATCTGAAGAACGTGTTCCCTCCAAAGGTGGCCGTGTTCGAG
CCTTCTGAGGCCGAGATCAGCCACACACAGAAAGCCACACTCGTGTGTC
TGGCCACCGGCTTCTATCCCGATCACGTGGAACTGTCTTGGTGGGTCAA
CGGCAAAGAGGTGCACAGCGGCGTCAGCACAGATCCCCAGCCTCTGAAA
GAACAGCCCGCTCTGAACGACAGCCGGTACTGTCTGAGCAGCAGACTGA
GAGTGTCCGCCACCTTCTGGCAGAACCCCAGAAACCACTTCAGATGCCA
GGTGCAGTTCTACGGCCTGAGCGAGAACGATGAGTGGACCCAGGATAGA
GCCAAGCCTGTGACACAGATCGTGTCTGCCGAAGCCTGGGGCAGAGCCG
ATTGTGGCTTTACCAGCGAGAGCTACCAGCAGGGCGTGCTGTCTGCCAC
AATCCTGTACGAGATCCTGCTGGGCAAAGCCACTCTGTACGCCGTGCTG
GTGTCTGCTCTGGTGCTGATGGCCATGGTCAAGCGGAAGGACTCTAGAG
GCAGAGCCAAGAGAAGCGGCTCTGGCGAAGGCAGAGGTAGCCTGCTTAC
ATGCGGCGACGTGGAAGAGAACCCCGGACCTATGGAAACACTGCTGGGC
CTGCTGATCCTGTGGCTGCAACTGCAATGGGTGTCCAGCAAGCAAGAAG
TCACCCAGATTCCAGCCGCTCTGTCTGTGCCTGAGGGCGAAAACCTGGT
CCTGAACTGCAGCTTCACCGACAGCGCCATCTACAACCTGCAGTGGTTC
AGACAGGACCCCGGCAAGGGACTGACAAGCCTGCTGCTGATTCAGAGCA
GCCAGAGAGAGCAGACCAGCGGCAGACTGAATGCCAGCCTGGATAAGTC
CTCCGGCAGAAGCACCCTGTATATCGCCGCATCTCAGCCTGGCGATAGC
GCCACATATCTGTGCGCCGTTAGACTGGCCGGCTATGGCGGATCTCAGG
GCAATCTGATCTTCGGCAAGGGCACCAAGCTGAGCGTGAAGCCCAACAT
TCAGAACCCCGATCCTGCCGTGTACCAGCTGAGAGACAGCAAGAGCAGC
GACAAGAGCGTGTGCCTGTTCACCGACTTCGACAGCCAGACCAACGTGT
CCCAGAGCAAGGACAGCGACGTGTACATCACCGACAAGACAGTGCTGGA
CATGCGGAGCATGGACTTCAAGAGCAACAGCGCCGTGGCCTGGTCCAAC
AAGAGCGATTTCGCCTGCGCCAACGCCTTCAACAATAGCATTATCCCCG
AGGACACATTCTTCCCAAGTCCTGAGAGCAGCTGCGACGTGAAGCTGGT
GGAAAAGAGCTTCGAGACAGACACCAACCTGAACTTCCAGAACCTGTCC
GTGATCGGCTTCCGGATCCTGCTCCTGAAAGTGGCCGGCTTCAACCTGC
TGATGACCCTGAGACTGTGGTCCAGC.,

In SEQ ID NO: 16, the α chain of the TCR is encoded by nucleotides 1-933, linker is nucleic acids 934-1011 and the β chain of the TCR is encoded by nucleotides 1012-1839.

The sequence of SEQ ID NO: 16 does not include a stop codon. A suitable stop codon e.g. TAA will typically be included at the C terminus in a construct including this sequence.

The fusion protein sequence encoded by the sequence of SEQ ID NO: 16 is given below as SEQ ID NO: 30:

(SEQ ID NO: 30)
MGSWTLCCVSLCILVAKHTDAGVIQSPRHEVTEMGQEVTLRCKPISGHD
YLFWYRQTMMRGLELLIYFNNNVPIDDSGMPEDRFSAKMPNASFSTLKI
QPSEPRDSAVYFCASSSQGTDTQYFGPGTRLTVLEDLKNVFPPKVAVFE
PSEAEISHTQKATLVCLATGFYPDHVELSWWVNGKEVHSGVSTDPQPLK
EQPALNDSRYCLSSRLRVSATFWQNPRNHFRCQVQFYGLSENDEWTQDR
AKPVTQIVSAEAWGRADCGFTSESYQQGVLSATILYEILLGKATLYAVL
VSALVLMAMVKRKDSRGRAKRSGSGEGRGSLLTCGDVEENPGPMETLLG
LLILWLQLQWVSSKQEVTQIPAALSVPEGENLVLNCSFTDSAIYNLQWF
RQDPGKGLTSLLLIQSSQREQTSGRLNASLDKSSGRSTLYIAASQPGDS
ATYLCAVRLAGYGGSQGNLIFGKGTKLSVKPNIQNPDPAVYQLRDSKSS
DKSVCLFTDFDSQTNVSQSKDSDVYITDKTVLDMRSMDFKSNSAVAWSN
KSDFACANAFNNSIIPEDTFFPSPESSCDVKLVEKSFETDTNLNFQNLS
VIGFRILLLKVAGFNLLMTLRLWSS.

### Multimers

The TCR of the invention, particularly in its soluble form, may be employed in multimeric form, for example a tetramer, pentamer. The multimer may also be a dextramer. Such multimers may be used for identifying and/or capturing cells that express the target MR1-ligand complex on the surface. For example, the TCR of the invention may be provided with a biotin tag and complexed in multimeric form to streptavidin. Streptavidin may be complexed to a detection signal e.g. a fluorophore.

### Bispecifics

According to a yet further aspect of the invention there is provided a pharmaceutical composition or bispecific comprising said TCR or cancer-specific binding fragment thereof or cell or clone or vector.

Alternatively still, said TCR (or cancer-specific binding fragments thereof) may form part of a bispecific wherein said bispecific includes said TCR, for the purpose of binding to its ligand on a cancer cell, and also an immune cell activating component or ligand that binds and so activates an immune cell such as a Killer T-cell.

In an embodiment, there is provided a bispecific construct comprising the TCR or cancer-specific binding fragment of the invention (such as a soluble TCR) and an immune cell activating component or ligand (such as an antibody protein) that binds to and activates an immune cell, for example such as T-cell e.g. a CD8+ T-cell. For example, the immune cell activating component or ligand activates an immune cell via binding to CD3.

For example, one particular bispecific construct comprises the polypeptide sequences of the TCR or cancer-specific binding fragment of the invention, for example a soluble TCR of the invention, and an antibody protein (particularly an agonist antibody protein) that binds to CD3.

Said antibody protein is suitably an antigen-binding domain of an antibody such as a VH (variable heavy domain from a 4 chain antibody) or a VHH (variable heavy domain from a 2 chain (heavy chain only) antibody such as from Camelid) or is a scFv (i.e. a fusion protein comprising the variable regions of the light and heavy chains of an antibody connected by a short (e.g. 10-25 amino acids) linker).

A number of anti-CD3 agonist antibodies are available in the prior art. For example, blinatumomab which is an approved bispecific T-cell engager (BiTE) product consists of a CD19-targeting antibody (heavy chain scFv) connected to a CD3-targeting agonist antibody (light chain scFv). The CD3-targeting agonist antibody (light chain scFv) component of blinatumomab could therefore be used. The other BiTE products solitomab, pasotuxizumab and ertumaxomab also comprise a CD3-targeting agonist antibody component which could be used in a bispecific of the present invention. The TCR-based bispecific tebentafusp also comprises a CD3-targeting agonist antibody component which could be used in a bispecific of the present invention.

A linker may be provided to link α chain of the TCR to the other part of the bispecific construct e.g. a non-immunogenic linker sequence such as GGGGS (SEQ ID NO: 17).

More generally, there is provided a fusion protein comprising the TCR or cancer-specific binding fragment of the invention and a heterologous protein providing the immune cell stimulating and/or activating activity, for example an antibody, which may be a monoclonal antibody, or antibody fragment or binding domain, for example any of Fab, Fab′, diabody, tribody, minibody, scFv, scFv-Fc, F(ab′)2, VhH, V-NAR, dab, dab-Fc, free-LC, half antibody.

There is also provided a polynucleotide encoding said bispecific, bispecific construct or fusion protein. Said polynucleotide is suitably a DNA, including single or double stranded DNA and straight-chain or circular DNA.

### Compositions

The invention provides a pharmaceutical composition comprising the TCR or cancer-specific binding fragment thereof, polynucleotide, vector, immune cell, immune cell clone, T-cell, T-cell clone, bispecific, bispecific construct or fusion protein of the invention and a pharmaceutically acceptable carrier. Suitably the pharmaceutical composition is formulated under sterile conditions and is suitable for parenteral administration. For parenteral administration, the carrier preferably comprises water and may contain buffers for pH control, stabilising agents e.g., surfactants and amino acids and tonicity modifying agents e.g., salts and sugars.

### Cancer Treatment

According to a further aspect of the invention there is provided a method of treating cancer comprising administering said TCR (or cancer-specific binding fragment thereof) or cell or clone or polynucleotide or vector of the invention to an individual to be treated.

There is also provided said TCR (or cancer-specific binding fragment thereof) or cell or clone, for example immune cell, immune cell clone, T-cell, T-cell clone or polynucleotide or vector of the invention for use in the treatment of cancer.

There is also provided the use of said TCR or cell or clone, for example immune cell, immune cell clone, T-cell, T-cell clone, or vector of the invention in the manufacture of a medicament to treat cancer.

Also, there is provided a method of treating cancer in a subject comprising administering a therapeutically effective amount of an immune cell, immune cell clone, T-cell, T-cell clone, bispecific, bispecific construct, fusion protein or pharmaceutical composition of the invention to the subject.

There is also provided an immune cell, immune cell clone, T-cell, T-cell clone, bispecific, bispecific construct, fusion protein or pharmaceutical composition of the invention for use in the treatment of cancer.

There is also provided use of an immune cell, immune cell clone, T-cell, T-cell clone, bispecific, bispecific construct, fusion protein or pharmaceutical composition of the invention in the manufacture of a medicament to treat cancer.

Said cancer may include solid tumours and blood cancers, for example cancers selected from lung, melanoma (e.g. skin melanoma), bone, breast, blood (e.g. leukemia), prostate, kidney, bladder, cervical, ovarian and colorectal cancers, and in particular selected from lung, melanoma (e.g. skin melanoma), bone and breast cancers.

Equally well, it is also envisaged that all embodiments of the present invention that may be used to treat cancer may potentially be used to prevent cancer. Thus, corresponding methods and uses and substances for use to prevent cancer are provided as an aspect of the invention.

### Combinations

According to a yet further aspect of the invention there is provided a combination therapeutic for the treatment of cancer comprising:
a) said TCR (or cancer-specific binding fragment thereof) or cell or clone, for example immune cell, immune cell clone, T-cell, T-cell clone, or polynucleotide or vector of the invention in combination with
b) a further cancer therapeutic agent.
Accordingly, the TCR (or cancer-specific binding fragment thereof) or cell or clone or polynucleotide or vector of the invention may be administered separately, simultaneously or sequentially with an anti-cancer agent.

More generally, there is provided a pharmaceutical composition comprising:
a) the immune cell, immune cell clone, T-cell, T-cell clone, pharmaceutical composition, bispecific, bispecific construct or fusion protein of the invention; and
b) an anti-cancer agent.

Accordingly, the immune cell, immune cell clone, T-cell, T-cell clone, pharmaceutical composition, bispecific, bispecific construct or fusion protein of the invention may be administered separately, simultaneously or sequentially with an anti-cancer agent.

Further anti-cancer agents that may be included in a combination therapy include immune check point inhibitors e.g. selected from PD-1 inhibitors, such as pembrolizumab, (Keytruda) and nivolumab (Opdivo), PD-L1 inhibitors, such as atezolizumab (Tecentriq), avelumab (Bavencio) and durvalumab (Imfinzi) and CTLA-4 inhibitors such as ipilimumab (Yervoy), other immune stimulants such as interferons (e.g. interferon α, β or γ), steroids e.g. prednisolone and alkylating agents such as platinum-based anti-neoplastic agents e.g. cisplatin, carboplatin and oxaliplatin.

In a preferred method of the invention said TCR, cell, clone or vector is administered in combination with an anti-cancer agent such as, but not limited to, a bispecific such as the bispecific of the invention.

### CLAUSES

Aspects of the invention include:

1. A cancer-specific T-cell receptor (TCR) or a cancer-specific binding fragment of a TCR which binds a tumour antigen, wherein the TCR or binding fragment comprises:
(a) an alpha chain comprising a CDR3α comprising or consisting of CAVRLAGYGGSQGNLIF, (SEQ ID NO: 1) or a variant CDR3α that has 1, 2, 3, 4 or 5 amino acid variations with respect thereto; and/or a beta chain comprising a CDR3β comprising or consisting of CASSSQGTDTQYF, (SEQ ID NO:2) or a variant CDR3β that has 1, 2, 3, 4 or 5 amino acid variations with respect thereto,
(b) an alpha chain comprising a CDR3α comprising or consisting of CALSSYHYGGSQGNLIF (SEQ ID NO: 33) or a variant CDR3α that has 1, 2, 3, 4 or 5 variations amino acid variations with respect thereto; and/or a beta chain comprising a CDR3β comprising or consisting of CASRGNTGELFF (SEQ ID NO: 36) or a variant CDR3β that has 1, 2, 3, 4 or 5 amino acid variations with respect thereto, or
(c) an alpha chain comprising a CDR3α comprising or consisting of CALSSYHYGGSQGNLIF (SEQ ID NO: 33) or a variant CDR3α that has 1, 2, 3, 4 or 5 variations with respect thereto; and/or a beta chain comprising a CDR3β comprising or consisting of CASRTGQGNQPQHF (SEQ ID NO: 37) or a variant CDR3β that has 1, 2, 3, 4 or 5 amino acid variations with respect thereto,
(d) an alpha chain comprising a CDR3α comprising or consisting of CAVREADYGGSQGNLIF (SEQ ID NO: 34) or a variant CDR3α that has 1, 2, 3, 4 or 5 variations with respect thereto; and/or a beta chain comprising a CDR3β comprising or consisting of CASSLEQGQYF (SEQ ID NO: 38) or a variant CDR3β that has 1, 2, 3, 4 or 5 amino acid variations with respect thereto, or
(e) an alpha chain comprising a CDR3α comprising or consisting of CALSSYHYGGSQGNLIF (SEQ ID NO: 33) or a variant CDR3α that has 1, 2, 3, 4 or 5 amino acid variations with respect thereto, such as 1 or 2 variations, such as 1 variation; and/or a beta chain comprising a CDR3β comprising or consisting of CASSLEQGQYF (SEQ ID NO: 38) or a variant CDR3β that has 1, 2, 3, 4 or 5 amino acid variations with respect thereto, wherein in each case the variations are selected from additions, substitutions and deletions.

2. The TCR or cancer-specific binding fragment of a TCR according to clause 1 wherein the TCR or binding fragment comprises:
(a) an alpha chain comprising a CDR3α comprising or consisting of CAVRLAGYGGSQGNLIF, (SEQ ID NO: 1) or a variant CDR3α that has 1, 2, 3, 4 or 5 amino acid variations with respect thereto; and a beta chain comprising a CDR3β comprising or consisting of CASSSQGTDTQYF, (SEQ ID NO:2) or a variant CDR3β that has 1, 2, 3, 4 or 5 amino acid variations with respect thereto,
(b) an alpha chain comprising a CDR3α comprising or consisting of CALSSYHYGGSQGNLIF (SEQ ID NO: 33) or a variant CDR3α that has 1, 2, 3, 4 or 5 variations amino acid variations with respect thereto; and a beta chain comprising a CDR3β comprising or consisting of CASRGNTGELFF (SEQ ID NO: 36) or a variant CDR3β that has 1, 2, 3, 4 or 5 amino acid variations with respect thereto, or
(c) an alpha chain comprising a CDR3α comprising or consisting of CALSSYHYGGSQGNLIF (SEQ ID NO: 33) or a variant CDR3α that has 1, 2, 3, 4 or 5 variations with respect thereto; and a beta chain comprising a CDR3β comprising or consisting of CASRTGQGNQPQHF (SEQ ID NO: 37) or a variant CDR3β that has 1, 2, 3, 4 or 5 amino acid variations with respect thereto,
(d) an alpha chain comprising a CDR3α comprising or consisting of CAVREADYGGSQGNLIF (SEQ ID NO: 34) or a variant CDR3α that has 1, 2, 3, 4 or 5 variations with respect thereto; and a beta chain comprising a CDR3β comprising or consisting of CASSLEQGQYF (SEQ ID NO: 38) or a variant CDR3β that has 1, 2, 3, 4 or 5 amino acid variations with respect thereto, or
(e) an alpha chain comprising a CDR3α comprising or consisting of CALSSYHYGGSQGNLIF (SEQ ID NO: 33) or a variant CDR3α that has 1, 2, 3, 4 or 5 amino acid variations with respect thereto; and a beta chain comprising a CDR3β comprising or consisting of CASSLEQGQYF (SEQ ID NO: 38) or a variant CDR3β that has 1, 2, 3, 4 or 5 amino acid variations with respect thereto, wherein in each case the variations are selected from additions, substitutions and deletions.

3. The TCR or a cancer-specific binding fragment of a TCR according to clause 2, wherein the TCR or binding fragment comprises:
(a) a CDR3α comprising or consisting of CAVRLAGYGGSQGNLIF, (SEQ ID NO: 1) and a CDR3β comprising or consisting of CASSSQGTDTQYF, (SEQ ID NO:2),
(b) a CDR3α comprising or consisting of CALSSYHYGGSQGNLIF (SEQ ID NO: 33) and a CDR3β comprising or consisting of CASRGNTGELFF (SEQ ID NO: 36),
(c) a CDR3α comprising or consisting of CALSSYHYGGSQGNLIF (SEQ ID NO: 33) and a CDR3β comprising or consisting of CASRTGQGNQPQHF (SEQ ID NO: 37),
(d) a CDR3α comprising or consisting of CAVREADYGGSQGNLIF (SEQ ID NO: 34) and a CDR3β comprising or consisting of CASSLEQGQYF (SEQ ID NO: 38), or
(e) a CDR3α comprising or consisting of CALSSYHYGGSQGNLIF (SEQ ID NO: 33) and a CDR3β comprising or consisting of CASSLEQGQYF (SEQ ID NO: 38).

4. The TCR or cancer-specific binding fragment of a TCR according to any one of clauses 1 to 3, wherein the TCR or binding fragment comprises:
(a) one or more of the following CDRs comprising or consisting of; DSAIYN, (SEQ ID NO: 3), IQSSQREQ, (SEQ ID NO: 4), SGHDY, (SEQ ID NO: 5), and FNNNVP, (SEQ ID NO: 6), or a variant CDR that has 1 or 2 amino acid variations, such as 1 variation with respect thereto,
(b) one or more of the following CDRs comprising or consisting of; ATGYPS, (SEQ ID NO: 39), ATKADDK, (SEQ ID NO: 40), SGHDY, (SEQ ID NO: 5), and FNNNVP, (SEQ ID NO: 6), or a variant CDR that has 1 or 2 amino acid variations, such as 1 variation with respect thereto,
(c) one or more of the following CDRs comprising or consisting of; ATGYPS, (SEQ ID NO: 39), ATKADDK, (SEQ ID NO: 40), SGHDY, (SEQ ID NO: 5), and FNNNVP, (SEQ ID NO: 6), or a variant CDR that has 1 or 2 amino acid variations, such as 1 variation with respect thereto
(d) one or more of the following CDRs comprising or consisting of; VGISA, (SEQ ID NO: 41), LSSGK, (SEQ ID NO: 42), MDHEN, (SEQ ID NO: 43), and SYDVKM, (SEQ ID NO: 44), or a variant CDR that has 1 or 2 amino acid variations, such as 1 variation with respect thereto; or
(e) one or more of the following CDRs comprising or consisting of; ATGYPS, (SEQ ID NO: 39), ATKADDK, (SEQ ID NO: 40), MDHEN, (SEQ ID NO: 43), and SYDVKM, (SEQ ID NO: 44); or a variant CDR that has 1 or 2 amino acid variations, such as 1 variation with respect thereto; wherein in each case the variations are selected from additions, substitutions and deletions.

5. The TCR or cancer-specific binding fragment of a TCR according to clause 4, wherein the TCR or binding fragment comprises an α chain with CDRs comprising or consisting of:
(a) CAVRLAGYGGSQGNLIF, (SEQ ID NO: 1) or a variant CDR that has 1, 2, 3, 4 or 5 amino acid variations with respect thereto; and DSAIYN, (SEQ ID NO: 3); and IQSSQREQ, (SEQ ID NO: 4) or a variant CDR that has 1 or 2 amino acid variations with respect thereto, such as 1 variation;
(b) CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation,
(c) CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation,
(d) CAVREADYGGSQGNLIF (SEQ ID NO: 34) or a variant CDR that has 1, 2, 3, 4 or 5 amino acid variations with respect thereto; and VGISA, (SEQ ID NO: 41) and LSSGK, (SEQ ID NO: 42) or a variant CDR that has 1 or 2 amino acid variations with respect thereto, such as 1 variation; or
(e) CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation; wherein in each case the variations are selected from additions, substitutions and deletions.

6. The TCR or cancer-specific binding fragment of a TCR according to clause 4 or clause 5, wherein the TCR or binding fragment comprises a β chain with CDRs comprising or consisting of:
(a) CASSSQGTDTQYF, (SEQ ID NO: 2) or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation,
(b) CASRGNTGELFF (SEQ ID NO: 36), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation,
(c) CASRTGQGNQPQHF (SEQ ID NO: 37), or a variant CDR that has 1, 2, 3, 4 or 5 amino acid variations with respect thereto, and SGHDY (SEQ ID NO: 5) and FNNNVP (SEQ ID NO: 6) or a variant CDR that has 1 or 2 amino acid variations with respect thereto, such as 1 variation,
(d) CASSLEQGQYF (SEQ ID NO: 38), or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations, such as 1 variation; or
(e) CASSLEQGQYF (SEQ ID NO: 38) or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; wherein in each case the variations are selected from additions, substitutions and deletions.

7. The TCR or cancer-specific binding fragment of a TCR according to clause 1, wherein the TCR or binding fragment comprises:
(a) 3 α-chain CDRs comprising or consisting of CAVRLAGYGGSQGNLIF, (SEQ ID NO: 1) or a variant CDR that has 1, 2, 3, 4 or 5 amino acid variations with respect thereto; DSAIYN, (SEQ ID NO: 3) and IQSSQREQ, (SEQ ID NO: 4) or a variant CDR that has 1 or 2 amino acid variations with respect thereto, such as 1 variation; and comprises 3 β-chain CDRs comprising or consisting of CASSSQGTDTQYF, (SEQ ID NO: 2) or a variant CDR that has 1, 2, 3, 4 or 5 amino acid variations with respect thereto, and SGHDY, (SEQ ID NO: 5) and FNNNVP, (SEQ ID NO: 6) or a variant CDR that has 1 or 2 amino acid variations with respect thereto, such as 1 variation,
(b) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRGNTGELFF (SEQ ID NO: 36), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation;
(c) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRTGQGNQPQHF (SEQ ID NO: 37), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation;
(d) 3 α-chain CDRs comprising or consisting of CAVREADYGGSQGNLIF (SEQ ID NO: 34) or a variant CDR that has 1, 2, 3, 4 or 5 amino acid variations with respect thereto; and VGISA, (SEQ ID NO: 41) and LSSGK, (SEQ ID NO: 42) or a variant CDR that has 1 or 2 amino acid variations with respect thereto, such as 1 variation; and comprises 3-β chain CDRs comprising or consisting of CASSLEQGQYF (SEQ ID NO: 38), or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; or
(e) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation and comprises 3-β chain CDRs comprising or consisting of CASSLEQGQYF (SEQ ID NO: 38) or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; wherein in each case the variations are selected from additions, substitutions and deletions.

8. The TCR or cancer-specific binding fragment of a TCR according to any one of clauses 1 to 7, wherein the TCR or binding fragment is not expressed by or associated with a mucosal-associated invariant T-cell (MAIT cell).

9. The TCR or cancer-specific binding fragment of a TCR according to any one of clauses 1 to 8, wherein the TCR comprises an α chain extracellular region comprising or consisting of:

(a)
(SEQ ID NO: 9)
KQEVTQIPAALSVPEGENLVLNCSFTDSAIYNLQWFRQDPGKGLTSLLL
IQSSQREQTSGRLNASLDKSSGRSTLYIAASQPGDSATYLCAVRLAGYG
GSQGNLIFGKGTKLSVKPNIQNPDPAVYQLRDSKSSDKSVCLFTDFDSQ
TNVSQSKDSDVYITDKTVLDMRSMDFKSNSAVAWSNKSDFACANAFNNS
IIPEDTFFPSPESSCDVKLVEKSFETDTNLNFQNLSVIGFR.,
(b)
(SEQ ID NO: 53)
DSVTQMEGPVTLSEEAFLTINCTYTATGYPSLFWYVQYPGEGLQLLLKA
TKADDKGSNKGFEATYRKETTSFHLEKGSVQVSDSAVYFCALSSYHYGG
SQGNLIFGKGTKLSVKPNIQNPDPAVYQLRDSKSSDKSVCLFTDFDSQT
NVSQSKDSDVYITDKTVLDMRSMDFKSNSAVAWSNKSDFACANAFNNSI
IPEDTFFPSPESSCDVKLVEKSFETDTNLNFQNLSVIGFR,
(c)
(SEQ ID NO: 55)
DSVTQMEGPVTLSEEAFLTINCTYTATGYPSLFWYVQYPGEGLQLLLKA
TKADDKGSNKGFEATYRKETTSFHLEKGSVQVSDSAVYFCALSSYHYGG
SQGNLIFGKGTKLSVKPNIQNPDPAVYQLRDSKSSDKSVCLFTDFDSQT
NVSQSKDSDVYITDKTVLDMRSMDFKSNSAVAWSNKSDFACANAFNNSI
IPEDTFFPSPESSCDVKLVEKSFETDTNLNFQNLSVIGFR,
(d)
(SEQ ID NO: 57)
NEVEQSPQNLTAQEGEFITINCSYSVGISALHWLQQHPGGGIVSLFMLS
SGKKKHGRLIATINIQEKHSSLHITASHPRDSAVYICAVRLAGYGGSQG
NLIFGKGTKLSVKPNIQNPDPAVYQLRDSKSSDKSVCLFTDFDSQTNVS
QSKDSDVYITDKTVLDMRSMDFKSNSAVAWSNKSDFACANAFNNSIIPE
DTFFPSPESSCDVKLVEKSFETDTNLNFQNLSVIGFR,;
or
(e)
(SEQ ID NO: 59)
DSVTQMEGPVTLSEEAFLTINCTYTATGYPSLFWYVQYPGEGLQLLLKA
TKADDKGSNKGFEATYRKETTSFHLEKGSVQVSDSAVYFCALSSYHYGG
SQGNLIFGKGTKLSVKPNIQNPDPAVYQLRDSKSSDKSVCLFTDFDSQT
NVSQSKDSDVYITDKTVLDMRSMDFKSNSAVAWSNKSDFACANAFNNSI
IPEDTFFPSPESSCDVKLVEKSFETDTNLNFQNLSVIGFR,;

or a variant α chain extracellular region which has at least 85% sequence identity to the α chain extracellular region of SEQ ID NO: 9, 53, 55, 57 or 59.

10. The TCR or cancer-specific binding fragment of a TCR according to clause 9, wherein said TCR comprises an α chain extracellular region comprising or consisting of: SEQ ID NO: 9, 53, 55, 57 or 59.

11. The TCR or cancer-specific binding fragment of a TCR according to any one of clauses 1 to 10, wherein said TCR comprises a β chain extracellular region comprising or consisting of:

(a)
(SEQ ID NO: 10)
DAGVIQSPRHEVTEMGQEVTLRCKPISGHDYLFWYRQTMMRGLELLIYF
NNNVPIDDSGMPEDRFSAKMPNASFSTLKIQPSEPRDSAVYFCASSSQG
TDTQYFGPGTRLTVLEDLKNVFPPKVAVFEPSEAEISHTQKATLVCLAT
GFYPDHVELSWWVNGKEVHSGVSTDPQPLKEQPALNDSRYCLSSRLRVS
ATFWQNPRNHFRCQVQFYGLSENDEWTQDRAKPVTQIVSAEAWGRADCG
FTSESYQQGVLSATILYE.,
(b)
(SEQ ID NO: 54)
GVIQSPRHEVTEMGQEVTLRCKPISGHDYLFWYRQTMMRGLELLIYFNN
NVPIDDSGMPEDRFSAKMPNASFSTLKIQPSEPRDSAVYFCASSLCASR
GNTGELFFGEGSRLTVLEDLKNVFPPKVAVFEPSEAEISHTQKATLVCL
ATGFYPDHVELSWWVNGKEVHSGVSTDPQPLKEQPALNDSRYCLSSRLR
VSATFWQNPRNHFRCQVQFYGLSENDEWTQDRAKPVTQIVSAEAWGRAD
CGFTSESYQQGVLSATILYE,,
(c)
(SEQ ID NO: 56)
GVIQSPRHEVTEMGQEVTLRCKPISGHDYLFWYRQTMMRGLELLIYFNN
NVPIDDSGMPEDRFSAKMPNASFSTLKIQPSEPRDSAVYFCASSLCASR
TGQGNQPQHFGDGTRLSILEDLKNVFPPKVAVFEPSEAEISHTQKATLV
CLATGFYPDHVELSWWVNGKEVHSGVSTDPQPLKEQPALNDSRYCLSSR
LRVSATFWQNPRNHFRCQVQFYGLSENDEWTQDRAKPVTQIVSAEAWGR
ADCGFTSESYQQGVLSATILYE,,
(d)
(SEQ ID NO: 58)
SRYLVKRTGEKVFLECVQDMDHENMFWYRQDPGLGLRLIYFSYDVKMKE
KGDIPEGYSVSREKKERFSLILESASTNQTSMYLCASSLCASSLEQGQY
FGPGTRLLVLEDLKNVFPPKVAVFEPSEAEISHTQKATLVCLATGFYPD
HVELSWWVNGKEVHSGVSTDPQPLKEQPALNDSRYCLSSRLRVSATFWQ
NPRNHFRCQVQFYGLSENDEWTQDRAKPVTQIVSAEAWGRADCGFTSES
YQQGVLSATILYE,;
or
(e)
(SEQ ID NO: 60)
SRYLVKRTGEKVFLECVQDMDHENMFWYRQDPGLGLRLIYFSYDVKMKE
KGDIPEGYSVSREKKERFSLILESASTNQTSMYLCASSLEQGQYFGPGT
RLLVLEDLKNVFPPKVAVFEPSEAEISHTQKATLVCLATGFYPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADCGFTSESYQQGV
LSATILYE,;

or a variant β chain extracellular region which has at least 85% sequence identity to the β chain extracellular region of SEQ ID NO: 10, 54, 56, 58 or 60.

12. The TCR or cancer-specific binding fragment of a TCR according to any one of clauses 1 to 11, wherein said TCR comprises a β chain extracellular region comprising or consisting of: SEQ ID NO: 10, 54, 56, 58 or 60.

13. The TCR or cancer-specific binding fragment of a TCR according to any one of clauses 1 to 12, wherein said TCR comprises:
(a) an α chain extracellular region comprising or consisting of SEQ ID NO: 9 and a β chain extracellular region comprising or consisting of SEQ ID NO: 10, or a variant α and/or β chain extracellular region which has at least 85% sequence identity thereto respectively,
(b) an α chain extracellular region comprising or consisting of SEQ ID NO: 53 and a β chain extracellular region comprising or consisting of SEQ ID NO: 54, or a variant α and/or β chain extracellular region which has at least 85% sequence identity thereto respectively,
(c) an α chain extracellular region comprising or consisting of SEQ ID NO: 55 and a β chain extracellular region comprising or consisting of SEQ ID NO: 56, or a variant α and/or β chain extracellular region which has at least 85% sequence identity thereto respectively,
(d) an α chain extracellular region comprising or consisting of SEQ ID NO: 57 and a β chain extracellular region comprising or consisting of SEQ ID NO: 58, or a variant α and/or β chain extracellular region which has at least 85% sequence identity thereto respectively, or
(e) an α chain extracellular region comprising or consisting of SEQ ID NO: 59 and a β chain extracellular region comprising or consisting of SEQ ID NO: 60 or a variant α and/or β chain extracellular region which has at least 85% sequence identity thereto respectively.

14. The TCR according to clause 1 comprising:
(a) an α chain comprising a CDR3 sequence of SEQ ID NO: 1 or a variant CDR3 that has 1, 2 or 3 amino acid variations with respect thereto, such as 1 or 2 variations such as 1 variation, a CDR1 sequence of SEQ ID NO:3 or a variant CDR1 that has 1 or 2 amino acid variations with respect thereto, such as 1 variation and CDR2 sequence of SEQ ID NO: 4 or a variant CDR2 that has 1 or 2 amino acid variations with respect thereto, such as 1 variation and a β chain comprising a CDR3 sequence of SEQ ID NO: 2 or a variant CDR3 that has 1, 2 or 3 amino acid variations with respect thereto, such as 1 or 2 variations such as 1 variation, a CDR1 sequence of SEQ ID NO: 5 or a variant CDR1 that has 1 or 2 amino acid variations with respect thereto, such as 1 variation and CDR2 sequence of SEQ ID NO: 6 or a variant CDR2 that has 1 or 2 amino acid variations with respect thereto, such as 1 variation; or
(b) an α chain extracellular region comprising or consisting of SEQ ID NO: 11 or a variant α chain extracellular region which has at least 85% sequence identity to the α chain extracellular region of SEQ ID NO: 11 and a β chain extracellular region comprising SEQ ID NO: 12 or a variant β chain extracellular region which has at least 85% sequence identity to the β chain extracellular region of SEQ ID NO: 12 or a β chain extracellular region comprising SEQ ID NO: 13 or a variant β chain extracellular region which has at least 85% sequence identity to the β chain extracellular region of SEQ ID NO: 13; or
(c) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising or consisting of SEQ ID NO: 10 or a variant β chain extracellular region which has at least 85% sequence identity to the β chain extracellular region of SEQ ID NO: 10;
(d) an α chain comprising or consisting of SEQ ID NO: 29 or a variant α chain which has at least 85% sequence identity to the α chain of SEQ ID NO: 29 and a β chain comprising or consisting of SEQ ID NO: 31 or a variant β chain which has at least 85% sequence identity to the β chain of SEQ ID NO: 31; or
(e) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising SEQ ID NO: 12 or a variant β chain extracellular region which has at least 85% sequence identity to the β chain extracellular region of SEQ ID NO: 12 or a β chain extracellular region comprising SEQ ID NO: 13 or a variant β chain extracellular region which has at least 85% sequence identity to the @ chain extracellular region of SEQ ID NO: 13; or
(f) an α chain variable region comprising or consisting of SEQ ID NO: 14 or a variant α chain variable region which has at least 85% sequence identity to the α chain variable region of SEQ ID NO: 14 and a β chain variable region comprising or consisting of SEQ ID NO: 15 or a variant β chain variable region which has at least 85% sequence identity to the β chain variable region of SEQ ID NO: 15; or
(g) an α chain variable region comprising or consisting of SEQ ID NO: 45 or a variant α chain variable region which has at least 85% sequence identity to the α chain variable region of SEQ ID NO: 45 and a β chain variable region comprising or consisting of SEQ ID NO: 46 or a variant β chain variable region which has at least 85% sequence identity to the β chain variable region of SEQ ID NO: 46; or
(h) an α chain variable region comprising or consisting of SEQ ID NO: 47 or a variant α chain variable region which has at least 85% sequence identity to the α chain variable region of SEQ ID NO: 47 and a β chain variable region comprising or consisting of SEQ ID NO: 48 or a variant β chain variable region which has at least 85% sequence identity to the β chain variable region of SEQ ID NO: 48; or
(i) an α chain variable region comprising or consisting of SEQ ID NO: 49 or a variant α chain variable region which has at least 85% sequence identity to the α chain variable region of SEQ ID NO: 49 and a β chain variable region comprising or consisting of SEQ ID NO: 50 or a variant β chain variable region which has at least 85% sequence identity to the β chain variable region of SEQ ID NO: 50; or
(j) an α chain variable region comprising or consisting of SEQ ID NO: 51 or a variant α chain variable region which has at least 85% sequence identity to the α chain variable region of SEQ ID NO: 51 and a β chain variable region comprising or consisting of SEQ ID NO: 52 or a variant β chain variable region which has at least 85% sequence identity to the β chain variable region of SEQ ID NO: 52; optionally wherein in each case the variations are selected from additions, substitutions and deletions.

15. The TCR or cancer-specific binding fragment of a TCR according to any one of clauses 1 to 14, wherein the amino acid sequence of the TCR or binding fragment is artificial.

16. The TCR or cancer-specific binding fragment of a TCR according to clause 15, wherein at least one amino acid is substituted, added or deleted relative to the wildtype or respective stated sequence.

17. The TCR or cancer-specific binding fragment of a TCR according to clause 16, wherein the at least one amino acid is (are) located in a framework region, a CDR or a constant region.

18. The TCR or cancer-specific binding fragment of a TCR according to clause 17, wherein the at least one amino acid is not located in any CDR.

19. The TCR or cancer-specific binding fragment of a TCR according to any one of clauses 1 to 18, which is a soluble form of a TCR.

20. The TCR or cancer-specific binding fragment of a TCR according to any one of clauses 1 to 19 wherein the TCR or cancer-specific binding fragment is MR1-restricted.

21. The TCR or cancer-specific binding fragment of a TCR according to any one of clauses 1 to 20 comprising an α chain and a β chain each comprising a constant region in which the constant region of one or both chains of the said TCR or cancer-specific binding fragment of a TCR is modified.

22. A polynucleotide encoding the TCR or cancer-specific binding fragment according to any one of clauses 1 to 21.

23. The polynucleotide according to clause 22 encoding two chains of the TCR or cancer-specific binding fragment therefore connected by a cleavable linker.

24. A polynucleotide according to clause 22 or 23, which is a chimeric polynucleotide comprising a gene encoding the TCR or cancer-specific binding fragment and a heterologous promoter or other transcription control element operably linked thereto.

25. A vector for delivery of a polynucleotide to cells comprising a polynucleotide according to any one of clauses 22 to 24.

26. The vector according to clause 25, wherein the vector is a viral vector.

27 The vector according to clause 26, wherein the viral vector is a lentiviral vector.

28. An immune cell, such as a T-cell, particularly an engineered immune cell such as an engineered T-cell, expressing the TCR or cancer-specific binding fragment according to any one of clauses 1 to 21 or comprising the polynucleotide or vector of clauses 22 to 27.

29. An immune cell clone, such as a T-cell clone, particularly an engineered immune cell clone such as an engineered T-cell clone, expressing the TCR or cancer-specific binding fragment according to any one of clauses 1 to 21.

30. The immune cell clone, such as a T-cell clone according to clause 29 wherein the clone is a MC.27.759S, A3, A4, C1, or C2 clone.

31. The immune cell, such as a T-cell or immune cell clone, such as a T-cell clone according to any one of clauses 28 to 30, wherein the cell or cells of the clone are modified by the introduction (e.g. by transduction) of a heterologous polynucleotide according to clauses 22 to 24 or a vector according to any one of clauses 25 to 27.

32. An ex vivo process comprising (i) obtaining immune cells, particularly T-cells from a patient, (ii) optionally expanding the immune cells, particularly T-cells (iii) introducing a heterologous polynucleotide according to clauses 22 to 24 or a vector according to any one of clauses 25 to 27 into the immune cells, particularly T-cells to produce modified immune cells, particularly T-cells which express a TCR or a cancer-specific binding fragment of a TCR according to any one of clauses 1 to 21; and (iii) reintroducing said modified immune cells, particularly T-cells into the patient.

33. A method of treatment of cancer comprising administering to a patient in need thereof immune cells, particularly T-cells wherein the immune cells, particularly T-cells are immune cells, particularly T-cells which have been obtained from said patient and a heterologous polynucleotide according to clauses 22 to 24 or a vector according to any one of clauses 25 to 27 has been introduced into the immune cells, particularly T-cells, such that the immune cells, particularly T-cells express a cancer-specific TCR or a cancer-specific binding fragment of a TCR according to any one of clauses 1 to 21.

34. A pharmaceutical composition comprising the TCR or cancer-specific binding fragment according to any one of clauses 1 to 21, the polynucleotide according to clauses 22 to 24, a vector according to any one of clauses 25 to 27, the immune cell or the immune cell clone, particularly the T-cell or the T-cell clone according to any one of clauses 28 to 30 and a pharmaceutically acceptable carrier.

35. A pharmaceutical composition according to clause 34, wherein the pharmaceutical composition is formulated under sterile conditions and is suitable for parenteral administration.

36. A bispecific construct comprising the TCR or cancer-specific binding fragment according to any one of clauses 1 to 21 and an immune cell activating component or ligand that binds to and activates an immune cell.

37. A bispecific construct according to clause 36, wherein the immune cell activating component or ligand that binds to and activates an immune cell binds to CD3.

38. A fusion protein comprising the TCR or cancer-specific binding fragment according to any one of clauses 1 to 21 and a heterologous protein.

39. A method of treating cancer in a subject comprising administering a therapeutically effective amount of the immune cell, immune cell clone, T-cell or T-cell clone according to any one of clauses 28 to 30, pharmaceutical composition according to any one of clauses 34 to 35, bispecific construct according to clause 36 or 37 or fusion protein according to clause 38, to the subject.

40. The method according to any one of clauses 33 or 39 wherein said cancer is selected from lung, melanoma (e.g. skin melanoma), bone, breast, blood (e.g. leukemia), prostate, kidney, bladder, cervical, ovarian and colorectal cancers, and in particular selected from lung, melanoma (e.g. skin melanoma), bone and breast cancers.

41. The method according to any one of clauses 33, 39 or 40, wherein the immune cell, immune cell clone, T-cell, T-cell clone, pharmaceutical composition, bispecific construct or fusion protein is administered separately, simultaneously or sequentially with an anti-cancer agent.

42. A pharmaceutical composition comprising:
a) the immune cell, immune cell clone, T-cell or T-cell clone according to any one of clauses 28 to 30, the pharmaceutical composition according to clauses 34 or 35, bispecific construct according to clause 36 or 37 or the fusion protein according to clause 38; and
b) an anti-cancer agent.

In the claims which follow and in the preceding description of the invention, except where the context requires otherwise due to express language or necessary implication, the word “comprises”, or variations such as “comprised or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

### EXAMPLES

### Example 1

### Identification and Cloning of MR1-Reactive T-Cells

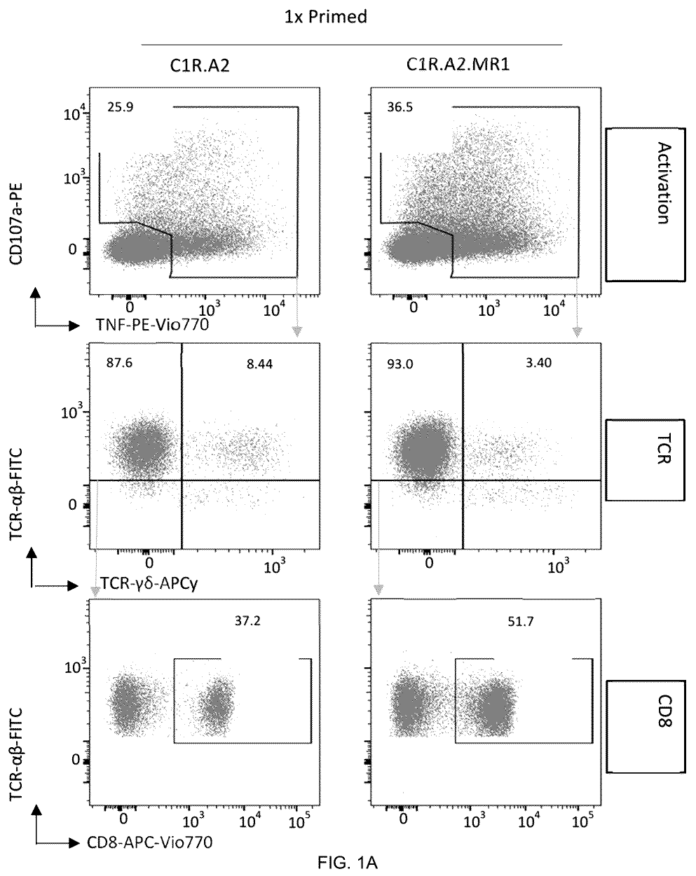

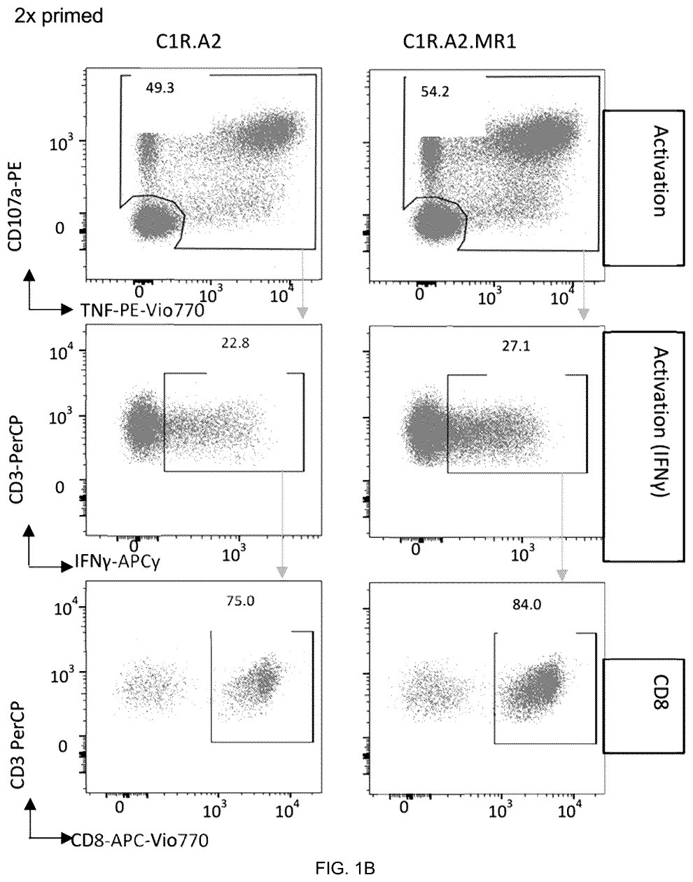

To identify if MR1-reactive T-cells are present in healthy donors, priming of HLA-A2-negative PBMCs with C1R.A2 cells transduced with MR1 (C1R.A2.MR1) was performed. C1R.A2.MR1 target cells were irradiated prior to T-cell priming to prevent their uncontrolled outgrowth in priming cultures. After two weeks of PBMC/C1R.A2.MR1 co-culture, expansion of MR1-reactive T-cells was enumerated by performing a TAPI/CD107a assay on the cultures using C1R.A2 and C1R.A2.MR1 cells. MR1-reactive T-cells were enriched in the healthy donor PBMCs, however there was high background to the C1R.A2 cells, likely through allogenic recognition (FIG. 1A). A second round of priming on the T-cells, included labelling for IFN-γ did not reveal further MR1-specific enrichment (FIG. 1B).

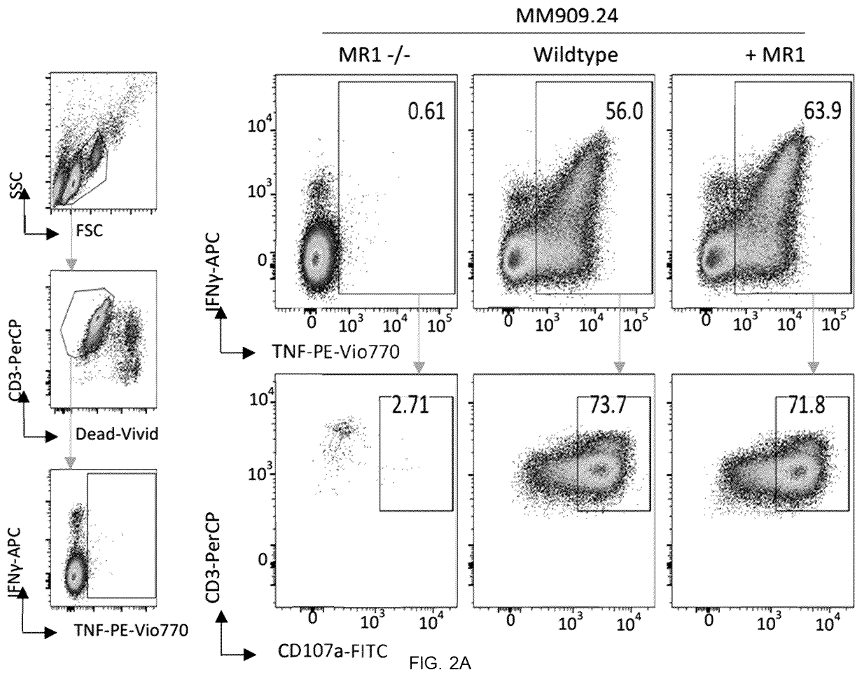

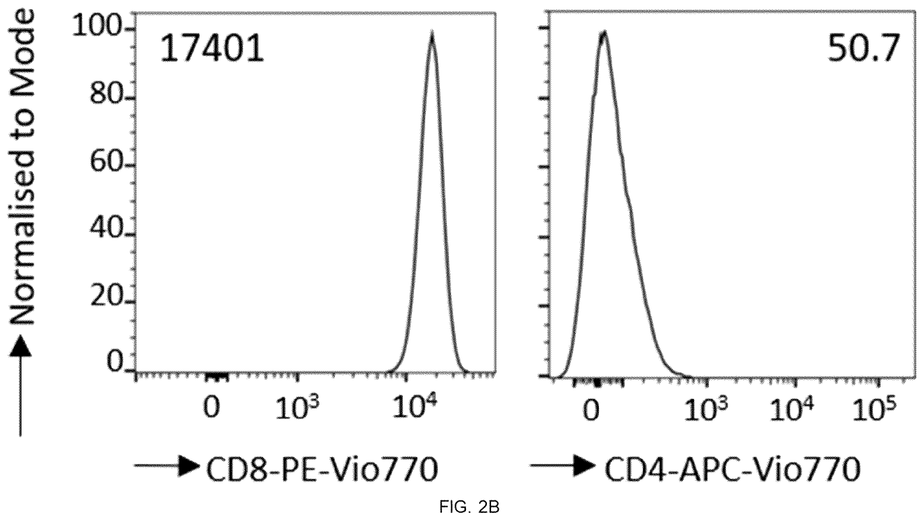

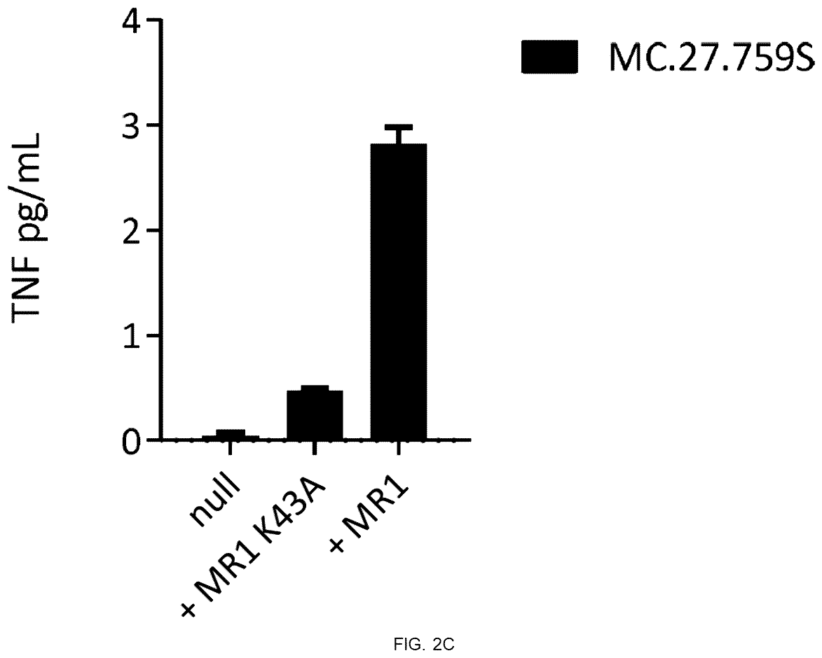

To further enrich MR1-reactive cells, C1R cells were transduced with MR1 (C1R.MR1), as these are HLA-A and B negative (Storkus et al. 1989) and do not induce a large HLA class-I antigen specific response. The previously described MR1-reactive T-cell clone MC.7.G5 (see WO2019/097244; referred to as “MC.7.G5”) recognises both C1R.MR1 cells and a melanoma cell line derived from a melanoma patient MM909.24 (referred to as “MM909.24”) transfected with MR1 (MM909.24+MR1). T-cell clone, designated MC.27.759S, was isolated by flow cytometry sorting on TNFα+CD107a+ T-cells in response to C1R.MR1 cells from donor 759S (FIG. 1A and FIG. 1B). MC.27.759S was able to secrete IFNγ and TNFα cytokines and express CD107a in response to C1R.MR1, MM909.24+MR1 and MM909.24 wildtype (FIG. 2A). MC.27.759S exhibited a high expression of CD8 and almost undetectable CD4 (FIG. 2B). When exposed to C1R cells overexpressing MR1 with a K43A mutation, MC.27.759S was minimally stimulated (FIG. 2C). MC.27.759S therefore appears to show ligand specificity in the context of MR1.

### Example 2

### The Sequence of the MC.27.759S TCR α and β Chains.

The sequences of the MC.27.759S TCR α and β chains was elucidated as follows. RNA was extracted using the RNEasy Micro kit (Qiagen). cDNA was synthesized using the 5′/3′ SMARTer kit (Clontech, Paris, France) according to the manufacturer's instructions. The SMARTer approach used a Murine Moloney Leukaemia Virus (MMLV) reverse transcriptase, a 3′ oligo-dT primer and a 5′ oligonucleotide to generate cDNA templates, which were flanked by a known, universal anchor sequence. PCR was then set up using a single primer pair. A TCR-β constant region-specific reverse primer (Cβ-R1, 5′-GAGACCCTCAGGCGGCTGCTC-3′, SEQ ID NO: 18, Eurofins Genomics, Ebersberg, Germany) and an anchor-specific forward primer (Clontech) were used in the following PCR reaction: 2.5 μL template cDNA, 0.25 μL High Fidelity Phusion Taq polymerase, 10 μL 5× Phusion buffer, 0.5 μL DMSO (all from Thermo Fisher Scientific), 1 μL dNTP (50 mM each, Life Technologies), 1 μL of each primer (10 μM), and nuclease-free water for a final reaction volume of 50 μL. Subsequently, 2.5 μL of the first PCR products were taken out to set up a nested PCR as above, using a nested primer pair (Cβ-R2, 5′-TGTGTGGCCAGGCACACCAGTGTG-3, SEQ ID NO: 19, Eurofins Genomics and anchor-specific primer from Clontech). For both PCR reactions, cycling conditions were as follows: 94° C. for 5 min, 30 cycles of 94° C. for 30 s, 63° C. for 30 s, 72° C. for 90s, and finally 72° C., for 10 min. The final PCR products were loaded on a 1% agarose gel and purified with the QIAEX II gel extraction kit (Qiagen). Purified products were cloned into Zero-Blunt TOPO and transformed into One Shot Chemically Competent E. coli cells for standard sequencing (both from Life Technologies). The TCR alpha chain was found to be comprised of TRAV21/TRAJ42 TCRα chain segments with a CDR3 sequence CAVRLAGYGGSQGNLIF. The TCR β chain was comprised of TRBV9/TRDB2/TRBJ2.3 segments with a CDR3 sequence CASSVASTGTDTQYF. The sequences of the α and β chain of the MC.27.759S clone are SEQ ID NO: 7 and 8, respectively.

### Example 3

Clone MC.27.759S Responds to a Wide Range of Tumour Targets and does not Respond to Normal Cells

Activation assays were performed as above and also cytotoxicity assays using either sodium chromate (Chromium⁵¹) labelled target cells (Ekeruche-Makinde et al., 2012), or a flow cytometry based long-term killing assay. For chromium release assays each cell line was labelled with 30 μCi of Cr⁵¹ (Perkin Elmer, Waltham, MA) per 1×10⁶ cells and 2000 target cells used per well (96U well pates) with MC.27.759S to achieve the desired T-cell to target cell ratios. After overnight incubation the supernatants were harvested, mixed with scintillant and read using a Microbeta counter and specific lysis calculated as previously described (Ekeruche-Makinde et al., 2012). The details of the cancer cell lines used are as follows: cell line name (ATCC® reference or ECACC number for background and culture information)/tissue or organ of origin: A549 (lung carcinoma, CCL-185), MCF-7 (breast, HTB-22), and U20S (bone, HTB-96). MM909.11, MM909.15, MM909.24, MM909.29 and MM909.37 (all skin melanomas) were obtained from cancer patients treated at the Center for Cancer Immune Therapy (CCIT, Herlev Hospital, Copenhagen, Denmark).

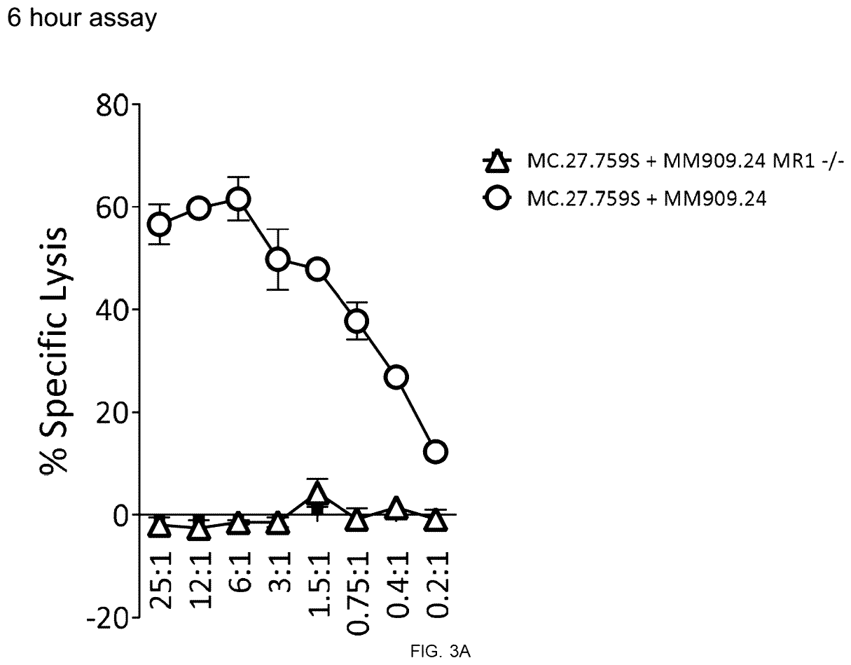

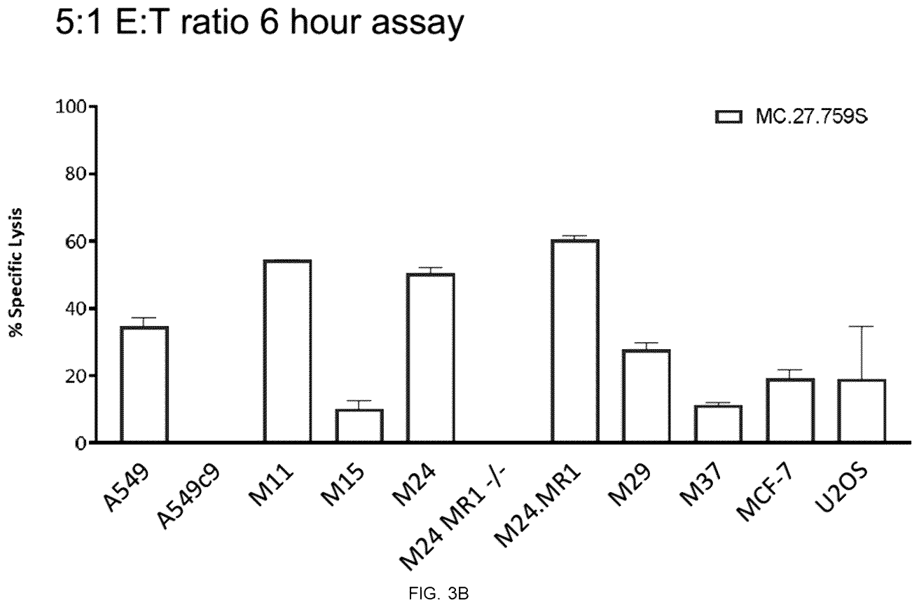

MC.27.759S does not recognise A549 cells through MHC-I or MHC-II, intimating recognition is not via classical MHC. MC.27.759S killing capacity was observed across a range of E: T ratios in a 6 hr killing assay against MM909.24 wildtype, while remaining inert to MM909.24 MR1−/− (FIG. 3A). A 6 hour chromium release assay with an effector: target ratio of 5:1 showed MC.27.759S could kill a range of cancer cell lines derived from lung, skin, breast and bone (FIG. 3B).

### Example 4

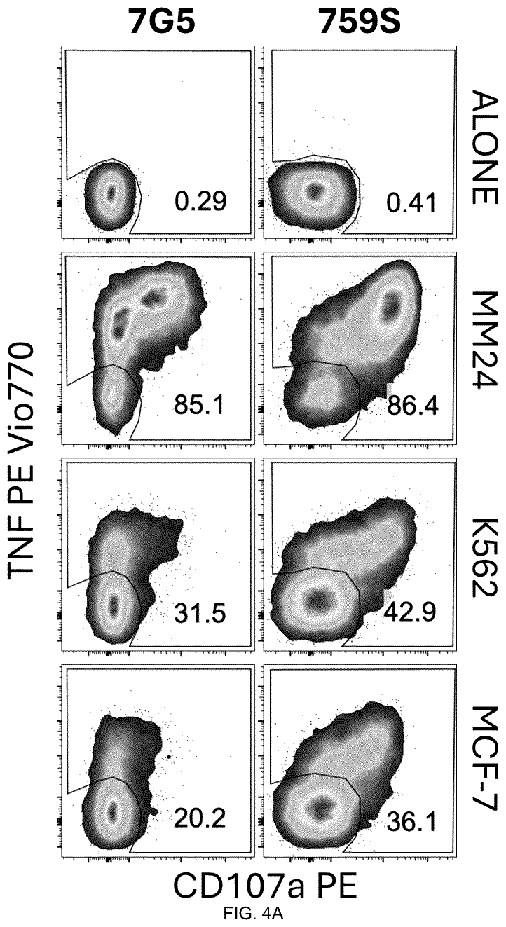

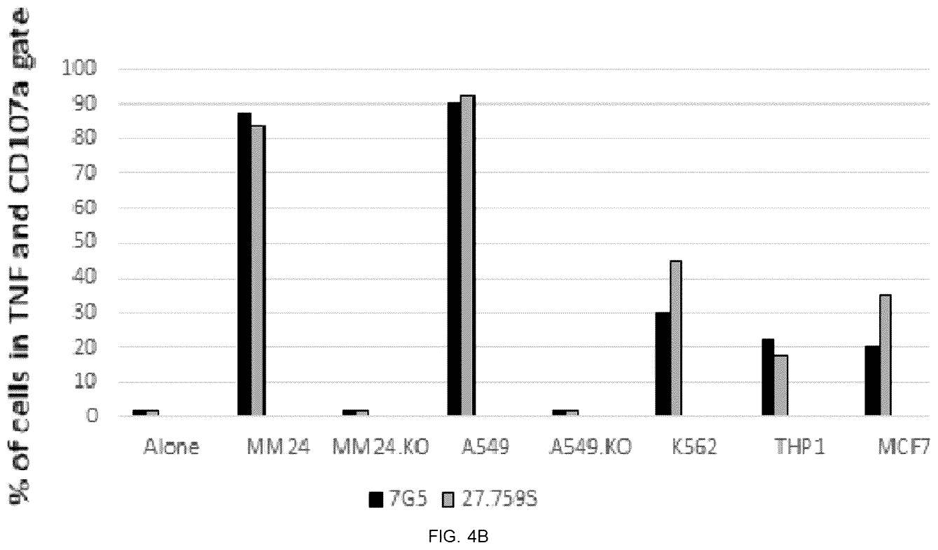

Comparison of T cell clones MC.7.G5 and MC.27.759S. T cells were rested in R5 (RPMI 1640 supplemented with 5% fetal bovine serum (FBS), 100 U ml⁻¹ penicillin, 100 μg ml⁻¹ streptomycin and 2 mM I-glutamine (Life Technologies) for 24 h before assay. Typically, 3×10⁴ T cells and 3×10⁴ target cells were used per well, inducing activation for 4 h at a T cell to target cell ratio of 1:1. For TNF-processing inhibitor TAPI-0 (Santa Cruz Biotechnology) assays, T cells and target cells were co-incubated for 4 h with 30 μM TAPI-0 and antibody directed against TNF (clone cA2, Miltenyi Biotec). CD107a antibody (clone H4A3, Miltenyi Biotec Ltd.) was also included at the start of the assay to detect activation-induced degranulation of cytotoxic T cells. Following incubation, cells were washed and stained with Fixable Live/Dead Violet Dye and antibodies against T cell surface markers. Gating strategy and isotype antibody (as recommended by the manufacturer of the primary antibodies) shown in FIG. 4A. Percentage of cells positive for TNFα and CD107a in the clones were calculated and are shown in as a barchart in FIG. 4B. Data indicates that MC.27.759S produces an activation-induced functional response to target a broad range of cancer cells with an effectiveness comparable to MC.7.G5, neither clone responds in absence of MR1 (knockout target cell data).

### Example 5

Clone MC.27.759S should not Stain with Tetrameric Forms of the MAIT Ligand MR1-5-OP-RU or with MR1-Ac-6-FP

Surface-expressed MR1 has previously been shown to induce a response in T-cells without a presented ligand; the K43A mutated form of MR1 disables Schiff-bond formation with MR1 ligand and enables antigen-independent MR1 refolding (Reantragoon et al. 2013). MR1-K43A is however still able to bind ligands that do not require Schiff bond formation such as the MAIT-activating ligand RL-6-Me-7-OH. Refolded MR1-K43A added ligand have been previously shown to bind to T-cells (Gherardin et al. 2016).

Therefore, to test if MC.27.759S simply recognised surface-expressed refolded MR1 independent of bound ligand, the binding of MR1 tetramers is assessed (MR1 monomers provided by Professor Jamie Rossjohn). MC.27.759S should not stain with MR1-K43A tetramer, nor MR1 tetramer loaded with the MAIT-activating ligand 5-OP-RU, whereas a conventional MAIT clone should stained only with MR1-5-OP-RU tetramer. An HLA-A2 tetramer loaded with the Melan-A derived peptide ELAGIGILTV (SEQ ID NO: 23) is used a negative control which should stain the cognate MEL5 Melan-A specific T-cell clone. C1R cells transduced with a lentivirus containing MR1 K43A, when cultured with MC.27.759S should not induce TNFα secretion unlike the native MR1 counterpart, despite increased surface MR1 expression. Thus, MC.27.759S should not recognise a MAIT ligand in MR1 or empty MR1; suggesting that this T-cell clone should recognise a cancer-specific ligand within the MR1 binding groove.

Data indicated that MC.27.759S appeared not to recognise or bind the MAIT ligand MR1-5-OP-RU, however the positive control results also indicated poor lack of MR1-5-OP-RU binding to the MAIT clone which indicated possible activity problems with the particular MAIT clone as control.

### Example 6

Ac-6-FP and M.Smeg Infection should Reduce Recognition by Clone MC.27.759S Despite Enhancing Expression of MR1 at the Cell Surface

To confirm that MC.27.759S recognised an MR1-presented ligand A549 cells are loaded with M. smegmatis, known to be a potent producer of MAIT-activating MR1 ligands (Laugel et al. 2016). The rationale for this experiment is that M. smegmatis-derived MAIT-activating MR1 ligands would compete for any cancer-specific ligand within these cells, thereby lowering recognition by MC.27.759S while increasing recognition by a canonical MAIT cell. M. smegmatis loading of A549 cells should result in a decrease in TNFα secretion and CD107a expression by MC.27.759S while inducing recognition by a conventional MAIT clone. Furthermore, the MR1 ligand Ac-6-FP, which does not activate conventional MAIT cells, has previously been used to verify the ligand specificity of MR1-restricted T-cells (Eckle et al. 2014; Lepore et al. 2017). Titration of Ac-6-FP on MM909.24 cells should block recognition. These data together should indicate that MC.27.759S does not simply recognise folded surface MR1, but rather MR1 with bound cargo that is associated with cancer cells.

### Example 7

Transduction of Polyclonal T-Cells with the MC.27.759S TCR

Codon-optimized, full-length TCR chains, separated by a self-cleaving 2A sequence, are synthesized (Genewiz) (SEQ ID NO: 16) corresponding to native TCR chain sequences SEQ ID NOs: 7 and 8) and cloned into the 3^{rd} generation lentiviral transfer vector pELNS (kindly provided by Dr. James Riley, University of Pennsylvania, PA). The pELNS vector contains a rat CD2 (rCD2) marker gene separated from the TCR by another self-cleaving 2A sequence. Lentiviral particles are generated by calcium chloride transfection of HEK293T cells. TCR transfer vectors are co-transfected with packaging and envelope plasmids pMD2.G, pRSV-Rev and pMDLg/pRRE. Lentiviral particles are 0.44-μm filtered then concentrated by ultracentrifugation (150,000 g for 2 h at 4° C.) prior to transduction of CD8⁺ T-cells using 5 μg/ml of polybrene, with the CD8⁺ T-cells to be purified by magnetic separation (Miltenyi Biotec) from healthy donors or melanoma patients 24 h in advance and activated overnight with CD3/CD28 beads (Dynabeads, Life Technologies) at 3:1 bead: T-cell ratio. The following day, 900 μl of the media is removed and replaced with 500 μl of MC.27.759S TCR lentivirus supernatant in the presence of 5 μg/ml polybrene (Santa Cruz Biotechnology). Seven days later, T-cells that had taken up the virus are selected by enrichment with anti-rCD2 PE Ab (OX-34, BioLegend) followed by anti-PE magnetic beads (Miltenyi Biotec). 14 d post transduction T-cells are expanded with allogeneic feeders. For all functional experiments, MC.27.759S TCR transduced T-cells are >95% rCD2⁺ and used for functional analysis. Transduced cells are incubated with target cells for 4-5 h in the presence of 30 mM of TAPI-0 CD107a Ab and then stained with Abs for TNFα, CD3, CD8 and also Vivid. Gating is performed on size, single, vivid⁻CD3⁺ cells and then CD8⁺ versus CD107a or TNFα. Data acquisition and analysis is performed as above. The TCR transduced T-cells from patients are also used for chromium release cytotoxicity assays as described above.

### Example 8

### Design of a Mutant TCRs

A mutant version of the MC.27.759S TCR and its soluble form may be designed and prepared in which novel cysteine residues were introduced into the constant region within the extracellular region of each of the α and β chains. These mutations are made in order to form a novel disulfide bridge between the chains and hence promote pairing of the recombinant chains and reduce pairing with the endogenous TCR. See SEQ ID NOs: 11 and 13.

A further mutant version of the MC.27.759S TCR α chain may be designed and prepared in which, in addition to the introduction of a novel cysteine residue as described above, a cysteine residue is removed (by replacement with an alanine residue) and an asparagine residue is replaced by an aspartic acid residue. These further mutations are expected to provide advantages in terms of improved stability, particularly of the soluble form. See SEQ ID NO: 12.

### Example 9

### Preparation of an Artificial T-Cell Expressing the TCR of the Invention

Expression of the MC.27.759S in patients' T-cells is a potential therapeutic approach for cancer immunotherapy. DNA encoding the α and β chains of the T-cell receptor, separated with a cleavable read-through linker, such as those of the 2A family can be expressed in a lentiviral vector. An example DNA to be expressed has the sequence of SEQ ID NO: 16 and the corresponding encoded fusion protein has the sequence of SEQ ID NO: 30. This vector when transduced into patients' CD8 T-cells will drive expression of the MC.27.759S T-cell receptor (TCR) α and β chains at a 1:1 stoichiometry and thus allow a mature functional MC.27.759S TCR to be expressed on the cell surface. To produce CD8 T-cells expressing a MC.27.759S recombinant receptor the following procedures are followed. Firstly, the DNA sequences of the TCR are synthesised from assembled oligonucleotides and cloned into a lentiviral vector. Features of the lentiviral system to be utilised include a lentiviral vector incorporating self-inactivating 5′ and 3′ long terminal repeats (LTR). The viral vector encodes the entire coding sequence, including the signal sequences, of the TCR α and β chains separated by a read-through self-cleaving linker of the 2A family. The transcription of the TCR chains is driven by a constitutive promoter such as the cytomegalovirus (CMV) promoter or elongation factor 1a (EF1a) promoter.

The MC.27.759S encoding lentiviral plasmid vector is packaged into a virus using three additional packaging plasmids. The packaging plasmids encode 1) gag-pol 2) rev and 3) the envelope protein of vesicular stomatitis virus (VSV-G). Separation of the virus genome into 4 plasmids increases the safety of the virus and minimises the chances of recombination events leading to replication competent lentivirus (RCL) formation. Using cationic lipid transfection reagents, the vector and three packaging plasmids are simultaneously transfected into the packaging cell line HEK293T, in which replication-incompetent MC.27.759S TCR encoding lentivirus is assembled and secreted into the packaging cell culture supernatant. HEK293T cells may be grown attached to plastic or adapted for growth in suspension. Virus particles are concentrated from the cell supernatant by high speed centrifugation, and in some cases may be filtered through 0.45 μm pore filters to remove cellular debris and cryopreserved prior to transduction of CD8 T-cells.

To purify patient peripheral blood CD8 T-cells, apheresis is first performed on patients to isolate large numbers of peripheral blood mononuclear cells (PMBC) typically in the range of 10-20 billion mononuclear cells per patient. CD8 T-cells can isolated from these PBMCs using magnetic bead isolation. Microscopic paramagnetic beads coated with antibodies against CD8 are incubated with the PBMC, followed by separation on columns within a strong magnetic field. CD8-negative cells are eluted from the column by washing with serum albumin-containing phosphate buffered saline (SA/PBS). Following washing, the CD8 bead-labelled cells are released by removal from the magnet and washing with SA/PBS. The CD8 T-cells are then enumerated and incubated in cell culture medium in advance of activation and transduction with lentivirus encoding the TCR of the invention. Transduction of CD8 T-cells with TCR-encoding lentiviral particles is achieved by incubating CD8 T-cells in cell culture media into which lentiviral particles are added. CD8 cells may be in the resting state or pre-activated with antibodies against CD3 and CD28 with addition of cytokines including interleukin-2 and interleukin-7. Following 24-48 hours after transduction the cells are expanded for infusion back into patients. Expansion is performed with antibodies against CD3 and CD28, either in soluble or bead-bound form with addition of interleukin-2 for 14 days. In addition to interleukin-2, other cytokines such as interleukin-7 may also be added to the cultures. Expansion may also be performed using autologous feeder PBMCs which are irradiated with 30 Gray gamma irradiation to inhibit proliferation plus anti-CD3 antibody plus interleukin-2 at 3000-6000 IU/ml for 14 days.

### Example 10

### Applications of a Soluble Form of the TCR of the Invention

Solubilisation of TCRs opens up the ability to utilise TCRs in a number of applications, to include but not limited to, precise affinity measurements of the TCR for its ligand, crystallography-based structure solving, multimerization, and the generation of clinical therapies utilising the TCR as a targeting mechanism.

### Generation of Soluble, Monomeric TCR for Further Application

Expression of a soluble MC.27.759S TCR (“759S sTCR”), or a soluble mutant form as described in Example 8, could be used to characterise binding kinetics to its cognate or other MR1-bound ligands. Briefly, 759S sTCR can be tested by surface plasmon resonance (SPR), biolayer interferometry (BLI), or similar, to determine biophysical kinetics of the interaction between the soluble TCR and an MR1-ligand complex. For example, in the case of SPR, the ligand-MR1 complex is immobilised on specialised biosensor chips and concentrated 759S sTCR is passed over the chip and the interaction measured by SPR. From this, the affinity and binding half-life can be determined by calculation of the on-(kₐ or kₒₙ) and off-rates (k_{d} or K_{off}).

Generation of Multimeric sTCRs for Further Application

The DNA sequence of a 759S sTCR can also be modified for example to contain an acceptor tag peptide for biotin at the C-terminus of one of the chains. Following expression of such a construct, this acceptor tag can specifically be labelled with biotin via an enzymatic reaction using a biotin ligase such as BirA. Such a method will create a uniform biotinylation of the 759S sTCR which will allow it to be multimerised via binding to streptavidin, for generation of tetramers, pentamers or dextramers. These multimers can be further modified via the addition of a molecule such as phycoerythrin (PE), a fluorophore that allows detection of the multimer via fluorescence. The 759S sTCR multimeric complex could then be used to identify ligand-MR1 expressing cells via incubation of the molecule with target cells, and detection and/or sorting via methods such as fluorescent activated cell sorting (FACS).

Generation of Modified sTCRs for Potential Therapeutic Application

The 759S sTCR binding to its cognate ligand in vivo is unlikely to confer any therapeutic benefit. In order to generate a therapeutic agent with clinical effect, further modifications of the sTCR are required. The 759S sTCR recognises its target on a number of different cancer cells and cancer cell-derived cell lines. Therefore, to generate a reagent with therapeutic utility in the oncology setting, a 759S sTCR would need to have a function introduced that would induce cell death of the target cell, either directly or indirectly.

To modify 759S sTCR for this purpose, an effector function could be introduced, such as an anti-CD3 agonistic antibody, to engage and redirect T-cell activity. An anti-CD3 antibody optionally in scFv format could be added to the N or C terminus of either the α or β chain of the 759S sTCR, via a short, non-immunogenic linker sequence (such as GGGGS, SEQ ID NO: 17). Such a fusion protein would have bispecific properties.

### Example 11—Identification and Cloning of Further MR1-Reactive T-Cells A3, 4, C1, C2 and Demonstration of Response to a Wide Range of Tumour Targets and not to Normal Cells

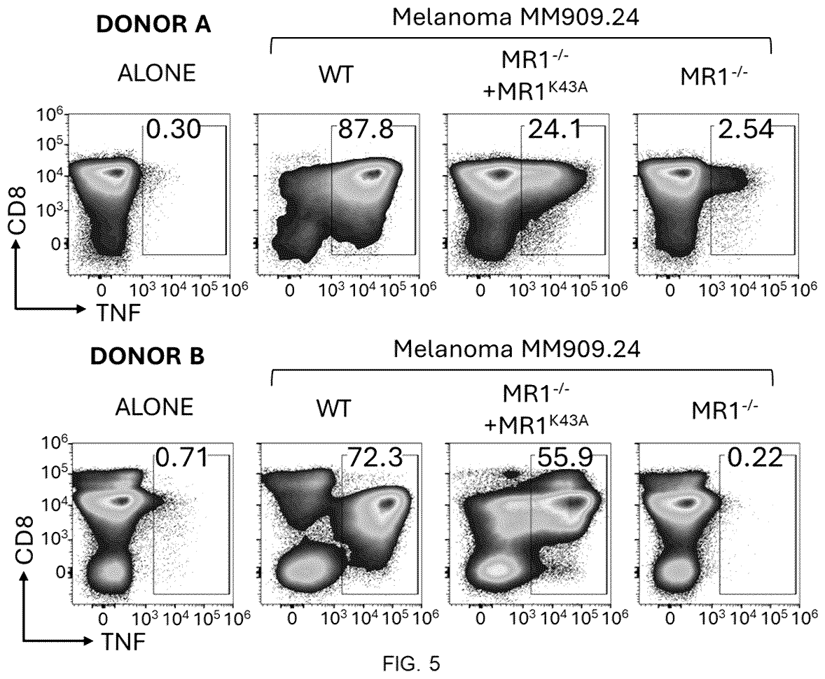

Further MR1-reactive T-cells (clones A3, 4, C1, C2) were identified in healthy donors, designated Donor A and Donor C herein, by priming of HLA-A2-negative PBMCs with C1R.A2 cells transduced with MR1 (C1R.A2.MR1) using the same methods as performed for the identification of clone MC.27.759S. Donor T cell populations were able to secrete TNFα cytokine and express CD69 in response to MM909.24 wildtype (FIG. 5) but were minimally stimulated when exposed to MM909.24 cells overexpressing MR1 with a K43A mutation or where MR1 was knocked out, therefore appearing to show ligand specificity in the context of MR1. T-cell clones, designated A3, A4 (from Donor A), C1, C2 (from Donor C), were isolated by flow cytometry sorting on TNFα+CD107a+ T-cells in response to C1R.MR1 cells from the respective donors as described and TCR α and β chain sequences were elucidated as describe for MC.27.759S. For the purposes of T-cell transduction and activity determination the 759S clone (V α and V β=SEQ ID NO: 9 and 10), A3 clone (V α and V β=SEQ ID NO: 53 and 54), A4 clone (V α and V β=SEQ ID NO: 55 and 56, C1 clone (V α and V β=SEQ ID NO: 57 and 58), and C2 clone (V α and V β=SEQ ID NO: 59 and 60), TCRs were synthesised with full length alpha and beta TCR chains comprising variable alpha region+constant murine alpha region and variable beta region+constant human beta region separated by a self-cleaving 2A sequence. The TCR constructs were cloned into a pSF-lenti tranfer plasmid backbone, which was modified to include an EF-1alpha promoter. The TCR vector cassette additionally contained a P2A and furin cleavable linker, a JIN transmembrane mutation to enhance hydrophobicity and constant domains murinised to improve TCR pairing and surface expression. CD3+ T-cells were magnetically isolated from healthy donor PBMCs and activated and expanded in the presence of CD3/CD28 Dynabeads. One day later, the cells were transduced with comparable virus particle number based on p24 measurement for the different vector constructs. TCR expression was confirmed 9 days later by staining with a VB-specific antibody and Flow-Cytometry analysis. Transduced cells comprising MC.7.G5 (see WO2019/097244; referred to as “MC.7.G5”) was also prepared in the same manner for use as an activity comparator and control.

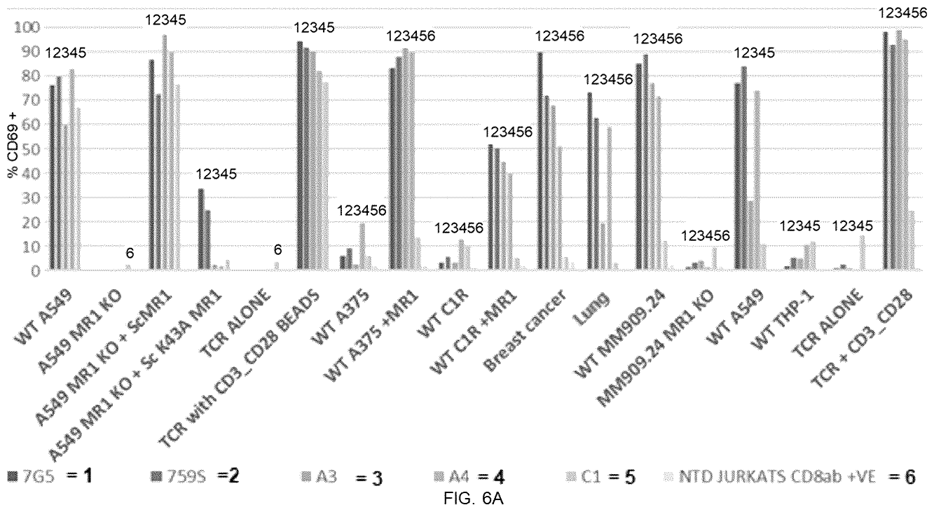

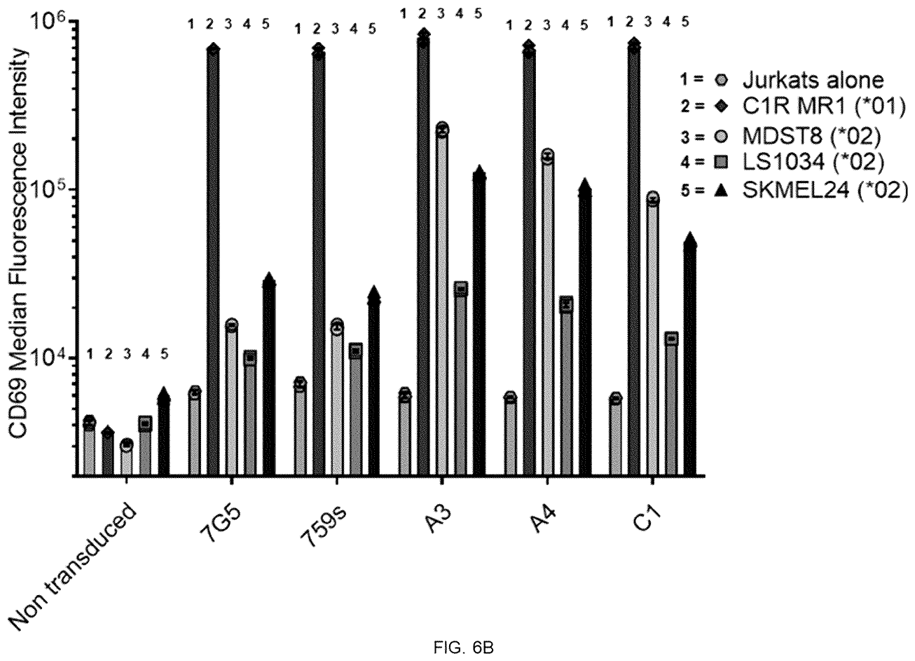

The TCR construct transduced T cells were expanded as previously described. 7G5, 759S, A3, A4 and C1 TCR+ and non-transduced T cells were then co-cultured with a variety of cancer origin cells, in some cases MR1-expressing or MR1 knock out cells as indicated in Table 2 below. CD69 expression was determined by flow cytometry. CD69 is an activation marker expressed by T cells early following exposure to an activating stimulus, CD69 expression is inducible within 3 hours and is expressed in the membrane at levels that allow easy detection by flow cytometry. T cells only and anti-CD3/CD28 stimulation were included as negative and positive controls (FIG. 6A and FIG. 6B).

Conclusions: Clear and specific CD69 response against a broad range of target cells was detected for each transduced construct, MC.27.759S, A3, A4, and C1, there was no detectible CD69 response above background (un-transduced T-cells), additionally the NTD (non-transduced) do not upregulate CD69 with the positive control CD3/CD28 as they don't have a TCR, K43A sensitivity was also apparent for the constructs. The data indicates that MC.27.759S, A3, A4, and C1 produce an activation-induced functional response to target a broad range of cancer cells with an effectiveness comparable to the cancer specific MR1 restricted MC.7.G5, no clone responds in absence of MR1 (knockout target cell data).

TABLE 2
Test cells and controls used for the activity assay of transduced T-cells
comprising the MR1 specific TCRs of clones MC.27.759S, A3, A4,
and C1 as illustrated in FIG. 6A and FIG. 6B and Example 11.
TEST CELL TYPE or CONTROL

WT A549 = MR1 expressing lung cancer cell
A549 MR1 KO = MR1 knockout
A549 MR1 KO + scMR1 = MR1 knockout with beta2 microglobulin MR1
fusion knock in
TCR alone = TCR transduced cells without any target cell, negative
control
TCR with CD3-CD28 beads = positive T cell activation control
WT A375 = Melanoma cell with low MR1 expression
A375 + MR1 = MR1 knock in
WT C1R = Lymphoblastoid cell with low MR1 expression, HLA*01
restricted
C1R + MR1 = MR1 knock in
Breast cancer = metastatic breast carcinoma cell
Lung = metastatic non-small cell lung cancer cell
WT MM909.24 = metastatic melanoma cell line which expresses MR1
MM909.24 MR1 KO = MR1 knockout
THP-1 = monocyte cancer cell
Jurkat = immortalized line of human T lymphocyte cells
MDST8 = colon cancer cell, HLA*02 restricted
LS1034 = colorectal cancer cell HLA*02 restricted
SKMEL24 = Melanoma cell HLA*02 restricted

It is understood that the methodologies of Example 5 to 10 applied in the context of clone MC.27.759S, of may equally be applied to the TCRs or fragments thereof derived from clones A2, A3, C1, C2.

### REFERENCES

Chua W J, Kim S, Myers N, Huang S, Yu L, Fremont D H, et al. (2011) Endogenous MHC-related protein 1 is transiently expressed on the plasma membrane in a conformation that activates mucosal-associated invariant T cells. Journal of Immunology, 186, 4744-50.
Crowther, M. D., Dolton, G., Legut, M., Caillaud, M. E., Lloyd, A., Attaf, M., Galloway, S. A., Rius, C., Farrell, C. P., Szomolay, B. and Ager, A., 2020. Genome-wide CRISPR-Cas9 screening reveals ubiquitous T cell cancer targeting via the monomorphic MHC class I-related protein MR1. Nature Immunology, 21 (2), pp. 178-185.
Ekeruche-Makinde, J., M. Clement, D. K. Cole, E. S. J. Edwards, K. Ladell, J. J. Miles, K. K. Matthews, A. Fuller, K. A. Lloyd, F. Madura, G. M. Dolton, J. Pentier, A. Lissina, E. Gostick, T. K. Baxter, B. M. Baker, P. J. Rizkallah, D. A Price, L. Wooldridge and A. K. Sewell (2012). T cell receptor optimized skewing of the repertoire can enhance antigen targeting. Journal of Biological Chemistry, 287, 37269-81.
Guo T, Chamoto K, Hirano N. (2015) Adoptive T Cell Therapy Targeting CD1 and MR1. Frontiers in immunology. 6:247.
Laugel B, Lloyd A, Meermeier E W, Crowther M D, Connor T R, Dolton G, et al. (2016) Engineering of Isogenic Cells Deficient for MR1 with a CRISPR/Cas9 Lentiviral System: Tools To Study Microbial Antigen Processing and Presentation to Human MR1-Restricted T Cells. Journal of Immunology, 197, 971-82.
Li W., H. Xu, T. Xiao, L. Cong, M. I. Love, F. Zhang, R. A. Irizarry, J. S. Liu, M. Brown, X. S. Liu, (2014) MAGeCK enables robust identification of essential genes from genome-scale CRISPR/Cas9 knockout screens. Genome Biology. 15, 554.
Lissina A., K. Ladell, A. Skowera, M. Clement, E. Edwards, R. Seggwiss, H. van den Berg, E. Gostick, K. Gallagher, E. Jones, J. J. Melenhorst, A. J. Godkin, M. Peakman, D. A. Price, A. K. Sewell and L. Wooldridge (2009) Protein kinase inhibitors substantially improve the physical detection of T-cells with peptide-MHC tetramers. Journal of Immunological Methods, 340, 11-24.
Parra-Cuadrado J F, Navarro P, Mirones I, Setien F, Oteo M, Martinez-Naves E. A study on the polymorphism of human MHC class I-related MR1 gene and identification of an MR1-like pseudogene. Tissue Antigens. 2000; 56 (2): 170-2.
Patel, S. J. et al 2012 Identification of essential genes for cancer immunotherapy. Nature, 584, 537-542.
R. Reantragoon, A. J. Corbett, I. G. Sakala, N. a Gherardin, J. B. Furness, Z. Chen, S. B. G. Eckle, A. P. Uldrich, R. W. Birkinshaw, O. Patel, L. Kostenko, B. Meehan, K. Kedzierska, L. Liu, D. P. Fairlie, T. H. Hansen, D. I. Godfrey, J. Rossjohn, J. Mccluskey, L. Kjer-Nielsen, Antigen-loaded MR1 tetramers define T cell receptor heterogeneity in mucosal-associated invariant T cells. The Journal of experimental medicine. 210, 2305-2320 (2013).
Ryan M. D., A. M. Q. King, G. P. Thomas, (1991) Cleavage of foot-and-mouth disease virus polyprotein is mediated by residues located within a 19 amino acid sequence. Journal of General Virology. 72, 2727-2732.
Sanjana N. E., O. Shalem, F. Zhang, (2014) Improved vectors and genome-wide libraries for CRISPR screening. Nature Methods. 11, 783-784.
Shalem, N. E. Sanjana, E. Hartenian, X. Shi, D. A. Scott, T. S. Mikkelsen, D. Heckl, B. L. Ebert, D. E. Root, J. G. Doench, F. Zhang, (2014) Genome-scale CRISPR-Cas9 knockout screening in human cells. Science. 343, 84-87.
Theaker, S. M., C. Rius, A. Greenshields-Watson, A. Lloyd, A. Trimby, A. Fuller, J. J. Miles, D. K. Cole, M. Peakman and A. K. Sewell G Dolton (2016) T-cell libraries allow simple parallel generation of multiple peptide-specific human T-cell clones. Journal of Immunological Methods 430, 43-50
Tungatt, K., V. Bianchi, M. D. Crowther, W. E. Powell, A. J. Schauenburg, A. Trimby, M. Donia, A. Skowera, J. J. Miles, C. J. Holland, D. K. Cole, A. J. Godkin, M. Peakman, P. T. Straten, I. M. Svane, A. K. Sewell and G, Dolton (2015) Antibody stabilization of peptide-MHC multimers reveals functional T-cells bearing extremely low affinity TCRs. Journal of Immunology 194, 463-74.
Wooldridge L., M. Clement, A. Lissina, E. S. J. Edwards, K. Ladell, J. Ekeruche, R. E. Hewitt, B. Laugel, E. Gostick, D. K. Cole, R. Debets, C. Berrevoets, J. J. Miles, S. R. Burrows, D. A. Price, A. K. Sewell (2010) MHC Class I Molecules with Superenhanced CD8 Binding Properties Bypass the Requirement for Cognate TCR Recognition and Nonspecifically Activate CTLs. The Journal of Immunology. 184, 3357-3366.
Wooldridge. L., J. Ekeruche-Makinde, H. A. van den Berg, A. Skowera, J. J. Miles, M. P. Tan, G. Dolton, M. Clement, S. Llewellyn-Lacey, D. A. Price, M. Peakman and A. K. Sewell (2012) A single autoimmune T-cell receptor recognises over a million different peptides. Journal of Biological Chemistry, 287, 1168-77.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

The invention embraces all combinations of preferred and more preferred groups and suitable and more suitable groups and embodiments of groups recited above.

1. A cancer-specific T-cell receptor (TCR) or a cancer-specific binding fragment of a TCR which binds a tumor antigen, wherein the TCR or binding fragment comprises:
(a) an alpha chain comprising a CDR3α comprising or consisting of CAVRLAGYGGSQGNLIF, (SEQ ID NO: 1) or a variant CDR3α that has 1, 2, 3, 4 or 5 amino acid variations with respect thereto; and/or a beta chain comprising a CDR3β comprising or consisting of CASSSQGTDTQYF, (SEQ ID NO:2) or a variant CDR3β that has 1, 2, 3, 4 or 5 amino acid variations with respect thereto,
(b) an alpha chain comprising a CDR3α comprising or consisting of CALSSYHYGGSQGNLIF (SEQ ID NO: 33) or a variant CDR3α that has 1, 2, 3, 4 or 5 variations amino acid variations with respect thereto; and/or a beta chain comprising a CDR3β comprising or consisting of CASRGNTGELFF (SEQ ID NO: 36) or a variant CDR3β that has 1, 2, 3, 4 or 5 amino acid variations with respect thereto, or
(c) an alpha chain comprising a CDR3α comprising or consisting of CALSSYHYGGSQGNLIF (SEQ ID NO: 33) or a variant CDR3α that has 1, 2, 3, 4 or 5 variations with respect thereto; and/or a beta chain comprising a CDR3β comprising or consisting of CASRTGQGNQPQHF (SEQ ID NO: 37) or a variant CDR3β that has 1, 2, 3, 4 or 5 amino acid variations with respect thereto,
(d) an alpha chain comprising a CDR3α comprising or consisting of CAVREADYGGSQGNLIF (SEQ ID NO: 34) or a variant CDR3α that has 1, 2, 3, 4 or 5 variations with respect thereto; and/or a beta chain comprising a CDR3β comprising or consisting of CASSLEQGQYF (SEQ ID NO: 38) or a variant CDR3β that has 1, 2, 3, 4 or 5 amino acid variations with respect thereto, or
(e) an alpha chain comprising a CDR3α comprising or consisting of CALSSYHYGGSQGNLIF (SEQ ID NO: 33) or a variant CDR3α that has 1, 2, 3, 4 or 5 amino acid variations with respect thereto, such as 1 or 2 variations, such as 1 variation; and/or a beta chain comprising a CDR3β comprising or consisting of CASSLEQGQYF (SEQ ID NO: 38) or a variant CDR3β that has 1, 2, 3, 4 or 5 amino acid variations with respect thereto, wherein in each case the variations are selected from additions, substitutions and deletions.

2. The TCR or cancer-specific binding fragment of a TCR according to claim 1, wherein the TCR or binding fragment comprises:
(a) one or more of the following CDRs comprising or consisting of; DSAIYN, (SEQ ID NO: 3), IQSSQREQ, (SEQ ID NO: 4), SGHDY, (SEQ ID NO: 5), and FNNNVP, (SEQ ID NO: 6), or a variant CDR that has 1 or 2 amino acid variations, such as 1 variation with respect thereto,
(b) one or more of the following CDRs comprising or consisting of; ATGYPS, (SEQ ID NO: 39), ATKADDK, (SEQ ID NO: 40), SGHDY, (SEQ ID NO: 5), and FNNNVP, (SEQ ID NO: 6), or a variant CDR that has 1 or 2 amino acid variations, such as 1 variation with respect thereto,
(c) one or more of the following CDRs comprising or consisting of; ATGYPS, (SEQ ID NO: 39), ATKADDK, (SEQ ID NO: 40), SGHDY, (SEQ ID NO: 5), and FNNNVP, (SEQ ID NO: 6), or a variant CDR that has 1 or 2 amino acid variations, such as 1 variation with respect thereto
(d) one or more of the following CDRs comprising or consisting of; VGISA, (SEQ ID NO: 41), LSSGK, (SEQ ID NO: 42), MDHEN, (SEQ ID NO: 43), and SYDVKM, (SEQ ID NO: 44), or a variant CDR that has 1 or 2 amino acid variations, such as 1 variation with respect thereto; or
(e) one or more of the following CDRs comprising or consisting of; ATGYPS, (SEQ ID NO: 39), ATKADDK, (SEQ ID NO: 40), MDHEN, (SEQ ID NO: 43), and SYDVKM, (SEQ ID NO: 44); or a variant CDR that has 1 or 2 amino acid variations, such as 1 variation with respect thereto; wherein in each case the variations are selected from additions, substitutions and deletions.

3. The TCR or cancer-specific binding fragment of a TCR according to claim 1, wherein the TCR or binding fragment comprises:
(a) 3 α-chain CDRs comprising or consisting of CAVRLAGYGGSQGNLIF, (SEQ ID NO: 1), or a variant CDR that has 1, 2, 3, 4 or 5 amino acid variations with respect thereto; DSAIYN, (SEQ ID NO: 3) and IQSSQREQ, (SEQ ID NO: 4) or a variant CDR that has 1 or 2 amino acid variations with respect thereto, such as 1 variation; and/or comprises 3 β-chain CDRs comprising or consisting of CASSSQGTDTQYF, (SEQ ID NO: 2) or a variant CDR that has 1, 2, 3, 4 or 5 amino acid variations with respect thereto, and SGHDY, (SEQ ID NO: 5) and FNNNVP, (SEQ ID NO: 6) or a variant CDR that has 1 or 2 amino acid variations with respect thereto, such as 1 variation,
(b) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and/or comprises 3 β-chain CDRs comprising or consisting of CASRGNTGELFF (SEQ ID NO: 36), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation;
(c) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and/or comprises 3 β-chain CDRs comprising or consisting of CASRTGQGNQPQHF (SEQ ID NO: 37), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation;
(d) 3 α-chain CDRs comprising or consisting of CAVREADYGGSQGNLIF (SEQ ID NO: 34) or a variant CDR that has 1, 2, 3, 4 or 5 amino acid variations with respect thereto; and VGISA, (SEQ ID NO: 41) and LSSGK, (SEQ ID NO: 42) or a variant CDR that has 1 or 2 amino acid variations with respect thereto, such as 1 variation; and/or comprises 3-β chain CDRs comprising or consisting of CASSLEQGQYF (SEQ ID NO: 38), or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; or
(e) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation and/or comprises 3-β chain CDRs comprising or consisting of CASSLEQGQYF (SEQ ID NO: 38) or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; wherein in each case the variations are selected from additions, substitutions and deletions.

4. The TCR or cancer-specific binding fragment of a TCR according to claim 1, wherein the TCR or binding fragment is not expressed by or associated with a mucosal-associated invariant T-cell (MAIT cell).

5. The TCR or cancer-specific binding fragment of a TCR according to claim 1, wherein the TCR or binding fragment comprises an α chain extracellular region comprising or consisting of:
(a)_
(SEQ ID NO: 9)
KQEVTQIPAALSVPEGENLVLNCSFTDSAIYNLQWFRQDPGKGLTSLLL
IQSSQREQTSGRLNASLDKSSGRSTLYIAASQPGDSATYLCAVRLAGYG
GSQGNLIFGKGTKLSVKPNIQNPDPAVYQLRDSKSSDKSVCLFTDFDSQ
TNVSQSKDSDVYITDKTVLDMRSMDFKSNSAVAWSNKSDFACANAFNNS
IIPEDTFFPSPESSCDVKLVEKSFETDTNLNFQNLSVIGFR.,
(b)_
(SEQ ID NO: 53)
DSVTQMEGPVTLSEEAFLTINCTYTATGYPSLFWYVQYPGEGLQLLLKA
TKADDKGSNKGFEATYRKETTSFHLEKGSVQVSDSAVYFCALSSYHYGG
SQGNLIFGKGTKLSVKPNIQNPDPAVYQLRDSKSSDKSVCLFTDFDSQT
NVSQSKDSDVYITDKTVLDMRSMDFKSNSAVAWSNKSDFACANAFNNSI
IPEDTFFPSPESSCDVKLVEKSFETDTNLNFQNLSVIGFR,
(c)_
(SEQ ID NO: 55)
DSVTQMEGPVTLSEEAFLTINCTYTATGYPSLFWYVQYPGEGLQLLLKA
TKADDKGSNKGFEATYRKETTSFHLEKGSVQVSDSAVYFCALSSYHYGG
SQGNLIFGKGTKLSVKPNIQNPDPAVYQLRDSKSSDKSVCLFTDFDSQT
NVSQSKDSDVYITDKTVLDMRSMDFKSNSAVAWSNKSDFACANAFNNSI
IPEDTFFPSPESSCDVKLVEKSFETDTNLNFQNLSVIGFR,
(d)_
(SEQ ID NO: 57)
NEVEQSPQNLTAQEGEFITINCSYSVGISALHWLQQHPGGGIVSLFMLS
SGKKKHGRLIATINIQEKHSSLHITASHPRDSAVYICAVRLAGYGGSQG
NLIFGKGTKLSVKPNIQNPDPAVYQLRDSKSSDKSVCLFTDFDSQTNVS
QSKDSDVYITDKTVLDMRSMDFKSNSAVAWSNKSDFACANAFNNSIIPE
DTFFPSPESSCDVKLVEKSFETDTNLNFQNLSVIGFR,;
or
(e)_
(SEQ ID NO: 59)
DSVTQMEGPVTLSEEAFLTINCTYTATGYPSLFWYVQYPGEGLQLLLKA
TKADDKGSNKGFEATYRKETTSFHLEKGSVQVSDSAVYFCALSSYHYGG
SQGNLIFGKGTKLSVKPNIQNPDPAVYQLRDSKSSDKSVCLFTDFDSQT
NVSQSKDSDVYITDKTVLDMRSMDFKSNSAVAWSNKSDFACANAFNNSI
IPEDTFFPSPESSCDVKLVEKSFETDTNLNFQNLSVIGFR,;
or a variant α chain extracellular region which has at least 85% sequence identity to the α chain extracellular region of SEQ ID NO: 9, 53, 55, 57 or 59.

6. The TCR or cancer-specific binding fragment of a TCR according to claim 1, wherein the TCR comprises a β chain extracellular region comprising or consisting of:
(a)_
(SEQ ID NO: 10)
DAGVIQSPRHEVTEMGQEVTLRCKPISGHDYLFWYRQTMMRGLELLIYF
NNNVPIDDSGMPEDRFSAKMPNASFSTLKIQPSEPRDSAVYFCASSSQG
TDTQYFGPGTRLTVLEDLKNVFPPKVAVFEPSEAEISHTQKATLVCLAT
GFYPDHVELSWWVNGKEVHSGVSTDPQPLKEQPALNDSRYCLSSRLRVS
ATFWQNPRNHFRCQVQFYGLSENDEWTQDRAKPVTQIVSAEAWGRADCG
FTSESYQQGVLSATILYE.,
(b)_
(SEQ ID NO: 54)
GVIQSPRHEVTEMGQEVTLRCKPISGHDYLFWYRQTMMRGLELLIYFNN
NVPIDDSGMPEDRFSAKMPNASFSTLKIQPSEPRDSAVYFCASSLCASR
GNTGELFFGEGSRLTVLEDLKNVFPPKVAVFEPSEAEISHTQKATLVCL
ATGFYPDHVELSWWVNGKEVHSGVSTDPQPLKEQPALNDSRYCLSSRLR
VSATFWQNPRNHFRCQVQFYGLSENDEWTQDRAKPVTQIVSAEAWGRAD
CGFTSESYQQGVLSATILYE,,
(c)_
(SEQ ID NO: 56)
GVIQSPRHEVTEMGQEVTLRCKPISGHDYLFWYRQTMMRGLELLIYFNN
NVPIDDSGMPEDRFSAKMPNASFSTLKIQPSEPRDSAVYFCASSLCASR
TGQGNQPQHFGDGTRLSILEDLKNVFPPKVAVFEPSEAEISHTQKATLV
CLATGFYPDHVELSWWVNGKEVHSGVSTDPQPLKEQPALNDSRYCLSSR
LRVSATFWQNPRNHFRCQVQFYGLSENDEWTQDRAKPVTQIVSAEAWGR
ADCGFTSESYQQGVLSATILYE,,
(d)_
(SEQ ID NO: 58)
SRYLVKRTGEKVFLECVQDMDHENMFWYRQDPGLGLRLIYFSYDVKMKE
KGDIPEGYSVSREKKERFSLILESASTNQTSMYLCASSLCASSLEQGQY
FGPGTRLLVLEDLKNVFPPKVAVFEPSEAEISHTQKATLVCLATGFYPD
HVELSWWVNGKEVHSGVSTDPQPLKEQPALNDSRYCLSSRLRVSATFWQ
NPRNHFRCQVQFYGLSENDEWTQDRAKPVTQIVSAEAWGRADCGFTSES
YQQGVLSATILYE,;
or
(e)_
(SEQ ID NO: 60)
SRYLVKRTGEKVFLECVQDMDHENMFWYRQDPGLGLRLIYFSYDVKMKE
KGDIPEGYSVSREKKERFSLILESASTNQTSMYLCASSLEQGQYFGPGT
RLLVLEDLKNVFPPKVAVFEPSEAEISHTQKATLVCLATGFYPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADCGFTSESYQQGV
LSATILYE,;
or a variant β chain extracellular region which has at least 85% sequence identity to the β chain extracellular region of SEQ ID NO: 10, 54, 56, 58 or 60.

7. The TCR or cancer-specific binding fragment of a TCR according to claim 1, wherein the TCR or binding fragment comprises:
(a) 3 α-chain CDRs comprising or consisting of CAVRLAGYGGSQGNLIF, (SEQ ID NO: 1) or a variant CDR that has 1, 2, 3, 4 or 5 amino acid variations with respect thereto; DSAIYN, (SEQ ID NO: 3) and IQSSQREQ, (SEQ ID NO: 4) or a variant CDR that has 1 or 2 amino acid variations with respect thereto, such as 1 variation; and comprises 3 β-chain CDRs comprising or consisting of CASSSQGTDTQYF, (SEQ ID NO: 2) or a variant CDR that has 1, 2, 3, 4 or 5 amino acid variations with respect thereto, and SGHDY, (SEQ ID NO: 5) and FNNNVP, (SEQ ID NO: 6) or a variant CDR that has 1 or 2 amino acid variations with respect thereto, such as 1 variation, or
(b) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRGNTGELFF (SEQ ID NO: 36), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation; or
(c) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation, and comprises 3 β-chain CDRs comprising or consisting of CASRTGQGNQPQHF (SEQ ID NO: 37), or a variant CDR that has 1, 2, 3, 4 or 5 amino acid variations with respect thereto, and SGHDY (SEQ ID NO: 5) and FNNNVP, (SEQ ID NO: 6) or a variant CDR that has 1 or 2 amino acid variations with respect thereto, such as 1 variation; or
(d) 3 α-chain CDRs comprising or consisting of CAVREADYGGSQGNLIF (SEQ ID NO: 34) or a variant CDR that has 1, 2, 3, 4 or 5 amino acid variations with respect thereto; and VGISA, (SEQ ID NO: 41) and LSSGK, (SEQ ID NO: 42) or a variant CDR that has 1 or 2 amino acid variations with respect thereto, such as 1 variation; and comprises 3-β chain CDRs comprising or consisting of CASSLEQGQYF (SEQ ID NO: 38), or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; or
(e) 3 α-chain CDRs comprising or consisting of CALSSYHYGGSQGNLIF (SEQ ID NO: 33) or a variant CDR that has 1, 2, 3, 4 or 5 amino acid variations with respect thereto; and ATGYPS, (SEQ ID NO: 39) and ATKADDK, (SEQ ID NO: 40), or a variant CDR that has 1 or 2 amino acid variations with respect thereto, such as 1 variation and comprises 3-β chain CDRs comprising or consisting of CASSLEQGQYF (SEQ ID NO: 38) or a variant CDR that has 1, 2, 3, 4 or 5 amino acid variations with respect thereto, and MDHEN (SEQ ID NO: 43) and SYDVKM, (SEQ ID NO: 44) or a variant CDR that has 1 or 2 amino acid variations with respect thereto, such as 1 variation; or
(f) an α chain extracellular region comprising or consisting of SEQ ID NO: 11 or a variant α chain extracellular region which has at least 85% sequence identity to the α chain extracellular region of SEQ ID NO: 11 and a β chain extracellular region comprising SEQ ID NO: 12 or a variant β chain extracellular region which has at least 85% sequence identity to the β chain extracellular region of SEQ ID NO: 12 or a β chain extracellular region comprising SEQ ID NO: 13 or a variant β chain extracellular region which has at least 85% sequence identity to the β chain extracellular region of SEQ ID NO: 13; or
(g) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising or consisting of SEQ ID NO: 10 or a variant β chain extracellular region which has at least 85% sequence identity to the β chain extracellular region of SEQ ID NO: 10; or
(h) an α chain comprising or consisting of SEQ ID NO: 29 or a variant α chain which has at least 85% sequence identity to the α chain of SEQ ID NO: 29 and a β chain comprising or consisting of SEQ ID NO: 31 or a variant β chain which has at least 85% sequence identity to the β chain of SEQ ID NO: 31; or
(i) an α chain extracellular region comprising or consisting of SEQ ID NO: 9 or a variant α chain extracellular region which has at least 85% sequence identity to the α chain extracellular region of SEQ ID NO: 9 and a β chain extracellular region comprising SEQ ID NO: 12 or a variant β chain extracellular region which has at least 85% sequence identity to the β chain extracellular region of SEQ ID NO: 12 or a β chain extracellular region comprising SEQ ID NO: 13 or a variant β chain extracellular region which has at least 85% sequence identity to the β chain extracellular region of SEQ ID NO: 13; or
(j) an α chain variable region comprising or consisting of SEQ ID NO: 14 or a variant α chain variable region which has at least 85% sequence identity to the α chain variable region of SEQ ID NO: 14 and a β chain variable region comprising or consisting of SEQ ID NO: 15 or a variant β chain variable region which has at least 85% sequence identity to the β chain variable region of SEQ ID NO: 15; or
(k) an α chain variable region comprising or consisting of SEQ ID NO: 45 or a variant α chain variable region which has at least 85% sequence identity to the α chain variable region of SEQ ID NO: 45 and a β chain variable region comprising or consisting of SEQ ID NO: 46 or a variant β chain variable region which has at least 85% sequence identity to the β chain variable region of SEQ ID NO: 46; or
(l) an α chain variable region comprising or consisting of SEQ ID NO: 47 or a variant α chain variable region which has at least 85% sequence identity to the α chain variable region of SEQ ID NO: 47 and a β chain variable region comprising or consisting of SEQ ID NO: 48 or a variant β chain variable region which has at least 85% sequence identity to the β chain variable region of SEQ ID NO: 48; or
(n) an α chain variable region comprising or consisting of SEQ ID NO: 49 or a variant α chain variable region which has at least 85% sequence identity to the α chain variable region of SEQ ID NO: 49 and a β chain variable region comprising or consisting of SEQ ID NO: 50 or a variant β chain variable region which has at least 85% sequence identity to the β chain variable region of SEQ ID NO: 50; or
(m) an α chain variable region comprising or consisting of SEQ ID NO: 51 or a variant α chain variable region which has at least 85% sequence identity to the α chain variable region of SEQ ID NO: 51 and a β chain variable region comprising or consisting of SEQ ID NO: 52 or a variant β chain variable region which has at least 85% sequence identity to the β chain variable region of SEQ ID NO: 52;
optionally wherein the variations may be selected from amino acid additions, substitutions and deletions.

8. The TCR or cancer-specific binding fragment of a TCR according to claim 1, wherein the amino acid sequence of the TCR or binding fragment is artificial.

9. The TCR or cancer-specific binding fragment of a TCR according to claim 1, wherein the TCR or binding fragment is a soluble form of a TCR or binding fragment.

10. The TCR or cancer-specific binding fragment of a TCR according to claim 1 wherein the TCR or cancer-specific binding fragment is MR1-restricted.

11. A polynucleotide encoding the TCR or cancer-specific binding fragment of a TCR according to claim 1.

12. (canceled)

13. A vector for delivery of a polynucleotide to cells comprising a polynucleotide according to claim 11, optionally wherein the vector is a viral vector.

14. An immune cell, such as a T-cell, particularly an engineered immune cell such as an engineered T-cell, expressing the TCR or cancer-specific binding fragment according to claim 1.

15. An immune cell clone, such as a T-cell clone, particularly an engineered immune cell clone such as an engineered T-cell clone, expressing the TCR or cancer-specific binding fragment of a TCR according to claim 1, optionally wherein the clone is a MC.27.759S, A3, A4, C1, or C2 clone.

16. (canceled)

17. An ex vivo process comprising (i) obtaining immune cells, particularly T-cells from a patient, (ii) optionally expanding the immune cells, particularly T-cells (iii) introducing a vector according to claim 13 into the immune cells, particularly T-cells to produce modified immune cells; and (iv) reintroducing the modified immune cells, particularly T-cells into the patient.

18. (canceled)

19. A pharmaceutical composition comprising the immune cell, particularly the T-cell according to claim 14 and a pharmaceutically acceptable carrier.

20. A bispecific construct comprising the TCR or cancer-specific binding fragment of a TCR according to claim 1 and an immune cell activating component or ligand that binds to and activates an immune cell.

21. A fusion protein comprising the TCR or cancer-specific binding fragment of a TCR according to claim 1 and a heterologous protein.

22. A method of treating cancer in a subject comprising administering a therapeutically effective amount of the immune cell, or T-cell according to claim 14, to the subject.

23. (canceled)

24. A pharmaceutical composition comprising:
a) the immune cell, or T-cell according to claim 14; and
b) an anti-cancer agent.
